SEQUENCE LISTING (38 sequences)

SEQ ID NO: 1 - RNA, 87 nt, Artificial
  mutant tRNA
gcccggaugguggaaucgguagacacaagggauucuaaaucccucggcguucgcgcugugcggguucaagucccgcuccggguacca

SEQ ID NO: 2 - DNA, 77 bp, Artificial
  mutant tRNA, T at position 61
ccggcgguaguucagcagggcagaacggcggacucuaaauccgcaaggcgcugguucaaatccggcucgccggacca

SEQ ID NO: 3 - protein, 860 aa, Escherichia coli [sequence omitted]

SEQ ID NO: 4 - protein, 306 aa, Methanococcus jannaschii
MDEFEMIKRNTSEIISEEELREVLKKDEKSAYIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNDIHYLGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 5 - protein, 860 aa, Artificial
  mutant tRNA synthetase
MQEQYRPEEIESKVQLHWDEKRTFEVTEDESKEKYYCLSAAPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWPYARYTCPQYKEGMLDSEAANYWLPVDIVIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVG

SEQ ID NO: 6 - protein, 860 aa, Artificial
  mutant tRNA synthetase
MQEQYRPEEIESKVQLHWDEKRTFEVTEDESKEKYYCLSVMPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWLYARYTCPQYKEGMLDSEAANYWLPVDILIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVG

SEQ ID NO: 7 - protein, 860 aa, Artificial
  mutant tRNA synthetase
MQEQYRPEEIESKVQLHWDEKRTFEVTEDESKEKYYCLSHPPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWAYARYTCPQYKEGMLDSEAANYWLPVDIMIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVG

SEQ ID NO: 8 - protein, 860 aa, Artificial
  mutant tRNA synthetase
MQEQYRPEEIESKVQLHWDEKRTFEVTEDESKEKYYCLSVYPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWLYARYTCPQYKEGMLDSEAANYWLPVDILIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVG

SEQ ID NO: 9 - protein, 860 aa, Artificial
  mutant tRNA synthetase
MQEQYRPEEIESKVQLHWDEKRTFEVTEDESKEKYYCLSLEPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWRYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVG

SEQ ID NO: 10 - protein, 860 aa, Artificial
  mutant tRNA synthetase
MQEQYRPEEIESKVQLHWDEKRTFEVTEDESKEKYYCLSMEPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWRYARYTCPQYKEGMLDSEAANYWLPVDIFIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVG

SEQ ID NO: 11 - protein, 860 aa, Artificial
  mutant tRNA synthetase
MQEQYRPEEIESKVQLHWDEKRTFEVTEDESKEKYYCLSLEPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWRYARYTCPQYKEGMLDSEAANYWLPVDICIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVG

SEQ ID NO: 12 - protein, 860 aa, Artificial
  mutant tRNA synthetase
MQEQYRPEEIESKVQLHWDEKRTFEVTEDESKEKYYCLSFEPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWRYARYTCPQYKEGMLDSEAANYWLPVDITIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVG

SEQ ID NO: 13 - protein, 860 aa, Artificial
  mutant tRNA synthetase
MQEQYRPEEIESKVQLHWDEKRTFEVTEDESKEKYYCLSGEPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWRYARYTCPQYKEGMLDSEAANYWLPVDILIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVG

SEQ ID NO: 14 - protein, 860 aa, Artificial
  mutant tRNA synthetase
MQEQYRPEEIESKVQLHWDEKRTFEVTEDESKEKYYCLSGWPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWAYARYTCPQYKEGMLDSEAANYWLPVDILIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVG

SEQ ID NO: 15 - protein, 860 aa, Artificial
  mutant tRNA synthetase
MQEQYRPEEIESKVQLHWDEKRTFEVTEDESKEKYYCLSWSPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVG

SEQ ID NO: 16 - protein, 860 aa, Artificial
  mutant tRNA synthetase
MQEQYRPEEIESKVQLHWDEKRTFEVTEDESKEKYYCLSGTPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWWYARYTCPQYKEGMLDSEAANYWLPVDILIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVG

SEQ ID NO: 17 - protein, 306 aa, Artificial
  mutant tRNA synthetase
MDEFEMIKRNTSEIISEEELREVLKKDEKSAGIGFEPSGKIHLGHYLQIKKMIDLQNAGFDIIIELADLHAYLNQKGELDEIRKIGDYNKKVFEAMGLKAKYVYGSEAELDKDYTLNVYRLALKTTLKRARRSMELIAREDENPKVAEVIYPIMQVNGIHYHGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 18 - DNA, 2583 bp, Escherichia coli
atgcaagagcaataccgcccggaagagatagaatccaaagtacagcttcattgggatgagaagcgcacatttgaagtaaccgaagacgagagcaaagagaagtattactgcctgtctatgcttccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggtactatgcgcgctacacttgcccgcagtacaaagaaggtatgctggattccgaagcggctaactactggctgccggtggatatctacattggtggtattgaacacgccattatgcacctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 19 - DNA, 921 bp, Methanococcus jannaschii
atggacgaatttgaaatgataaagagaaacacatctgaaattatcagcgaggaagagttaagagaggttttaaaaaaagatgaaaaatctgcttacataggttttgaaccaagtggtaaaatacatttagggcattatctccaaataaaaaagatgattgatttacaaaatgctggatttgatataattatattgttggctgatttacacgcctatttaaaccagaaaggagagttggatgagattagaaaaataggagattataacaaaaaagtttttgaagcaatggggttaaaggcaaaatatgtttatggaagtgaattccagcttgataaggattatacactgaatgtctatagattggctttaaaaactaccttaaaaagagcaagaaggagtatggaacttatagcaagagaggatgaaaatccaaaggttgctgaagttatctatccaataatgcaggttaatgatattcattatttaggcgttgatgttgcagttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttgaatatcctttaaccataaaaaggccagaaaaatttggtggagatttgacagttaatagctatgaggagttagagagtttatttaaaaataaggaattgcatccaatggatttaaaaaatgctgtagctgaagaacttataaagattttagagccaattagaaagagattataa

SEQ ID NO: 20 - DNA, 2583 bp, Artificial
  mutant tRNA synthetase
atgcaagagcaataccgcccggaagagatagaatccaaagtacagcttcattgggatgagaagcgcacatttgaagtaaccgaagacgagagcaaagagaagtattactgcctgtctgctgcgccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggccttatgcgcgctacacttgcccgcagtacaaagaaggtatgctggattccgaagcggctaactactggctgccggtggatatcgttattggtggtattgaacacgccattatggggctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 21 - DNA, 2583 bp, Artificial
  mutant tRNA synthetase
atgcaagagcaataccgcccggaagagatagaatccaaagtacagcttcattgggatgagaagcgcacatttgaagtaaccgaagacgagagcaaagagaagtattactgcctgtctgtgatgccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggctgtatgcgcgctacacttgcccgcagtacaaagaaggtatgctggattccgaagcggctaactactggctgccggtggatatcctgattggtggtattgaacacgccattatggggctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 22 - DNA, 2583 bp, Artificial
  mutant tRNA synthetase
atgcaagagcaataccgcccggaagagatagaatccaaagtacagcttcattgggatgagaagcgcacatttgaagtaaccgaagacgagagcaaagagaagtattactgcctgtctcatcctccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctgggcgtatgcgcgctacacttgcccgcagtacaaagaaggtatgctggattccgaagcggctaactactggctgccggtggatatcatgattggtggtattgaacacgccattatgggtctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 23 - DNA, 2583 bp, Artificial
  mutant tRNA synthetase
atgcaagagcaataccgcccggaagagatagaatccaaagtacagcttcattgggatgagaagcgcacatttgaagtaaccgaagacgagagcaaagagaagtattactgcctgtctgtgtatccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggctgtatgcgcgctacacttgcccgcagtacaaagaaggtatgctggattccgaagcggctaactactggctgccggtggatatcctgattggtggtattgaacacgccattatgggtctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 24 - DNA, 2583 bp, Artificial
  mutant tRNA synthetase
atgcaagagcaataccgcccggaagagatagaatccaaagtacagcttcattgggatgagaagcgcacatttgaagtaaccgaagacgagagcaaagagaagtattactgcctgtctttggagccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggcgttatgcgcgctacacttgcccgcagtacaaagaaggtatgctggattccgaagcggctaactactggctgccggtggatatcgctattggtggtattgaacacgccattatgggtctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 25 - DNA, 2583 bp, Artificial
  mutant tRNA synthetase
atgcaagagcaataccgcccggaagagatagaatccaaagtacagcttcattgggatgagaagcgcacatttgaagtaaccgaagacgagagcaaagagaagtattactgcctgtctatggagccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggcgttatgcgcgctacacttgcccgcagtacaaagaaggtatgctggattccgaagcggctaactactggctgccggtggatatctttattggtggtattgaacacgccattatggggctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 26 - DNA, 2583 bp, Artificial
  mutant tRNA synthetase
atgcaagagcaataccgcccggaagagatagaatccaaagtacagcttcattgggatgagaagcgcacatttgaagtaaccgaagacgagagcaaagagaagtattactgcctgtctttggagccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggcgttatgcgcgctacacttgcccgcagtacaaagaaggtatgctggattccgaagcggctaactactggctgccggtggatatctgtattggtggtattgaacacgccattatgggtctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 27 - DNA, 2583 bp, Artificial
  mutant tRNA synthetase
atgcaagagcaataccgcccggaagagatagaatccaaagtacagcttcattgggatgagaagcgcacatttgaagtaaccgaagacgagagcaaagagaagtattactgcctgtcttttgagccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggcgttatgcgcgctacacttgcccgcagtacaaagaaggtatgctggattccgaagcggctaactactggctgccggtggatatcacgattggtggtattgaacacgccattatgggtctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 28 - DNA, 2583 bp, Artificial
  mutant tRNA synthetase
atgcaagagcaataccgcccggaagagatagaatccaaagtacagcttcattgggatgagaagcgcacatttgaagtaaccgaagacgagagcaaagagaagtattactgcctgtctggggagccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggcggtatgcgcgctacacttgcccgcagtacaaagaaggtatgctggattccgaagcggctaactactggctgccggtggatatcctgattggtggtattgaacacgccattatgggtctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 29 - DNA, 2583 bp, Artificial
  mutant tRNA synthetase
atgcaagagcaataccgcccggaagagatagaatccaaagtacagcttcattgggatgagaagcgcacatttgaagtaaccgaagacgagagcaaagagaagtattactgcctgtctggttggccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctgggcttatgcgcgctacacttgcccgcagtacaaagaaggtatgctggattccgaagcggctaactactggctgccggtggatatccttattggtggtattgaacacgccattatgggtctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 30 - DNA, 2583 bp, Artificial
  mutant tRNA synthetase
atgcaagagcaataccgcccggaagagatagaatccaaagtacagcttcattgggatgagaagcgcacatttgaagtaaccgaagacgagagcaaagagaagtattactgcctgtcttggtcgccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggatttatgcgcgctacacttgcccgcagtacaaagaaggtatgctggattccgaagcggctaactactggctgccggtggatatcgcgattggtggtattgaacacgccattatggggctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 31 - DNA, 2583 bp, Artificial
  mutant tRNA synthetase
atgcaagagcaataccgcccggaagagatagaatccaaagtacagcttcattgggatgagaagcgcacatttgaagtaaccgaagacgagagcaaagagaagtattactgcctgtctggtacgccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggtggtatgcgcgctacacttgcccgcagtacaaagaaggtatgctggattccgaagcggctaactactggctgccggtggatatccttattggtggtattgaacacgccattatgggtctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 32 - DNA, 921 bp, Artificial
  mutant tRNA synthetase
atggacgaatttgaaatgataaagagaaacacatctgaaattatcagcgaggaagagttaagagaggttttaaaaaaagatgaaaaatctgctggtataggttttgaaccaagtggtaaaatacatttagggcattatctccaaataaaaaagatgattgatttacaaaatgctggatttgatataattatagagttggctgatttacacgcctatttaaaccagaaaggagagttggatgagattagaaaaataggagattataacaaaaaagtttttgaagcaatggggttaaaggcaaaatatgtttatggaagtgaagcggagcttgataaggattatacactgaatgtctatagattggctttaaaaactaccttaaaaagagcaagaaggagtatggaacttatagcaagagaggatgaaaatccaaaggttgctgaagttatctatccaataatgcaggttaatggtattcattatcatggcgttgatgttgcagttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttgaatatcctttaaccataaaaaggccagaaaaatttggtggagatttgacagttaatagctatgaggagttagagagtttatttaaaaataaggaattgcatccaatggatttaaaaaatgctgtagctgaagaacttataaagattttagagccaattagaaagagattataa

SEQ ID NO: 33 - DNA, 58 bp, Artificial
  oligonucleotide primer
gatcaccggtaagcttcccgataagggagcaggccagtaaaaagcattaccccgtgcc

SEQ ID NO: 34 - DNA, 54 bp, Artificial
  oligonucleotide primer
ggatttagaatcccttgtgtctaccgattccaccatccgggcacggggtaatgc

SEQ ID NO: 35 - DNA, 54 bp, Artificial
  oligonucleotide primer
agggattctaaatccctcggcgttcgcgctgtgcgggttcaagtcccgctccgg

SEQ ID NO: 36 - DNA, 58 bp, Artificial
  oligonucleotide primer
ttaggctagcgggaagttcagggacttttgaaaaaaatggtacccggagcgggacttg

SEQ ID NO: 37 - DNA, 40 bp, Artificial
  oligonucleotide primer
gcgcgaattcagtatggaagagcaataccgcccggaagag

SEQ ID NO: 38 - DNA, 36 bp, Artificial
  oligonucleotide primer
gcgcgcggccgcttagccaacgaccagattgaggag

(19) United States
(12) Patent Application Publication
(10) Pub. No.: US 2009/0181858 A1
(43) Pub. Date: Jul. 16, 2009

(54) Adding photoregulated amino acids to the genetic code

(75) Inventors: Alexander Deiters, Raleigh, NC (US); Ning Wu, Boston, MA (US); Peter G. Schultz, La Jolla, CA (US); David King, San Francisco, CA (US); T. Ashton Cropp, Bethesda, MD (US); Mohua Bose, Cupertino, CA (US); Dan Groff, San Diego, CA (US); Jianming Xie, Mountain View, CA (US); Eric Brustad, San Diego, CA (US)

Correspondence Address: QUINE INTELLECTUAL PROPERTY LAW GROUP, P.C., P O BOX 458, ALAMEDA, CA, 94501, US

(73) Assignee: The Scripps Research Institute, La Jolla, CA (US)

(21) Appl. No.: 11/662,749

(22) PCT Filed: Sep. 21, 2005
(86) PCT No.: PCT/US05/34002

§ 371 Date: Oct. 28, 2008

Related U.S. Application Data
(60) Provisional application No. 60/612,223, filed on Sep. 21, 2004.
(60) Provisional application No. 60/620,625, filed on Oct. 19, 2004.

(51) Int. Cl. C40B 30/06; C12N 9/00; C07K 2/00; C07H 21/00; C12Q 1/68; C12P 21/00; C40B 40/06; C40B 40/02; C40B 30/00

(52) U.S. Cl. 506/10; 435/183; 530/300; 536/23.2; 435/6; 435/69.1; 506/16; 506/14; 506/7

(57) ABSTRACT

Compositions and methods of producing components of protein biosynthetic machinery that include orthogonal leucyl-tRNAs, orthogonal leucyl-aminoacyl-tRNA synthetases, and orthogonal pairs of leucyl-tRNAs/synthetases, which incorporate photoregulated amino acids, OMe-L-tyrosine, α-aminocaprylic acid, or o-nitrobenzyl cysteine into proteins are proteins are provided in response to an amber selector codon. Methods for identifying these orthogonal pairs are also provided along with methods of producing proteins with a photoregulated amino acid, Ome-L-tyrosine, α-aminocaprylic acid, or o-nitrobenzyl cysteine using these orthogonal pairs.

### CROSS-REFERENCE TO RELATED APPLICATIONS

This application is related to and claims priority to and benefit of, Provisional Patent Applications U.S. Ser. No. 60/612,223, filed Sep. 21, 2004, and USSN 60-620,625, filed Oct. 19, 2004, the disclosures of which are incorporated herein by reference in their entirety for all purposes.

### STATEMENT AS TO RIGHTS TO INVENTIONS MADE UNDER FEDERALLY SPONSORED RESEARCH AND DEVELOPMENT

This invention was made with government support under Grant GM62159 from the National Institutes of Health and Grant ER46051 from the Department of Energy. The government may have certain rights to this invention.

### FIELD OF THE INVENTION

The invention is in the field of translation biochemistry. The invention relates to libraries, compositions and methods for producing and using orthogonal tRNAs, orthogonal aminoacyl-tRNA synthetases, and pairs thereof, that selectively incorporate unnatural amino acids, e.g., photoregulated amino acids, into proteins in response to selector codons such as stop selector codons or four base codons. This includes the incorporation of multiple different unnatural amino acids into a single protein chain in response to such codons. The invention also relates to methods of producing proteins in cells using such pairs and related compositions.

### BACKGROUND OF THE INVENTION

With few exceptions, the genetic codes of all known organisms encode the same twenty amino acids. This feature limits the use of naturally occurring amino acids in the development of novel chemistries for photoregulatable protein modification. To add a new amino acid to the repertoire of an organism requires a unique tRNA/aminoacyl-tRNA synthetase pair, a source of the amino acid, and a unique selector codon that specifies the amino acid (Furter (1998) Protein Sci., 7:419-426). Previously, the inventors and coworkers have shown that the amber nonsense codon, TAG, together with orthogonal M. jannaschii and E. coli tRNA/synthetase pairs can be used to genetically encode a variety of amino acids with novel properties in E. coli. Such approaches have proven feasible to add unnatural amino acids to proteins in the in vivo protein biosynthetic machinery of the eubacteria Escherichia coli (E. coli) and other organisms. See, e.g., Wang et al., 2000, J. Am. Chem. Soc., 122:5010-5011; Chin et al., 2002, J. Am. Chem. Soc, 124:9026-9027; Wang et al., 2001, Science, 292:498-500; Wang et al., 2003, Proc. Natl. Acad. Sci. USA, 100:56-61; Chin et al., 2002, Proc. Natl. Acad. Sci. USA, 99:11020-11024, Wang and Schultz, 2002, Chem. Comm., 1-10, and Chin and Schultz, 2002, ChemBioChem, 3:1135-1137. See also, International Patent Publications WO 2002/086075, entitled “Methods and Compositions for the Production of Orthogonal tRNA aminoacyl-tRNA Synthetase Pairs”; WO 2002/085923, entitled “In Vivo Incorporation of Unnatural Amino Acids”; and WO 2004/094593, entitled “Expanding the Eukaryotic Genetic Code”; and International Patent Application Number PCT US2004/022187, filed Jul. 7, 2004.

Concurrently, photoregulated peptides have been desired in order to study and manipulate biologically active molecules (e.g., enzymes). See, e.g., Adams, et al., Annu. Rev. Physiol., 1993, 55:755-784. To further expand the genetic code, there is a need to develop improved and/or additional components of the biosynthetic machinery, e.g., orthogonal tRNAs, orthogonal aminoacyl-tRNA synthetases and/or unique codons. There is a continuing need for novel methods to accomplish highly specific and targeted protein modifications as well as for the development of orthogonal translation components that incorporate unnatural amino acids in vivo into proteins at defined positions that allow the protein to be photoregulatable. This invention fulfills these and other needs, as will be apparent upon review of the following disclosure.

### SUMMARY OF THE INVENTION

The present invention comprises, inter alia, an orthogonal tRNA/synthetase pair for use in yeast (S. cerevisiae) based on the E. coli tRNA^{LEU}/leucyl tRNA-synthetase pair. Using a novel genetic selection, the invention identifies a series of synthetase mutants that selectively charge the amber suppressor tRNA with the C8 amino acid, α-aminocaprylic acid, α-methyl tyrosine, and/or the photocaged amino acid, o-nitrobenzyl cysteine, allowing them to be inserted into proteins in yeast in response to the amber nonsense codon, TAG. In other aspects, the present invention comprises an orthogonal tRNA/synthetase pair for use in E. coli based on the M. jannaschii tRNA^{Tyr}/tyrosyl tRNA-synthetase pair. Again, using novel genetic selection, the invention identifies synthetase mutants that selectively charge an O-tRNA with the photoregulatable (photoisomerizable) amino acid azobenzyl-Phe allowing them to be inserted into proteins in E. coli.

The present invention provides compositions and methods for the identification and isolation of aminoacyl-tRNA synthetase proteins that function in concert with a suitable tRNA to yield an orthogonal translation system for the incorporation of an unnatural amino acid of interest in vivo in a host cell.

In a certain aspect, the invention comprises a translation system which comprises an orthogonal tRNA (O-tRNA), or modified variant thereof, and, an orthogonal aminoacyl tRNA synthetase (O-RS) that preferentially charges the orthogonal tRNA, or modified variant thereof, with one or more amino acid. Such amino acid is selected from: α-aminocaprylic acid, o-nitrobenzyl cysteine, azobenzyl-Phe, and an O-RS or modified variant thereof, comprising a sequence of SEQ ID NO: 9-12, that preferentially charges the O-tRNA or modified variant thereof with O-methyl tyrosine. The translation systems herein can comprise a cell, e.g., a yeast cell such as S. cerevisiae or a eubacterial cell such as E. coli. Additionally, in various embodiments of the translation systems, the amino acid is an unnatural amino acid. In certain embodiments, the O-tRNA is a leucyl-O-tRNA, while in other embodiments, the O-tRNA is a tyrosyl-O-tRNA. Furthermore, in certain embodiments, the O-tRNA or modified variant thereof, the O-RS, or both the O-tRNA and the modified variant thereof, are derived from E. coli (e.g., from the wild-type E. coli-tRNA synthetase having the amino acid sequence of SEQ ID NO: 3), while in other embodiments, they are derived from M. jannaschii (e.g., from the wild-type M. jannaschii tRNA synthetase having the amino acid sequence of SEQ ID NO: 4). The translation systems herein can comprise O-RS (e.g., derived from the wild-type E. coli tRNA synthetase having the amino acid sequence of SEQ ID NO: 3), where the O-RS has an amino acid sequence that comprises (a) Ala, Val, His, Leu, Met, Phe, Gly, or Trp at amino acid position 40; (b) Ala, Met, Pro, Tyr, Glu, Trp, Ser, or Thr at amino acid position 41; (c) Pro, Leu, Ala, Arg, Ile, or Trp at amino acid position 499; (d) Val, Leu, Met, Ala, Phe, Cys, or Thr at amino acid position 527; and, (e) Gly at amino acid position 537. In other embodiments, the translation systems herein can comprise O-RS (e.g., that is derived from the wild-type M. jannaschii tRNA synthetase) having the amino acid sequence of SEQ ID NO: 4, where the O-RS has an amino acid sequence that comprises (a) Gly at amino acid position 32; (b) Glu at amino acid position 65; (c) Ala at amino acid position 108; (d) Glu at amino acid position 109; (e) Gly at amino acid position 158; and, (f) His at amino acid position 162. In other embodiments, the translation system has an O-RS that comprises an amino acid sequence selected from SEQ ID NO:5-17, and conservative variants thereof. In certain embodiments, the translation systems herein comprise comprises a polynucleotide encoding the O-RS (e.g., selected from the nucleotide sequences of SEQ ID NO:20-32), wherein the O-RS comprises an amino acid sequence selected from SEQ ID NO:5-17, and conservative variants thereof. The O-tRNA of the translation systems herein can comprise, or be encoded by, a polynucleotide sequence set forth in SEQ ID NO: 1-2. In some embodiments, the translation systems herein comprise a nucleic acid comprising a first O-RS and at least one selector codon that is recognized by a first O-tRNA. Such embodiments can further comprise a second O-RS and a second O-tRNA, wherein the second O-RS preferentially aminoacylates the second O-tRNA with a second amino acid that is different from the first amino acid, and wherein the second O-tRNA recognizes a selector codon that is different from the selector codon recognized by the first O-tRNA. The invention can also comprise translation systems wherein the O-tRNA, or modified variant thereof, comprises a recognition sequence for an amber codon and/or comprises a recognition sequence for TAG and/or comprises a target nucleic acid comprising an amber codon and/or comprises a protein encoded by the target nucleic acid. In some embodiments, the protein in the translation system comprises a photoregulated amino acid (e.g., an azobenzyl-Phe or an o-nitrobenzyl cysteine). The invention also includes proteins (e.g., unnatural amino acids such as α-aminocaprylic acid, o-nitrobenzyl cysteine, azobenzyl-Phe, or O-methyl tyrosine) produced by the translation systems herein, as well as compositions comprising such proteins.

In other aspects, the invention provides compositions comprising an orthogonal aminoacyl-tRNA synthetase (O-RS), that preferentially aminoacylates an O-tRNA with α-aminocaprylic acid, o-nitrobenzyl cysteine, or azobenzyl-Phe, or wherein the O-RS comprises the sequence of SEQ ID NO: 9-12, and preferentially aminoacylates an O-tRNA with O-methyl tyrosine. In such compositions, the O-tRNA can be a leucyl O-tRNA or a tyrosyl O-tRNA and can optionally recognize an amber selector codon (e.g., a TAG sequence), while the O-RS can be derived from E. coli or from M. jannaschii. The O-RS can comprise an amino acid sequence of SEQ ID NO: 5-17, or a conservative variation thereof and/or can preterentially aminoacylate the O-tRNA with an efficiency of at least 50% of the efficiency of any one of SEQ ID NO: 5-8 and 13-17. In some embodiments, the compositions of the invention comprise a cell (e.g., a yeast cell such as S. cerevisiae or a eubacterial cell such as E. coli), where the O-RS is encoded by one or more nucleic acids in the cell, where the nucleic acids are chosen from SEQ ID NO: 20-32 or a conservative variation thereof. The compositions of the invention can comprise a translation system. In embodiments wherein the compositions comprise a cell, the O-RS can be encoded by one or more nucleic acids in the cell; the cell can further comprise an orthogonal tRNA (O-tRNA) and, one or more of α-aminocaprylic acid, O-methyl tyrosine, o-nitrobenzyl cysteine, or azobenzyl-Phe; wherein the O-tRNA recognizes a selector codon, and the O-RS preferentially aminoacylates the O-tRNA with one of α-aminocaprylic acid, O-methyl tyrosine, o-nitrobenzyl cysteine, or azobenzyl-Phe. Such cells can comprises a target nucleic acid that encodes a polypeptide of interest, wherein the target nucleic acid comprises a selector codon that is recognized by the O-tRNA.

The invention also provides nucleic acids (e.g., chosen from SEQ ID NO: 20-32) that encode the amino acids of any of SEQ ID NO: 5-17, or conservative variations thereof.

In other aspects, the invention provides a protein comprising one or more of α-aminocaprylic acid, o-nitrobenzyl cysteine, or azobenzyl-Phe as well as compositions comprising such proteins.

In yet other embodiments, the invention comprises a method for selecting an active orthogonal aminoacyl-tRNA synthetase (O-RS) that charges an α-aminocaprylic acid, o-nitrobenzyl cysteine, or azobenzyl-Phe on an orthogonal tRNA (O-tRNA). Such methods comprise: subjecting a population of cells to selection, wherein the cells collectively comprise the O-tRNA (which is orthogonal to members of the population of cells that comprise the O-tRNA), a plurality of O-RS that comprises one or more active O-RS members that load the O-tRNA with an α-aminocaprylic acid, o-nitrobenzyl cysteine, or azobenzyl-Phe in one or more cells of the population; a polynucleotide that encodes a selectable marker (which polynucleotide comprises at least one selector codon that is recognized by the O-tRNA); and, α-aminocaprylic acid, o-nitrobenzyl cysteine, or azobenzyl-Phe; wherein a target cell in the population that comprises the active O-RS is identified by an enhanced suppression efficiency of the selectable marker as compared to a suppression efficiency of a control cell lacking the plurality of RS but comprising the O-tRNA; and, selecting the target cell, thereby selecting the active O-RS. In such selecting methods, the cells can additionally be selected in order to eliminate cells that comprise a non-target O-RS that charges the O-tRNA with an amino acid other than α-aminocaprylic acid, o-nitrobenzyl cysteine, or azobenzyl-Phe. Furthermore, in such selection methods, the selection can comprise a positive selection while the selectable marker an comprise a positive selection marker. In various embodiments, the O-tRNA can be a leucyl-O-tRNA or a tyrosyl-O-tRNA. The invention also includes an orthogonal aminoacyl-tRNA synthetase identified by such methods.

In other aspects, the invention comprises a method of producing a protein in a cell (e.g., one or more α-aminocaprylic acid, o-nitrobenzyl cysteine, azobenzyl-Phe, photoregulated serine, photoregulated serine analogue, fluorophore, spin labeled amino acid, or an amino acid comprising a dansyl side chain at one or more specified position). Such methods can comprise growing the cell (which comprises a nucleic acid that comprises at least one selector codon and that encodes a protein, as well as an orthogonal tRNA (O-tRNA) that recognizes the selector codon and an orthogonal aminoacyl-tRNA synthetase (O-RS) that preferentially aminoacylates the O-tRNA with the α-aminocaprylic acid, o-nitrobenzyl cysteine, azobenzyl-Phe, photoregulated serine, a photoregulated serine analogue, a fluorophore, a spin labeled amino acid, or an amino acid comprising a dansyl side chain) in an appropriate medium; and, providing α-aminocaprylic acid, o-nitrobenzyl cysteine, azobenzyl-Phe, photoregulated serine, a photoregulated serine analogue, a fluorophore, a spin labeled amino acid, or an amino acid comprising a dansyl side chain; incorporating the α-aminocaprylic acid, o-nitrobenzyl cysteine, azobenzyl-Phe, photoregulated serine, a photoregulated serine analogue, a fluorophore, a spin labeled amino acid, or an amino acid comprising a dansyl side chain into the specified position in response to the selector codon, thereby producing the protein. In such methods, the O-RS can comprise an amino acid sequence corresponding to SEQ ID NO: 5-17, or a conservative variation thereof.

The invention provides libraries of polynucleotides that can be used in screening for polynucleotides that encode preferred orthogonal aminoacyl-tRNA synthetase polypeptides (O-RS) that function in a host cell. These libraries comprise polynucleotides that encode variants of an amino acid sequence set forth in SEQ ID NO: 4 (the wild-type Methanococcus jannaschii tyrosyl-tRNA synthetase), where the library members have randomized nucleotide positions in codons encoding Tyr³², Leu⁶⁵, Phe¹⁰⁸, Gln¹⁰⁹, Asp¹⁵⁸ and Leu¹⁶² as numbered in SEQ.ID.NO: 4. Alternatively, the libraries comprise polynucleotides that encode variants of an Archaea aminoacyl-tRNA synthetase other than the amino acid sequence set forth in SEQ ID NO: 4 (e.g., an ortholog of the wild-type Methanococcus jannaschii tyrosyl-tRNA synthetase), where the polynucleotides have randomized nucleotide positions in codons spatially corresponding to Tyr³², Leu⁶¹, Phe¹⁰⁸, Gln¹⁰⁹, Asp¹⁵⁸ and Leu¹⁶² in the wild-type Methanococcus jannaschii tyrosyl-tRNA synthetase. In some embodiments, the polynucleotides in the library are cloned into an expression vector. In some embodiments, the O-RS preferentially aminoacylates an orthogonal tRNA (O-tRNA) with an unnatural amino acid. Any O-RS identified from the library can further have one or more conservative amino acid substitutions, for example, at positions other than the positions that were randomized. In some aspects, the libraries of the invention are within an E. coli host cell. In some embodiments, the Archaea aminoacyl-tRNA synthetase is a Methanococcus jannaschii aminoacyl-tRNA synthetase, or further, where the Methanococcus jannaschii aminoacyl-tRNA synthetase is a Methanococcus jannaschii tyrosyl-tRNA synthetase.

The invention also provides methods for screening libraries such as the libraries described above for the purpose of identifying a desired orthogonal aminoacyl-tRNA synthetase (O-RS) that incorporates an unnatural amino acid of interest. In some embodiments, these methods comprise the steps of providing (i) a library of polynucleotides encoding variants of the wild-type Methanococcus jannaschii tyrosyl-tRNA synthetase, where the polynucleotides have randomized nucleotide positions in codons encoding Tyr³², Leu⁶⁵, Phe¹⁰⁸, Gln¹⁰⁹, Asp¹⁵⁸ and Leu¹⁶²; and also providing a host cell, and the method comprising detecting a polynucleotide from the library that encodes an RS that preferentially aminoacylates an orthogonal tRNA (O-tRNA) with an unnatural amino acid in the host cell, thereby identifying a desired O-RS.

In some embodiments, the selection of the preferred variants typically entails a positive selection steps (e.g., expression of the chloramphenicol acetyltransferase protein and growth on chloramphenicol-containing media). In some embodiments, the positive selection is coupled with a negative selection step (e.g., counter selection of cells that express the toxic barnase protein).

In further aspects, the invention also provides further libraries of polynucleotides that can be used in screening for polynucleotides that encode preferred orthogonal aminoacyl-tRNA synthetase polypeptides (O-RS) that function in a host cell. Such libraries comprise polynucleotides that encode variants of an amino acid sequence set forth in SEQ ID NO: 3 (the wild-type Escherichia coli leucyl-tRNA synthetase), where the library members have randomized nucleotide positions in codons encoding Met⁴⁰, Leu⁴¹, Tyr⁴⁹⁹, Tyr⁵²⁷, and His⁵³⁷ as numbered in SEQ ID NO: 3. Alternatively, the libraries comprise polynucleotides that encode variants of a eubacterial aminoacyl-tRNA synthetase other than the amino acid sequence set forth in SEQ ID NO: 3 (e.g., an ortholog of the wild-type Escherichia coli leucyl-tRNA synthetase), where the polynucleotides have randomized nucleotide positions in codons spatially corresponding to Met⁴⁰, Leu⁴¹, Tyr⁴⁹⁹, Tyr⁵²⁷, and His⁵³⁷ in the wild-type Escherichia coli leucyl-tRNA synthetase. In some embodiments, the polynucleotides in the library are cloned into an expression vector. In some embodiments, the O-RS preferentially aminoacylates an orthogonal tRNA (O-tRNA) with an unnatural amino acid. Any O-RS identified from the library can further have one or more conservative amino acid substitutions, for example, at positions other than the positions that were randomized. In some aspects, the libraries of the invention are within a eukaryotic host cell such as S. cerevisiae. In some embodiments, the eubacterial aminoacyl-tRNA synthetase is a Escherichia coli aminoacyl-tRNA synthetase, or further, where the Escherichia coli aminoacyl-tRNA synthetase is a Escherichia coli leucyl-tRNA synthetase.

The invention also provides methods for screening libraries such as the libraries described above for the purpose of identifying a desired orthogonal aminoacyl-tRNA synthetase (O-RS) that incorporates an unnatural amino acid of interest. In some embodiments, these methods comprise the steps of providing (i) a library of polynucleotides encoding variants of the wild-type Methanococcus jannaschii tyrosyl-tRNA synthetase, where the polynucleotides have randomized nucleotide positions in codons encoding Tyr³², Leu⁶⁵ Phe¹⁰⁵, Gln¹⁰⁹, Asp¹⁵⁸ and Leu¹⁶² or a library of polynucleotides encoding variants of the wild-type Escherichia coli leucyl-tRNA synthetase, where the polynucleotides have randomized nucleotide positions in codons encoding Met⁴⁰, Leu⁴¹, Tyr⁴⁹⁹, Tyr⁵²⁷, and His⁵³⁷; and also providing a host cell, and the method comprising detecting a polynucleotide from the library that encodes an RS that preferentially aminoacylates an orthogonal tRNA (O-tRNA) with an unnatural amino acid in the host cell, thereby identifying a desired O-RS.

In some embodiments, the selection of the preferred variants typically entails a positive selection steps (e.g., expression of the chloramphenicol acetyltransferase protein and growth on chloramphenicol-containing media or expression of a gal4 gene product and growth on media lacking uracil or on media having aminotriazole but lacking histidine). In some embodiments, the positive selection is coupled with a negative selection step (e.g., counter selection of cells that express the toxic barnase protein or expression of a ura3 gene product in the presence of fluorootic acid.).

The invention also provides methods of modulating an activity of a protein, by incorporating an azobenzyl-Phe or o-nitrobenzyl cysteine into the protein via an O-RS and O-tRNA pair that are specific for azobenzyl-Phe or o-nitrobenzyl cysteine and exposing the protein to a wave length of light energy that photoregulates the azobenzyl-Phe or o-nitrobenzyl cysteine, thereby modulating the activity of the protein comprising the azobenzyl-Phe or o-nitrobenzyl cysteine. The invention also provides systems for modulating an activity of a protein. Such systems comprise a protein comprising azobenzyl-Phe or o-nitrobenzyl cysteine and a light source which photoregulates the azobenzyl-Phe or o-nitrobenzyl cysteine of the protein, thereby modulating the activity of the protein.

These and other objects and features of the invention will become more fully apparent when the following detailed description is read in conjunction with the accompanying figures.

### DEFINITIONS

Before describing the invention in detail, it is to be understood that this invention is not limited to particular biological systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the context clearly dictates otherwise. Thus, for example, reference to “a cell” includes a combination of two or more cells; reference to “bacteria” includes mixtures of bacteria, and the like.

Orthogonal: As used herein, the term “orthogonal” refers to a molecule (e.g., an orthogonal tRNA (O-tRNA) and/or an orthogonal aminoacyl tRNA synthetase (O-RS)) that functions with endogenous components of a cell with reduced efficiency as compared to a corresponding molecule that is endogenous to the cell or translation system, or that fails to function with endogenous components of the cell. In the context of tRNAs and aminoacyl-tRNA synthetases, orthogonal refers to an inability or reduced efficiency, e.g., less than 20% efficiency, less than 10% efficiency, less than 5% efficiency, or less than 1% efficiency, of an orthogonal tRNA to function with an endogenous tRNA synthetase compared to the ability of an endogenous tRNA to function with the endogenous tRNA synthetase; or of an orthogonal aminoacyl-tRNA synthetase to function with an endogenous tRNA compared to the ability of an endogenous tRNA synthetase to function with the endogenous tRNA. The orthogonal molecule lacks a functionally normal endogenous complementary molecule in the cell. For example, an orthogonal tRNA in a cell is aminoacylated by any endogenous tRNA synthetase (RS) of the cell with reduced or even undetectable or zero efficiency, when compared to aminoacylation of an endogenous tRNA by the endogenous RS. In another example, an orthogonal RS aminoacylates any endogenous tRNA in a cell of interest with reduced or even undetectable or zero efficiency, as compared to aminoacylation of the endogenous tRNA by an endogenous RS. A second orthogonal molecule can be introduced into the cell that functions with the first orthogonal molecule. For example, an orthogonal tRNA/RS pair includes introduced complementary components that function together in the cell with an efficiency (e.g., 45% efficiency, 50% efficiency, 60% efficiency, 70% efficiency, 75% efficiency, 80% efficiency, 90% efficiency, 95% efficiency, or 99% or more efficiency) as compared to that of a control, e.g., a corresponding tRNA/RS endogenous pair, or an active orthogonal pair (e.g., a leucyl orthogonal tRNA/RS pair).

Orthogonal leucyl-tRNA: As used herein, an orthogonal leucyl-tRNA (leucyl-O-tRNA) is a tRNA that is orthogonal to a translation system of interest, where the tRNA is: (1) identical or substantially similar to a naturally occurring leucyl-tRNA; (2) derived from a naturally occurring leucyl-tRNA by natural or artificial mutagenesis; (3) derived by any process that takes a sequence of a wild-type or mutant leucyl-tRNA sequence of (1) or (2) into account; (4) homologous to a wild-type or mutant leucyl-tRNA; (5) homologous to any example tRNA that is designated as a substrate for a leucyl-tRNA synthetase in Table 2 or 3; or, (6) a conservative variant of any example tRNA that is designated as a substrate for a leucyl-tRNA synthetase in Table 2 or 3. The leucyl-tRNA can exist charged with an amino acid, or in an uncharged state. It is also to be understood that a “leucyl-O-tRNA” optionally is charged (aminoacylated) by a cognate synthetase with an amino acid other than leucine, e.g., with OMe-L-tyrosine, α-aminocaprylic acid, or a photoregulated amino acid such as o-nitrobenzyl cysteine. Indeed, it will be appreciated that a leucyl-O-tRNA of the invention is advantageously used to insert essentially any amino acid (e.g., OMe-L-tyrosine, α-aminocaprylic acid, or a photoregulated amino acid such as o-nitrobenzyl cysteine), whether natural or artificial, into a growing polypeptide, during translation, in response to a selector codon.

Orthogonal tyrosyl-tRNA: As used herein, an orthogonal tyrosyl-tRNA (tyrosyl-O-tRNA) is a tRNA that is orthogonal to a translation system of interest, where the tRNA is: (1) identical or substantially similar to a naturally occurring tyrosyl-tRNA; (2) derived from a naturally occurring tyrosyl-tRNA by natural or artificial mutagenesis; (3) derived by any process that takes a sequence of a wild-type or mutant tyrosyl-tRNA sequence of (1) or (2) into account; (4) homologous to a wild-type or mutant tyrosyl-tRNA; (5) homologous to any example tRNA that is designated as a substrate for a tyrosyl-tRNA synthetase in the examples or sequence listing herein, e.g., azobenzyl-Phe; or, (6) a conservative variant of any example tRNA that is designated as a substrate for a tyrosyl-tRNA synthetase in the examples or sequence listing herein. The tyrosyl-tRNA can exist charged with an amino acid, or in an uncharged state. It is also to be understood that a “tyrosyl-O-tRNA” optionally is charged (aminoacylated) by a cognate synthetase with an amino acid other than tyrosine, e.g., with a photoregulated amino acid such as azobenzyl-Phe. Indeed, it will be appreciated that a tyrosyl-O-tRNA of the invention is advantageously used to insert essentially any amino acid (e.g., a photoregulated amino acid such as azobenzyl-Phe), whether natural or artificial, into a growing polypeptide, during translation, in response to a selector codon.

Orthogonal leucyl-amino acid synthetase: As used herein, an orthogonal leucyl amino acid synthetase (leucyl-O-RS) is an enzyme that preferentially aminoacylates the leucyl-O-tRNA with an amino acid in a translation system of interest. The amino acid that the leucyl-O-RS charges (or loads) onto the leucyl-O-tRNA can be any amino acid, whether natural or artificial, and is not necessarily limited herein. In certain embodiments, the amino acid is OMe-L-tyrosine, α-aminocaprylic acid, or a photoregulated amino acid such as o-nitrobenzyl cysteine. The synthetase is optionally the same as, or homologous to, a naturally occurring leucyl amino acid synthetase, or the same as or homologous to a synthetase designated as a leucyl-O-RS in Table 2 or 3 or the examples and sequence listing herein. For example, the leucyl-O-RS can be a conservative variant of a leucyl-O-RS of Table 2 or 3 or from the examples or sequence listing herein, and/or can be at least 50%, 60%, 70%, 80%, 90%, 95%, 98%, 99% or more identical in sequence to a leucyl-O-RS of Table 2 or 3 or from the examples or sequence listing herein.

Orthogonal tyrosyl-amino acid synthetase: As used herein, an orthogonal tyrosyl amino acid synthetase (tyrosyl-O-RS) is an enzyme that preferentially aminoacylates the tyrosyl-O-tRNA with an amino acid in a translation system of interest. The amino acid that the tyrosyl-O-RS loads onto the tyrosyl-O-tRNA can be any amino acid, whether natural or artificial, and is not necessarily limited herein. In certain embodiments, the amino acid is a photoregulated amino acid such as azobenzyl-Phe. The synthetase is optionally the same as, or homologous to, a naturally occurring tyrosyl amino acid synthetase, or the same as or homologous to a synthetase designated as a tyrosyl-O-RS in the examples or sequence listing herein. For example, the tyrosyl-O-RS can be a conservative variant of a tyrosyl-O-RS of the examples or sequence listing herein, and/or can be at least 50%, 60%, 70%, 80%, 90%, 95%, 98%, 99% or more identical in sequence to a tyrosyl-O-RS from the examples or sequence listing herein.

Cognate: The term “cognate” refers to components that function together, e.g., an orthogonal tRNA and an orthogonal aminoacyl-tRNA synthetase. The components can also be referred to as being “complementary.”

Preferentially aminoacylates: As used herein in reference to orthogonal translation systems, an O-RS “preferentially aminoacylates” a cognate O-tRNA when the O-RS charges the O-tRNA with an amino acid more efficiently than it charges any endogenous tRNA in an expression system. That is, when the O-tRNA and any given endogenous tRNA are present in a translation system in approximately equal molar ratios, the O-RS will charge the O-tRNA more frequently than it will charge the endogenous tRNA. Preferably, the relative ratio of O-tRNA charged by the O-RS to endogenous tRNA charged by the O-RS is high, preferably resulting in the O-RS charging the O-tRNA exclusively, or nearly exclusively, when the O-tRNA and endogenous tRNA are present in equal molar concentrations in the translation system. The relative ratio between O-tRNA and endogenous tRNA that is charged by the O-RS, when the O-tRNA and O-RS are present at equal molar concentrations, is greater than 1:1, preferably at least about 2:1, more preferably 5; 1, still more preferably 10:1, yet more preferably 20; 1, still more preferably 50:1, yet more preferably 75:1, still more preferably 95; 1, 98:1, 99:1, 100:1, 500:1, 1,000:1, 5,000:1 or higher.

As used herein, the phrase “amino acid positions that spatially correspond” and similar expressions refer to amino acid positions in two orthologous proteins, where the two positions are functionally identical, but where the positions do not reside in identical locations in the primary protein structures (i.e., the two orthologous proteins do not have identical amino acid sequences).

Selector codon: The term “selector codon” refers to a codon recognized by an O-tRNA in a translation process that is not typically recognized by an endogenous tRNA. An O-tRNA anticodon loop recognizes a selector codon, e.g., on an mRNA, and inserts its amino acid (e.g., an unnatural amino acid such as OMe-L-tyrosine, α-aminocaprylic acid, or a photoregulated amino acid such as o-nitrobenzyl cysteine or azobenzyl-Phe) at this site in the polypeptide being translated. For example, in one embodiment herein, the O-tRNA recognizes a selector codon such as an amber codon and adds an unnatural amino acid, such as OMe-L-tyrosine, α-aminocaprylic acid, or a photoregulated amino acid such as o-nitrobenzyl cysteine or azobenzyl-Phe, into a polypeptide being produced by the translation process. Selector codons can include, e.g., nonsense codons, such as stop codons, e.g., amber, ochre, and opal codons; four or more base codons; rare codons; codons derived from natural or unnatural base pairs and/or the like.

Suppressor tRNA: A suppressor tRNA is a tRNA that alters the reading of a messenger RNA (mRNA) in a given translation system, e.g., by providing a mechanism for incorporating an amino acid into a polypeptide chain in response to a selector codon. For example, a suppressor tRNA can read through, e.g., a stop codon (such as an amber, ocher, or opal codon), a four base codon, a rare codon, etc.

Suppression activity: As used herein, the term “suppression activity” refers, in general, to the ability of a tRNA (e.g., a suppressor tRNA) to allow translational read-through of a codon (e.g. a selector codon that is a stop codon such as an amber codon, or a 4-or-more base codon) that would otherwise result in the termination of translation or mistranslation (e.g., frame-shifting). Suppression activity of a suppressor tRNA can be expressed as a percentage of translational read-through activity observed compared to a second suppressor tRNA, or as compared to a control system, e.g., a control system lacking an O-RS.

The present invention provides various means by which suppression activity can be quantified. Percent suppression of a particular O-tRNA and O-RS against a selector codon (e.g., an amber codon) of interest refers to the percentage of activity of a given expressed test marker (e.g., LacZ), that includes a selector codon, in a nucleic acid encoding the expressed test marker in a translation system of interest, where the translation system of interest includes an O-RS and an O-tRNA. This is as compared to a positive control construct, where the positive control lacks the O-tRNA, the O-RS and the selector codon. Thus, for example, if an active positive control marker construct that lacks a selector codon has an observed activity of “X” in a given translation system, in units relevant to the marker assay at issue, then percent suppression of a test construct comprising the selector codon is the percentage of “X” that the test marker construct displays under essentially the same environmental conditions as the positive control marker was expressed under, except that the test marker construct is expressed in a translation system that also includes the O-tRNA and the O-RS. Typically, the translation system expressing the test marker also includes an amino acid that is recognized by the O-RS and O-tRNA. Optionally, the percent suppression measurement can be refined by comparison of the test marker to a “background” or “negative” control marker construct, which includes the same selector codon as the test marker, but in a system that does not include the O-tRNA, O-RS and/or relevant amino acid recognized by the O-tRNA and/or O-RS. This negative control is useful in normalizing percent suppression measurements to account for background signal effects from the marker in the translation system of interest.

Suppression efficiency can be determined by any of a number of assays known in the art. For example, a β-galactosidase reporter assay can be used, e.g., a derivatized lacZ plasmid (where the construct has a selector codon in the lacZ nucleic acid sequence) is introduced into cells from an appropriate organism (e.g., an organism where the orthogonal components can be used) along with plasmid comprising an O-tRNA of the invention. A cognate synthetase can also be introduced (either as a polypeptide or a polynucleotide that encodes the cognate synthetase when expressed). The cells are grown in media to a desired density, e.g., to an OD₆₀₀ of about 0.5, and β-galactosidase assays are performed, e.g., using the BetaFluor™ β-Galactosidase Assay Kit (Novagen, San Diego, Calif.). Percent suppression can be calculated as the percentage of activity for a sample relative to a comparable control, e.g., the value observed from the derivatized lacZ construct, where the construct has a corresponding sense codon at desired position rather than a selector codon.

Translation system: The term “translation system” refers to the components that incorporate an amino acid into a growing polypeptide chain (protein). Components of a translation system can include, e.g., ribosomes, tRNAs, synthetases, mRNA and the like. The O-tRNA and/or the O-RSs of the invention can be added to, or be part of, an in vitro or in vivo translation system, e.g., in a prokaryotic (non-eukaryotic) cell, e.g., a bacterium (such as E. coli), or in a eukaryotic cell, e.g., a yeast cell such as S. cerevisiae, a mammalian cell, a plant cell, an algae cell, a fungus cell, an insect cell, and/or the like.

Unnatural amino acid: As used herein, the term “unnatural amino acid” refers to any amino acid, modified amino acid, and/or amino acid analogue, such as OMe-L-tyrosine, α-aminocaprylic acid, or a photoregulated amino acid such as o-nitrobenzyl cysteine or azobenzyl-Phe, that is not one of the 20 common naturally occurring amino acids or the rare natural amino acids seleno cysteine or pyrolysine.

Derived from: As used herein, the term “derived from” refers to a component that is isolated from or made using a specified molecule or organism, or information from the specified molecule or organism. For example, a polypeptide that is derived from a second polypeptide comprises an amino acid sequence that is identical or substantially similar to the amino acid sequence of the second polypeptide. In the case of polypeptides, the derived species can be obtained by, for example, naturally occurring mutagenesis, artificial directed mutagenesis or artificial random mutagenesis. The mutagenesis used to derive polypeptides can be intentionally directed or intentionally random. The mutagenesis of a polypeptide to create a different polypeptide derived from the first can be a random event (e.g., caused by polymerase infidelity) and the identification of the derived polypeptide can be serendipitous. Mutagenesis of a polypeptide typically entails manipulation of the polynucleotide that encodes the polypeptide.

Positive selection or screening marker: As used herein, the term “positive selection or screening marker” refers to a marker that when present, e.g., expressed, activated, or the like, results in identification of a cell that comprises a trait corresponding to the marker, e.g., cells with the positive selection marker, from those without the trait.

Negative selection or screening marker: As used herein, the term “negative selection or screening marker” refers to a marker that, when present, e.g., expressed, activated, or the like, allows identification of a cell that does not comprise a selected property or trait (e.g., as compared to a cell that does possess the property or trait).

Reporter: As used herein, the term “reporter” refers to a component that can be used to identify and/or select target components of a system of interest. For example, a reporter can include a protein, e.g., an enzyme, that confers antibiotic resistance or sensitivity (e.g., β-lactamase, chloramphenicol acetyltransferase (CAT), and the like), a fluorescent screening marker (e.g., green fluorescent protein, YFP, EGFP, RFP, etc.), a luminescent marker (e.g., a firefly luciferase protein), an affinity based screening marker, or positive or negative selectable marker genes such as lacZ, β-gal/lacZ (β-galactosidase), ADH (alcohol dehydrogenase), his3, ura3, leu2, lys2, or the like.

Eukaryote: As used herein, the term “eukaryote” refers to organisms belonging to the phylogenetic domain, or Superkingdom, Eucarya. Eukaryotes are generally distinguishable from prokaryotes by their typically multicellular organization (however, some eukaryotes such as yeast are not typically multicellular), the presence of a membrane-bound nucleus and other membrane-bound organelles, linear genetic material (i.e., linear chromosomes), the absence of operons, the presence of introns, message capping and poly-A mRNA, and other biochemical characteristics, such as a distinguishing ribosomal structure. Eukaryotic organisms include, for example, animals (e.g., mammals, insects, reptiles, birds, etc.), ciliates, plants (e.g., monocots, dicots, algae, etc.), fungi, yeasts, flagellates, microsporidia, protists, etc.

Prokaryote: As used herein, the term “prokaryote” refers to non-eukaryotic organisms, e.g., belonging to the domains, or superkingdoms, of Eubacteria and Archaea, and sometimes referred to as Monera. Prokaryotic organisms are generally distinguishable from eukaryotes by their unicellular organization, asexual reproduction by budding or fission, the lack of a membrane-bound nucleus or other membrane-bound organelles, a circular chromosome, the presence of operons, the absence of introns, message capping and poly-A mRNA, and other biochemical characteristics, such as a distinguishing ribosomal structure. The Prokarya include both Eubacteria and Archaea, or Archaeabacteria. Cyanobacteria (the blue green algae) and mycoplasma can also be included within this classification.

Bacteria and Archaea: As used herein, the terms “bacteria” and eubacteria” refer to prokaryotic organisms that are distinguishable from “Archaea.” Similarly, Archaea refers to prokaryotes that are distinguishable from eubacteria. Eubacteria and Archaea can be distinguished by a number of morphological and biochemical criteria. For example, differences in ribosomal RNA sequences, RNA polymerase structure, the presence or absence of introns, antibiotic sensitivity, the presence or absence of cell wall peptidoglycans and other cell wall components, the branched versus unbranched structures of membrane lipids, and the presence/absence of histones and histone-like proteins are used to differentiate between Eubacteria and Archaea.

Examples of Eubacteria include, e.g., Escherichia coli, Thermus thermophilus, Bacillus stearothermophilus. Examples of Archaea include, e.g., Methanococcus jannaschii (Mj), Methanosarcina mazei (Mm), Methanobacterium thernioautotrophicum (Mt), Methanococcus maripaludis, Methanopyrus kandleri, Halobacterium such as Haloferax volcanii and Halobacterium species NRC-1, Archaeoglobus fulgidus (Af), Pyrococcus furiosus (Pf), Pyrococcus horikoshii (Ph), Pyrobaculum aerophilum, Pyrococcus abyssi, Sulfolobus solfataricus (Ss), Sulfolobus tokodaii, Aeuropyrum pernix (Ap), Thermoplasma acidophilum, and Thermoplasma volcanium.

Conservative variant: As used herein, the term “conservative variant,” in the context of a translation component, refers to a translation component, e.g., a conservative variant O-tRNA or a conservative variant O-RS, that functionally performs similar to a base component that the conservative variant is similar to, e.g., an O-tRNA or O-RS, having variations in the sequence as compared to a reference O-tRNA or O-RS. For example, an O-RS will aminoacylate a complementary O-tRNA or a conservative variant O-tRNA with an unnatural amino acid, e.g., OMe-L-tyrosine, α-aminocaprylic acid, or a photoregulated amino acid such as o-nitrobenzyl cysteine or azobenzyl-Phe, although the O-tRNA and the conservative variant O-tRNA do not have the same sequence. The conservative variant can have, e.g., one variation, two variations, three variations, four variations, or five or more variations in sequence, as long as the conservative variant is complementary to the corresponding O-tRNA or O-RS.

Selection or screening agent: As used herein, the term “selection or screening agent” refers to an agent that, when present, allows for selection/screening of certain components from a population. For example, a selection or screening agent can be, but is not limited to, e.g., a nutrient, an antibiotic, a wavelength of light, an antibody, an expressed polynucleotide, or the like. The selection agent can be varied, e.g., by concentration, intensity, etc.

In response to: As used herein, the term “in response to” refers to the process in which an O-tRNA of the invention recognizes a selector codon and mediates the incorporation of the relevant amino acid, e.g., an unnatural amino acid such as OMe-L-tyrosine, α-aminocaprylic acid, or a photoregulated amino acid such as o-nitrobenzyl cysteine or azobenzyl-Phe, which is coupled to the tRNA, into the growing polypeptide chain.

Encode: As used herein, the term “encode” refers to any process whereby the information in a polymeric macromolecule or sequence string is used to direct the production of a second molecule or sequence string that is different from the first molecule or sequence string. As used herein, the term is used broadly, and can have a variety of applications. In one aspect, the term “encode” describes the process of semi-conservative DNA replication, where one strand of a double-stranded DNA molecule is used as a template to encode a newly synthesized complementary sister strand by a DNA-dependent DNA polymerase.

In another aspect, the term “encode” refers to any process whereby the information in one molecule is used to direct the production of a second molecule that has a different chemical nature from the first molecule. For example, a DNA molecule can encode an RNA molecule (e.g., by the process of transcription incorporating a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

### BRIEF DESCRIPTION OF THE FIGURES

FIG. 1, Panels A-C shows: (A) Primary sequence of the leucyl suppressor tRNA, Leu5_{CUA}; (B) The ttLRS active site with a bound leucyl sulfamoyl adenylate inhibitor (100, PDB entry 1H3N); (C) Structures of O-methyl tyrosine (also written as OMe-L-tyrosine), α-aminocaprylic acid and o-nitrobenzyl cysteine.

FIG. 2, Panels A-B shows: (A) Expression of hSOD-33TAG-His6 in the presence (+lanes) and absence (−lanes) of 1 mM unnatural amino acids. (B) Caspase 3 activity in an untreated cell lysate (nbC), after irradiation (nbC/UV), after irradiation in presence of granzyme B (nbC/UV/granzyme B), and in the presence of a caspase 3 inhibitor (nbC/Inh).

FIG. 3 shows comparison between an inactive photocaged enzyme (o-nitrobenzyl cysteine) and a functional cysteine enzyme after irradiation.

FIG. 4, Panels A and B, schematically shows conversion of trans azobenzyl-Phe and cis azobenzyl-Phe (Panel A), and the synthesis of azobenzyl-Phe (Panel B).

FIG. 5, displays the spectral characterization of genetically encoded azobenzyl-Phe containing mutant CAP.

FIG. 6, shows a gel-mobility shift assay to determine CAP (wild-type or mutant CAP70TAG; 160 nM) binding to the lactose promoter fragment.

FIG. 7, shows characterization of the genetically encoded azobenzyl-Phe containing mutant proteins.

FIG. 8, displays the ESI mass spectrum of mutant myoglobin expressed with the photoregulatable azobenzyl-Phe at position 75.

FIG. 9, panels A through G, show HPLC analysis of PTH-amino acid standards of the natural 20 amino acids (panel A); and the protein sequence of mutant myoglobin (for Myo4TAG) for the first 6 amino acids from the N-terminus.

FIG. 10, shows a gel assay of serial dilutions of CAP (wild-type and mutant, photoswitched or unswitched) at constant promoter (33 nM) for determination of binding constants using linear regression.

FIG. 11, shows EMSA method of determination of CAP binding affinity for primary lac binding site.

### DETAILED DESCRIPTION

In order to add additional synthetic amino acids, such as OMe-L-tyrosine, α-aminocaprylic acid, or a photoregulated amino acid such as o-nitrobenzyl cysteine or azobenzyl-Phe to the genetic code, in vivo, new orthogonal pairs of an aminoacyl-tRNA synthetase and a tRNA are needed that can function efficiently in the translational machinery, but that are “orthogonal,” to the translation system at issue, meaning that the pairs function independently of the synthetases and tRNAs endogenous to the translation system. Desired characteristics of such orthologous pair include tRNA that decode or recognize only a specific new codon, e.g., a selector codon, that is not decoded by any endogenous tRNA, and aminoacyl-tRNA synthetases that preferentially aminoacylate (or charge) its cognate tRNA with only a specific non-natural amino acid, e.g., OMe-L-tyrosine, α-aminocaprylic acid, or a photoregulated amino acid such as o-nitrobenzyl cysteine or azobenzyl-Phe. The O-tRNA is also desirably not aminoacylated by endogenous synthetases. For example, in E. coli, an orthogonal pair will include an aminoacyl-tRNA synthetase that does not substantially aminoacylate any of the endogenous tRNAs, e.g., of which there are 40 in E. coli, and an orthogonal tRNA that is not aminoacylated by any of the endogenous synthetases, e.g., of which there are 21 in E. coli.

The invention comprises the generation of new orthogonal synthetase/tRNA pairs derived from E. coli tRNA^{Leu} sequences that efficiently and selectively incorporate photoregulated amino acids (e.g., o-nitrobenzyl cysteine), O-Me-L-tyrosine, and α-aminocaprylic acid in yeast into proteins such as caspase 3 in response to the three-base selector codon TAG. The invention also comprises the generation of new orthogonal synthetase/tRNA pairs derived from M. jannaschii tRNA^{Tyr} sequences that efficiently and selectively incorporate the photoregulated amino acid azobenzyl-Phe in E. coli into proteins such as CAP in response to the three base selector codon TAG.

In order to encode unnatural amino acids in vivo, one has to generate an orthogonal tRNA (O-tRNA) that uniquely recognizes specific codon and a corresponding synthetase that uniquely aminoacylates only this O-tRNA with an unnatural amino acid of interest.

This invention provides libraries, compositions of and methods for identifying and producing additional orthogonal tRNA-aminoacyl-tRNA synthetase pairs, e.g., O-tRNA/O-RS pairs that can be used to incorporate unnatural amino acids, e.g., OMe-L-tyrosine, α-aminocaprylic acid, or a photoregulated amino acid such as o-nitrobenzyl cysteine or azobenzyl-Phe. An example O-tRNA of the invention is capable of mediating incorporation of OMe-L-tyrosine, α-aminocaprylic acid, or a photoregulated amino acid such as o-nitrobenzyl cysteine or azobenzyl-Phe into a protein that is encoded by a polynucleotide, which comprises a selector codon that is recognized by the O-tRNA, e.g., in vivo. The anticodon loop of the O-tRNA recognizes the selector codon on an mRNA and incorporates its amino acid, e.g., OMe-L-tyrosine, α-aminocaprylic acid, or a photoregulated amino acid such as o-nitrobenzyl cysteine or azobenzyl-Phe at this site in the polypeptide. An orthogonal aminoacyl-tRNA synthetase of the invention preferentially aminoacylates (or charges) its O-tRNA with only a specific unnatural amino acid.

Orthogonal tRNA/orthogonal aminoacyl-tRNA synthetases and Pairs Thereof

An orthogonal pair is composed of an O-tRNA, e.g., a suppressor tRNA, a frameshift tRNA, or the like, and an O-RS. The O-tRNA is not acylated by endogenous synthetases and is capable of mediating incorporation of an unnatural amino acid (e.g., an O-Me-L-tyrosine, α-aminocaprylic acid, or photoregulated amino acid such as o-nitrobenzyl cysteine or azobenzyl-Phe) into a protein that is encoded by a polynucleotide that comprises a selector codon that is recognized by the O-tRNA in vivo or in vitro. The O-RS recognizes the O-tRNA and preferentially aminoacylates the O-tRNA with an unnatural amino acid, e.g., in a eukaryotic cell, a prokaryotic cell, in vitro, etc. Methods for producing orthogonal pairs along with orthogonal pairs produced by such methods and compositions of orthogonal pairs for use are included in the invention. The development of multiple orthogonal tRNA/synthetase pairs can allow the simultaneous incorporation of multiple unnatural amino acids using different codons. International Publication Numbers WO 2002/086075, entitled “METHODS AND COMPOSITION FOR THE PRODUCTION OF ORTHOGONAL tRNA-AMINOACYL-tRNA SYNTHETASE PAIRS,” WO 2002/085923, entitled “IN VIVO INCORPORATION OF UNNATURAL AMINO ACIDS,” and WO 2004/094593, entitled “EXPANDING THE EUKARYOTIC GENETIC CODE” describe translation systems amenable to use with, or by, the current invention. In addition, see International Application Number PCT/US2004/011786, filed Apr. 16, 2004 and International Application Number PCT/US2004/022187, filed Jul. 7, 2004. Each of these applications is incorporated herein by reference in its entirety. Translation systems generally comprise cells (which can be non-eukaryotic cells such as E. coli, or eukaryotic cells such as yeast, e.g., S. cerevisiae) that include an orthogonal tRNA (O-tRNA), an orthogonal aminoacyl tRNA synthetase (O-RS), and an unnatural amino acid. In the present invention, these systems are adapted for incorporation of unnatural amino acids such as OMe-L-tyrosine, α-aminocaprylic acid, or a photoregulated amino acid such as o-nitrobenzyl cysteine or azobenzyl-Phe. The O-RS aminoacylates the O-tRNA with the OMe-L-tyrosine, α-aminocaprylic acid, or photoregulated amino acid such as o-nitrobenzyl cysteine or azobenzyl-Phe. An orthogonal pair of the invention includes an O-tRNA, e.g., a suppressor tRNA, a frameshift tRNA, or the like, and an O-RS. Individual components are also provided in the invention.

In general, when an orthogonal pair recognizes a selector codon and loads an amino acid in response to the selector codon, the orthogonal pair is said to “suppress” the selector codon. That is, a selector codon that is not recognized by the translation system's (e.g., cell's) endogenous machinery is not ordinarily translated, which can result in blocking production of a polypeptide that would otherwise be translated from the nucleic acid. An O-tRNA of the invention recognizes a selector codon and includes at least about, e.g., a 45%, a 50%, a 60%, a 75%, a 80%, or a 90% or more suppression efficiency in the presence of a cognate synthetase in response to a selector codon as compared to an O-tRNA comprising or encoded by a polynucleotide sequence as set forth herein (e.g., in the Sequence Listing). The O-RS aminoacylates the O-tRNA with an unnatural amino acid of interest, such as OMe-L-tyrosine, α-aminocaprylic acid, or a photoregulated amino acid such as o-nitrobenzyl cysteine or azobenzyl-Phe. The cell uses the O-tRNA/O-RS pair to incorporate the unnatural amino acid into a growing polypeptide chain, e.g., via a nucleic acid that comprises a polynucleotide that encodes a polypeptide of interest, where the polynucleotide comprises a selector codon that is recognized by the O-tRNA. In certain desirable aspects, the cell can include an additional O-tRNA/O-RS pair, where the additional O-tRNA is loaded by the additional O-RS with a different unnatural amino acid. For example, one of the O-tRNAs can recognize a four base codon and the other can recognize a stop codon. Alternately, multiple different stop codons or multiple different four base codons can specifically recognize different selector codons.

In certain embodiments, the invention comprises a cell such as an E. coli cell or an S. cerevisiae cell that includes an orthogonal tRNA (O-tRNA), an orthogonal aminoacyl-tRNA synthetase (O-RS), an unnatural amino acid, e.g., OMe-L-tyrosine, α-aminocaprylic acid, or a photoregulated amino acid such as o-nitrobenzyl cysteine or azobenzyl-Phe, and a nucleic acid that comprises a polynucleotide that encodes a polypeptide of interest, where the polynucleotide comprises the selector codon that is recognized by the O-tRNA. The translation system can also be a cell-free system, e.g., any of a variety of commercially available “in vitro” transcription/translation systems in combination with an O-tRNA/ORS pair and an unnatural amino acid as described herein.

In one embodiment, the suppression efficiency of the O-RS and the O-tRNA together is about, e.g., 5 fold, 10 fold, 15 fold, 20 fold, or 25 fold or more greater than the suppression efficiency of the O-tRNA lacking the O-RS. In one aspect, the suppression efficiency of the O-RS and the O-tRNA together is at least about, e.g., 35%, 40%, 45%, 50%, 60%, 75%, 80%, or 90% or more of the suppression efficiency of an orthogonal synthetase pair as set forth herein (e.g., in the Sequence Listing or examples).

As noted, the invention optionally includes multiple O-tRNA/O-RS pairs in a cell or other translation system, which allows incorporation of more than one unnatural amino acid, e.g., OMe-L-tyrosine, α-aminocaprylic acid, or a photoregulated amino acid such as o-nitrobenzyl cysteine or azobenzyl-Phe and another unnatural amino acid. For example, the cell can further include an additional different O-tRNA/O-RS pair and a second unnatural amino acid, where this additional O-tRNA recognizes a second selector codon and this additional O-RS preferentially aminoacylates the O-tRNA with the second unnatural amino acid. For example, a cell that includes an O-tRNA/O-RS pair (where the O-tRNA recognizes, e.g., an amber selector codon), can further comprise a second orthogonal pair, e.g., leucyl, lysyl, glutamyl, etc., (where the second O-tRNA recognizes a different selector codon, e.g., an opal, four-base codon, or the like). Desirably, the different orthogonal pairs are derived from different sources, which can facilitate recognition of different selector codons.

The O-tRNA and/or the O-RS can be naturally occurring or can be, e.g., derived by mutation of a naturally occurring tRNA and/or RS, e.g., by generating libraries of tRNAs and/or libraries of RSs, from any of a variety of organisms and/or by using any of a variety of available mutation strategies. For example, one strategy for producing an orthogonal tRNA/aminoacyl-tRNA synthetase pair involves importing a heterologous (to the host cell) tRNA/synthetase pair from, e.g., a source other than the host cell, or multiple sources, into the host cell. The properties of the heterologous synthetase candidate include, e.g., that it does not charge any host cell tRNA, and the properties of the heterologous tRNA candidate include, e.g., that it is not aminoacylated by any host cell synthetase. In addition, the heterologous tRNA is orthogonal to all host cell synthetases.

A second strategy for generating an orthogonal pair involves generating mutant libraries from which to screen and/or select an O-tRNA or O-RS. See Examples below. These strategies can also be combined.

Orthogonal tRNA (O-tRNA)

An orthogonal tRNA (O-tRNA) of the invention desirably mediates incorporation of an unnatural amino acid, such as OMe-L-tyrosine, α-aminocaprylic acid, or a photoregulated amino acid such as o-nitrobenzyl cysteine or azobenzyl-Phe into a protein that is encoded by a polynucleotide that comprises a selector codon (e.g., an amber codon) that is recognized by the O-tRNA, e.g., in vivo or in vitro. In certain embodiments, an O-tRNA of the invention includes at least about, e.g., a 45%, a 50%, a 60%, a 75%, a 80%, or a 90% or more suppression efficiency in the presence of a cognate synthetase in response to a selector codon as compared to an O-tRNA comprising or encoded by a polynucleotide sequence as set forth in the O-tRNA sequences in the sequences herein (e.g., in the Sequence Listing and/examples).

Suppression efficiency can be determined by any of a number of assays known in the art. For example, a β-galactosidase reporter assay can be used, e.g., a derivatized lacZ plasmid (where the construct has a selector codon in the lacZ nucleic acid sequence) is introduced into cells from an appropriate organism (e.g., an organism where the orthogonal components can be used) along with plasmid comprising an O-tRNA of the invention. A cognate synthetase can also be introduced (either as a polypeptide or a polynucleotide that encodes the cognate synthetase when expressed). The cells are grown in media to a desired density, e.g., to an OD₆₀₀ of about 0.5, and β-galactosidase assays are performed, e.g., using the BetaFluor™ β-Galactosidase Assay Kit (Novagen, San Diego, Calif.). Percent suppression can be calculated as the percentage of activity for a sample relative to a comparable control, e.g., the value observed from the derivatized lacZ construct, where the construct has a corresponding sense codon at desired position rather than a selector codon.

Examples of O-tRNAs of the invention are set forth herein (e.g., in the Sequence Listing). See also, the tables, examples and figures herein for sequences of exemplary O-tRNA and O-RS molecules. See also, the section entitled “Nucleic Acid and Polypeptide Sequence and Variants” herein. In an RNA molecule, such as an O-RS mRNA, or O-tRNA molecule, Thymine (T) is replace with Uracil (U) relative to a given sequence (or vice versa for a coding DNA), or complement thereof. Additional modifications to the bases can also be present.

The invention also includes conservative variations of O-tRNAs corresponding to particular O-tRNAs herein. For example, conservative variations of O-tRNA include those molecules that function like the particular O-tRNAs, e.g., as in the sequence listing and examples herein and that maintain the tRNA L-shaped structure by virtue of appropriate self-complementarity, but that do not have a sequence identical to those, e.g., in the sequence listing, figures or examples herein (and, desirably, other than wild type tRNA molecules). See also, the section herein entitled “Nucleic acids and Polypeptides Sequence and Variants.”

The composition comprising an O-tRNA can further include an orthogonal aminoacyl-tRNA synthetase (O-RS), where the O-RS preferentially aminoacylates the O-tRNA with an unnatural amino acid such as OMe-L-tyrosine, α-aminocaprylic acid, or a photoregulated amino acid such as o-nitrobenzyl cysteine or azobenzyl-Phe. In certain embodiments, a composition including an O-tRNA can further include a translation system (e.g., in vitro or in vivo). A nucleic acid that comprises a polynucleotide that encodes a polypeptide of interest, where the polynucleotide comprises a selector codon that is recognized by the O-tRNA, or a combination of one or more of these can also be present in a cell. See also, the section herein entitled “Orthogonal aminoacyl-tRNA synthetases.”

Methods of producing an orthogonal tRNA (O-tRNA) are also a feature of the invention. An O-tRNA produced by the methods is also a feature of the invention. In certain embodiments of the invention, the O-tRNAs can be produced by generating a library of mutants. The library of mutant tRNAs can be generated using various mutagenesis techniques known in the art. For example, the mutant tRNAs can be generated by site-specific mutations, random point mutations, homologous recombination, DNA shuffling or other recursive mutagenesis methods, chimeric construction or any combination thereof.

Additional mutations can be introduced at a specific position(s), e.g., at a nonconservative position(s), or at a conservative position, at a randomized position(s), or a combination of both in a desired loop or region of a tRNA, e.g., an anticodon loop, the acceptor stem, D arm or loop, variable loop, TPC arm or loop, other regions of the tRNA molecule, or a combination thereof. Typically, mutations in a tRNA include mutating the anticodon loop of each member of the library of mutant tRNAs to allow recognition of a selector codon. The method can further include adding an additional sequence (CCA) to a terminus of the O-tRNA. Typically, an O-tRNA possesses an improvement of orthogonality for a desired organism compared to the starting material, e.g., the plurality of tRNA sequences, while preserving its affinity towards a desired RS, etc.

The methods optionally include analyzing the similarity (and/or inferred homology) of sequences of tRNAs and/or aminoacyl-tRNA synthetases to determine potential candidates for an O-tRNA, O-RS and/or pairs thereof, that appear to be orthogonal for a specific organism. Computer programs known in the art and described herein can be used for the analysis, e.g., BLAST and pileup programs can be used. In one example, to choose potential orthogonal translational components for use in E. coli, a prokaryotic organism, a synthetase and/or a tRNA is chosen that does not display close sequence similarity to prokaryotic organisms. Of course, it will be appreciated that in other contexts similar examples as to those below can comprise, e.g., a prokaryote (e.g., E. coli) and a eukaryote (e.g., S. cerevisiae) or a prokaryote (e.g., E. coli) and an Archaea (e.g., M. jannaschii).

Typically, an O-tRNA is obtained by subjecting to, e.g., negative selection, a population of cells of a first species, where the cells comprise a member of the plurality of potential O-tRNAs. The negative selection eliminates cells that comprise a member of the library of potential O-tRNAs that is aminoacylated by an aminoacyl-tRNA synthetase (RS) that is endogenous to the cell. This provides a pool of tRNAs that are orthogonal to the cell of the first species.

In certain embodiments, in the negative selection, a selector codon(s) is introduced into a polynucleotide that encodes a negative selection marker, e.g., an enzyme that confers antibiotic resistance, e.g., β-lactamase, an enzyme that confers a detectable product, e.g., β-galactosidase, chloramphenicol acetyltransferase (CAT), e.g., a toxic product, such as barnase, at a nonessential position (e.g., still producing a functional barnase), etc. Screening/selection is optionally done by growing the population of cells in the presence of a selective agent (e.g., an antibiotic, such as ampicillin). In one embodiment, the concentration of the selection agent is varied.

For example, to measure the activity of suppressor tRNAs, a selection system is used that is based on the in vivo suppression of selector codon, e.g., nonsense or frameshift mutations introduced into a polynucleotide that encodes a negative selection marker, e.g., a gene for β-lactamase (bla). For example, polynucleotide variants, e.g., bla variants, with a selector codon at a certain position (e.g., A184), are constructed. Cells, e.g., bacteria, are transformed with these polynucleotides. In the case of an orthogonal tRNA, which cannot be efficiently charged by endogenous E. coli synthetases, antibiotic resistance, e.g., ampicillin resistance, should be about or less than that for a bacteria transformed with no plasmid. If the tRNA is not orthogonal, or if a heterologous synthetase capable of charging the tRNA is co-expressed in the system, a higher level of antibiotic, e.g., ampicillin, resistance is be observed. Cells, e.g., bacteria, are chosen that are unable to grow on LB agar plates with antibiotic concentrations about equal to cells transformed with no plasmids.

In the case of a toxic product (e.g., ribonuclease or barnase), when a member of the plurality of potential tRNAs is aminoacylated by endogenous host, e.g., Escherichia coli synthetases (i.e., it is not orthogonal to the host, e.g., Escherichia coli synthetases), the selector codon is suppressed and the toxic polynucleotide product produced leads to cell death. Cells harboring orthogonal tRNAs or non-functional tRNAs survive.

In one embodiment, the pool of tRNAs that are orthogonal to a desired organism are then subjected to a positive selection in which a selector codon is placed in a positive selection marker, e.g., encoded by a drug resistance gene, such a β-lactamase gene. The positive selection is performed on a cell comprising a polynucleotide encoding or comprising a member of the pool of tRNAs that are orthogonal to the cell, a polynucleotide encoding a positive selection marker, and a polynucleotide encoding a cognate RS. In certain embodiments, the second population of cells comprises cells that were not eliminated by the negative selection. The polynucleotides are expressed in the cell and the cell is grown in the presence of a selection agent, e.g., ampicillin. tRNAs are then selected for their ability to be aminoacylated by the coexpressed cognate synthetase and to insert an amino acid in response to this selector codon. Typically, these cells show an enhancement in suppression efficiency compared to cells harboring non-functional tRNA(s), or tRNAs that cannot efficiently be recognized by the synthetase of interest. The cell harboring the non-functional tRNAs or tRNAs that are not efficiently recognized by the synthetase of interest, are sensitive to the antibiotic. Therefore, tRNAs that: (i) are not substrates for endogenous host, e.g., Escherichia coli, synthetases; (ii) can be aminoacylated by the synthetase of interest; and (iii) are functional in translation, survive both selections.

Accordingly, the same marker can be either a positive or negative marker, depending on the context in which it is screened. That is, the marker is a positive marker if it is screened for, but a negative marker if screened against.

The stringency of the selection, e.g., the positive selection, the negative selection or both the positive and negative selection, in the above described-methods, optionally includes varying the selection stringency. For example, because barnase is an extremely toxic protein, the stringency of the negative selection can be controlled by introducing different numbers of selector codons into the barnase gene and/or by using an inducible promoter. In another example, the concentration of the selection or screening agent is varied (e.g., ampicillin concentration). In one aspect of the invention, the stringency is varied because the desired activity can be low during early rounds. Thus, less stringent selection criteria are applied in early rounds and more stringent criteria are applied in later rounds of selection. In certain embodiments, the negative selection, the positive selection or both the negative and positive selection can be repeated multiple times. Multiple different negative selection markers, positive selection markers or both negative and positive selection markers can be used. In certain embodiments, the positive and negative selection marker can be the same.

Other types of selections/screening can be used in the invention for producing orthogonal translational components, e.g., an O-tRNA, an O-RS, and an O-tRNA/O-RS pair that loads an unnatural amino acid such as OMe-L-tyrosine, α-aminocaprylic acid, or a photoregulated amino acid such as o-nitrobenzyl cysteine or azobenzyl-Phe in response to a selector codon. For example, the negative selection marker, the positive selection marker or both the positive and negative selection markers can include a marker that fluoresces or catalyzes a luminescent reaction in the presence of a suitable reactant. In another embodiment, a product of the marker is detected by fluorescence-activated cell sorting (FACS) or by luminescence. Optionally, the marker includes an affinity based screening marker. See also, Francisco, J. A., et al., 1993, Proc. Natl. Acad. Sci. USA 90:10444-8.

Additional methods for producing a recombinant orthogonal tRNA can be found, e.g., in International patent applications WO 2002/086075, entitled “Methods and compositions for the production of orthogonal tRNA-aminoacyltRNA synthetase pairs”; WO 2004/094593, entitled “EXPANDING THE EUKARYOTIC GENETIC CODE”; and, International Application Number PCT/US2004/02187, filed Jul. 7, 2004. See also Forster et al., 2003, Proc. Natl. Acad. Sci. USA 100(11):6353-6357; and, Feng et al., 2003, Proc. Natl. Acad. Sci. USA 100(10): 5676-5681.

Orthogonal Aminoacyl-tRNA Synthetase (O-RS)

An O-RS of the invention preferentially aminoacylates an O-tRNA, e.g., a leucyl O-tRNA or tyrosyl O-tRNA as in the examples herein, with an unnatural amino acid such as OMe-L-tyrosine, α-aminocaprylic acid, or a photoregulated amino acid such as o-nitrobenzyl cysteine or azobenzyl-Phe in vitro or in vivo. An O-RS of the invention (e.g., as in the examples and sequence listing herein) can be provided to the translation system, e.g., a cell, by a polypeptide that includes an O-RS and/or by a polynucleotide that encodes an O-RS or a portion thereof. For example, an example O-RS comprises an amino acid sequence as set forth herein, e.g., in the Sequence Listing and in the examples herein, or a conservative variation thereof. In another example, an O-RS, or a portion thereof, is encoded by a polynucleotide sequence that encodes an amino acid comprising sequence herein (e.g., in the Sequence Listing) or in the examples herein, or a complementary polynucleotide sequence thereof. See, e.g., the tables and examples herein for sequences of exemplary O-RS molecules. See also, the section entitled “Nucleic Acid and Polypeptide Sequence and Variants” herein.

Methods for identifying an orthogonal aminoacyl-tRNA synthetase (O-RS), e.g., an O-RS, for use with an O-tRNA, are also a feature of the invention. For example, a method includes subjecting to selection, e.g., positive selection, a population of cells of a first species, where the cells individually comprise: 1) a member of a plurality of aminoacyl-tRNA synthetases (RSs), (e.g., the plurality of RSs can include mutant RSs, RSs derived from a species other than the first species or both mutant RSs and RSs derived from a species other than the first species); 2) the orthogonal tRNA (O-tRNA) (e.g., from one or more species); and 3) a polynucleotide that encodes an (e.g., positive) selection marker and comprises at least one selector codon. Cells are selected or screened for those that show an enhancement in suppression efficiency compared to cells lacking or with a reduced amount of the member of the plurality of RSs. Suppression efficiency can be measured by techniques known in the art and as described herein. Cells having an enhancement in suppression efficiency comprise an active RS that aminoacylates the O-tRNA. A level of aminoacylation (in vitro or in vivo) by the active RS of a first set of tRNAs from the first species is compared to the level of aminoacylation (in vitro or in vivo) by the active RS of a second set of tRNAs from the second species. The level of aminoacylation can be determined by a detectable substance (e.g., a labeled amino acid or unnatural amino acid, e.g., a labeled OMe-L-tyrosine, α-aminocaprylic acid, or photoregulated amino acid such as o-nitrobenzyl cysteine or azobenzyl-Phe). The active RS that more efficiently aminoacylates the second set of tRNAs compared to the first set of tRNAs is typically selected, thereby providing an efficient (optimized) orthogonal aminoacyl-tRNA synthetase for use with the O-tRNA. An O-RS, identified by the method, is also a feature of the invention.

Any of a number of assays can be used to determine aminoacylation. These assays can be performed in vitro or in vivo. For example, in vitro aminoacylation assays are described in, e.g., Hoben and Soll (1985) Methods Enzymol., 113:55-59. Aminoacylation can also be determined by using a reporter along with orthogonal translation components and detecting the reporter in a cell expressing a polynucleotide comprising at least one selector codon that encodes a protein. See also, WO 2002/085923, entitled “IN VIVO INCORPORATION OF UNNATURAL AMINO ACIDS”; WO 2004/094593; International Application Number PCT/US2004/011786, filed Apr. 16, 2004; and International Application Number PCT/US2004/022187, filed Jul. 7, 2004.

Identified O-RS can be further manipulated to alter substrate specificity of the synthetase, so that only a desired unnatural amino acid, e.g., OMe-L-tyrosine, α-aminocaprylic acid, or a photoregulated amino acid such as o-nitrobenzyl cysteine or azobenzyl-Phe, but not any of the common 20 amino acids, are charged to the O-tRNA. Methods to generate an orthogonal aminoacyl tRNA synthetase with a substrate specificity for an unnatural amino acid include mutating the synthetase, e.g., at the active site in the synthetase, at the editing mechanism site in the synthetase, at different sites by combining different domains of synthetases, or the like, and applying a selection process. A strategy is used, which is based on the combination of a positive selection followed by a negative selection. In the positive selection, suppression of the selector codon introduced at a nonessential position(s) of a positive marker allows cells to survive under positive selection pressure. In the presence of both natural and unnatural amino acids, survivors thus encode active synthetases charging the orthogonal suppressor tRNA with either a natural or unnatural amino acid. In the negative selection, suppression of a selector codon introduced at a nonessential position(s) of a negative marker removes synthetases with natural amino acid specificities. Survivors of the negative and positive selection encode synthetases that aminoacylate (charge) the orthogonal suppressor tRNA with unnatural amino acids only. These synthetases can then be subjected to further mutagenesis, e.g., DNA shuffling or other recursive mutagenesis methods.

A library of mutant O-RSs can be generated using various mutagenesis techniques known in the art. For example, the mutant RSs can be generated by site-specific mutations, random point mutations, homologous recombination, DNA shuffling or other recursive mutagenesis methods, chimeric construction or any combination thereof. For example, a library of mutant RSs can be produced from two or more other, e.g., smaller, less diverse “sub-libraries.” Chimeric libraries of RSs are also included in the invention. It should be noted that libraries of tRNA synthetases from various organism (e.g., microorganisms such as eubacteria or archaeabacteria) such as libraries that comprise natural diversity (see, e.g., U.S. Pat. No. 6,238,884 to Short et al; U.S. Pat. No. 5,756,316 to Schallenberger et al; U.S. Pat. No. 5,783,431 to Petersen et al; U.S. Pat. No. 5,824,485 to Thompson et al; U.S. Pat. No. 5,958,672 to Short et al), are optionally constructed and screened for orthogonal pairs.

Once the synthetases are subject to the positive and negative selection/screening strategy, these synthetases can then be subjected to further mutagenesis. For example, a nucleic acid that encodes the O-RS can be isolated; a set of polynucleotides that encode mutated O-RSs (e.g., by random mutagenesis, site-specific mutagenesis, recombination or any combination thereof) can be generated from the nucleic acid; and, these individual steps or a combination of these steps can be repeated until a mutated O-RS is obtained that preferentially aminoacylates the O-tRNA with the unnatural amino acid, e.g., OMe-L-tyrosine, α-aminocaprylic acid, or a photoregulated amino acid such as o-nitrobenzyl cysteine or azobenzyl-Phe. In one aspect of the invention, the steps are performed multiple times, e.g., at least two times.

Additional levels of selection/screening stringency can also be used in the methods of the invention, for producing O-tRNA, O-RS, or pairs thereof. The selection or screening stringency can be varied on one or both steps of the method to produce an O-RS. This could include, e.g., varying the amount of selection/screening agent that is used, etc. Additional rounds of positive and/or negative selections can also be performed. Selecting or screening can also comprise one or more of a change in amino acid permeability, a change in translation efficiency, a change in translational fidelity, etc. Typically, the one or more change is based upon a mutation in one or more gene in an organism in which an orthogonal tRNA-tRNA synthetase pair is used to produce protein.

Additional general details for producing O-RS, and altering the substrate specificity of the synthetase can be found in WO 2002/086075 entitled “Methods and compositions for the production of orthogonal tRNA-aminoacyltRNA synthetase pairs”; and International Application Number PCT/US2004/011786, filed Apr. 16, 2004.

### Source and Host Organisms

The orthogonal translational components (O-tRNA and O-RS) of the invention can be derived from any organisms (or combination or organisms) for use in a host translation system from any other species, with the caveat that the O-tRNA/O-RS components and the host system work in an orthogonal manner. It is not a requirement that the O-tRNA and the O-RS be derived from the same organisms. In various aspects, the orthogonal components can be derived from Archaea genes (i.e., from an archaeabacteria) for use in a eubacterial host system or from eubacterial genes for use in a eukaryotic host system.

For example, the orthogonal O-tRNA can be derived from a prokaryotic (non-eukaryotic) organism (or a combination of organisms), e.g., an archaebacterium, such as Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, Methanococcus maripaludis, Methanopyrus kandleri, Methanosarcina mazei (Mm), Pyrobaculum aerophilum, Pyrococcus abyssi, Sulfolobus solfataricus (Ss), Sulfolobus tokodaii, Themmoplasma acidophilum, Thermoplasma volcanium, or the like, or a eubacterium, such as Escherichia coli, Thermus thermophilus, Bacillus stearothenmphilus, or the like, while the orthogonal O-RS can be derived from an organism (or a combination of organisms), e.g., an archaebacterium, such as Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, Methanococcus maripaludis, Methanopyrus kandleri, Methanosarcina mazei, Pyrobaculum aerophilum, Pyrococcus abyssi, Sulfolobus solfataricus, Sulfolobus tokodaii, Thernoplasma acidophilum, Thermoplasma volcanium, or the like, or a eubacterium, such as Escherichia coli, Thermus thermophilus, Bacillus stearothermphilus, or the like. In some embodiments, eukaryotic sources, e.g., plants, algae, protists, fungi, yeasts (e.g., S. cerevisiae), animals (e.g., mammals, insects, arthropods, etc.), or the like, can also be used as sources of O-tRNAs and O-RSs.

The individual components of an O-tRNA/O-RS pair can be derived from the same organism or different organisms. In certain embodiments, the O-tRNA/O-RS pair is from the same organism. Alternatively, the O-tRNA and the O-RS of the O-tRNA/O-RS pair are from different organisms. In one example embodiment, the leucyl synthetase/tRNA pair of E. coli is used as an orthogonal pair, e.g., in a yeast-based translation system. As described herein, this pair can be modified to recognize an amber selector codon and can be modified to charge the O-tRNA with an unnatural amino acid such as O-Me-L-tyrosine, α-aminocaprylic acid, or a photoregulated amino acid such as o-nitrobenzyl cysteine. This orthogonal pair (or modified forms thereof) can also be combined with previously described orthogonal pairs, e.g., those derived from Methanococcus jannaschii, e.g., that are modified to recognize other selector codons. This provides for production of proteins that comprise two different unnatural amino acids in a translation system of interest by including a coding nucleic acid for such proteins that include two or more selector codons that are each recognized by an O-tRNA/O-RS pair. Other embodiments can also present pairs, e.g., Example 2 which comprises orthogonal pairs from M. jannaschii (an Archaea) used as an orthogonal pair in a eubacterial (E. coli) translation system. See below.

The O-tRNA, O-RS or O-tRNA/O-RS pair can be selected or screened in vivo or in vitro and/or used in a cell, e.g., a non-eukaryotic, or prokaryotic, cells, or eukaryotic cells, to produce a polypeptide with an OMe-L-tyrosine, α-aminocaprylic acid, or photoregulated amino acid such as o-nitrobenzyl cysteine or azobenzyl-Phe, or other unnatural amino acid of interest. A non-eukaryotic cell can be from any of a variety of sources, e.g., a eubacterium, such as Escherichia coli, Thermus thermophilus, Bacillus stearothermphilus, or the like, or an archaebacterium, such as Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, Methanococcus maripaludis, Methanopyrus kandleri, Methanosarcina mazei (Mm), Pyrobaculum aerophiluin, Pyrococcus abyssi, Sulfolobus solfataricus (Ss), Sulfolobus tokodaii, Thermoplasma acidophilum, Thermoplasma volcanium, or the like. A eukaryotic cell can be from any of a variety of sources, e.g., a plant (e.g., complex plant such as monocots, or dicots), an algae, a protist, a fungus, a yeast (e.g., Saccharomyces cerevisiae), an animal (e.g., a mammal, an insect, an arthropod, etc.), or the like. Compositions of cells with translational components of the invention are also a feature of the invention.

See also, International Application Number PCT/US2004/011786, filed Apr. 16, 2004, for screening O-tRNA and/or O-RS in one species for use in another species.

### Selector Codons

Selector codons of the invention expand the genetic codon framework of the protein biosynthetic machinery. For example, a selector codon includes, e.g., a unique three base codon, a nonsense codon, such as a stop codon, e.g., an amber codon (UAG), or an opal codon (UGA), an unnatural codon, an at least a four base codon (e.g., AGGA), a rare codon, or the like. A number of selector codons can be introduced into a desired gene, e.g., one or more, two or more, more than three, etc. By using different selector codons, multiple orthogonal tRNA/synthetase pairs can be used that allow the simultaneous site-specific incorporation of multiple different unnatural amino acids, e.g., OMe-L-tyrosine, α-aminocaprylic acid, or a photoregulated amino acid such as o-nitrobenzyl cysteine or azobenzyl-Phe, using these different selector codons.

In one embodiment, the methods involve the use of a selector codon that is a stop codon for the incorporation of OMe-L-tyrosine, α-aminocaprylic acid, or a photoregulated amino acid such as o-nitrobenzyl cysteine or azobenzyl-Phe in vivo in a cell. For example, as in Example 1 herein, an O-tRNA is produced that recognizes an amber codon (an amber nonsense codon in yeast) and is aminoacylated by an O-RS with OMe-L-tyrosine, α-aminocaprylic acid, or a photoregulated amino acid such as o-nitrobenzyl cysteine. This O-tRNA is not recognized by the translation system's endogenous aminoacyl-tRNA synthetases. Conventional site-directed mutagenesis can be used to introduce the selector codon at the site of interest in a target polynucleotide encoding a polypeptide of interest. See also, e.g., Sayers, J. R., et al. (1988), “5′,3′ Exonuclease in phosphorothioate-based oligonucleotide-directed mutagenesis” Nucleic Acids Res., 791-802. When the O-RS, O-tRNA and the nucleic acid that encodes a polypeptide of interest are combined, e.g., in vivo, the OMe-L-tyrosine, α-aminocaprylic acid, or photoregulated amino acid such as o-nitrobenzyl cysteine or azobenzyl-Phe is incorporated in response to the selector codon to give a polypeptide containing the OMe-L-tyrosine, α-aminocaprylic acid, or a photoregulated amino acid such as o-nitrobenzyl cysteine or azobenzyl-Phe at the specified position.

The incorporation of unnatural amino acids such as OMe-L-tyrosine, α-aminocaprylic acid, or a photoregulated amino acid such as o-nitrobenzyl cysteine or azobenzyl-Phe in vivo, can be done without significant perturbation of the host cell. For example, in prokaryotic cells, such as Escherichia coli, because the suppression efficiency of a stop selector codon, the UAG codon, depends upon the competition between the O-tRNA, e.g., the amber suppressor tRNA, and release factor 1 (RF1) (which binds to the UAG codon and initiates release of the growing peptide from the ribosome), the suppression efficiency can be modulated by, e.g., either increasing the expression level of O-tRNA, e.g., the suppressor tRNA, or using an RF1 deficient strain. In eukaryotic cells, because the suppression efficiency for a UAG codon depends upon the competition between the O-tRNA, e.g., the amber suppressor tRNA, and a eukaryotic release factor (e.g., eRF) (which binds to a stop codon and initiates release of the growing peptide from the ribosome), the suppression efficiency can be modulated by, e.g., increasing the expression level of O-tRNA, e.g., the suppressor tRNA. In addition, additional compounds can also be present that modulate release factor action, e.g., reducing agents such as dithiothreitol (DTT).

Unnatural amino acids, including, e.g., a OMe-L-tyrosine, α-aminocaprylic acid, or a photoregulated amino acid such as o-nitrobenzyl cysteine or azobenzyl-Phe can also be encoded with rare codons. For example, when the arginine concentration in an in vitro protein synthesis reaction is reduced, the rare arginine codon, AGG, has proven to be efficient for insertion of Ala by a synthetic tRNA acylated with alanine. See, e.g., Ma et al., Biochemistry, 32:7939 (1993). In this case, the synthetic tRNA competes with the naturally occurring tRNA_{Arg}, which exists as a minor species in Escherichia coli. In addition, some organisms do not use all triplet codons. An unassigned codon AGA in Micrococcus luteus has been utilized for insertion of amino acids in an in vitro transcription/translation extract. See, e.g., Kowal and Oliver, Nucl. Acid. Res., 25:4685 (1997). Components of the invention can be generated to use these rare codons in vivo.

Selector codons can also comprise extended codons, e.g., four or more base codons, such as, four, five, six or more base codons. Examples of four base codons include, e.g., AGGA, CUAG, UAGA, CCCU, and the like. Examples of five base codons include, e.g., AGGAC, CCCCU, CCCUC, CUAGA, CUACU, UAGGC, and the like. Methods of the invention include using extended codons based on frameshift suppression. Four or more base codons can insert, e.g., one or multiple unnatural amino acids, such as OMe-L-tyrosine, α-aminocaprylic acid, or a photoregulated amino acid such as o-nitrobenzyl cysteine or azobenzyl-Phe into the same protein. In other embodiments, the anticodon loops can decode, e.g., at least a four-base codon, at least a five-base codon, or at least a six-base codon or more. Since there are 256 possible four-base codons, multiple unnatural amino acids can be encoded in the same cell using a four or more base codon. See also, Anderson et al., (2002) “Exploring the Limits of Codon and Anticodon Size,” Chemistry and Biology, 9:237-244; and, Magliery, (2001) “Expanding the Genetic Code: Selection of Efficient Suppressors of Four-base Codons and Identification of “Shifty” Four-base Codons with a Library Approach in Escherichia coli,” J. Mol. Biol. 307: 755-769.

For example, four-base codons have been used to incorporate unnatural amino acids into proteins using in vitro biosynthetic methods. See, e.g., Ma et al., (1993) Biochemistry, 32:7939; and Hohsaka et al., (1999) J. Am. Chem. Soc., 121:34. CGGG and AGGU were used to simultaneously incorporate 2-naphthylalanine and an NBD derivative of lysine into streptavidin in vitro with two chemically acylated frameshift suppressor tRNAs. See, e.g., Hohsaka et al., (1999) J. Am. Chem. Soc., 121:12194. In an in vivo study, Moore et al. examined the ability of tRNA^{Leu} derivatives with NCUA anticodons to suppress UAGN codons (N can be U, A, G, or C), and found that the quadruplet UAGA can be decoded by a tRNA^{Leu} with a UCUA anticodon with an efficiency of 13 to 26% with little decoding in the 0 or −1 frame. See Moore et al., (2000) J. Mol. Biol., 298:195. In one embodiment, extended codons based on rare codons or nonsense codons can be used in invention, which can reduce missense readthrough and frameshift suppression at other unwanted sites.

For a given system, a selector codon can also include one of the natural three base codons, where the endogenous system does not use (or rarely uses) the natural base codon. For example, this includes a system that is lacking a tRNA that recognizes the natural three base codon, and/or a system where the three base codon is a rare codon.

Selector codons optionally include unnatural base pairs. These unnatural base pairs further expand the existing genetic alphabet. One extra base pair increases the number of triplet codons from 64 to 125. Properties of third base pairs include stable and selective base pairing, efficient enzymatic incorporation into DNA with high fidelity by a polymerase, and the efficient continued primer extension after synthesis of the nascent unnatural base pair. Descriptions of unnatural base pairs which can be adapted for methods and compositions include, e.g., Hirao, et al., (2002) “An unnatural base pair for incorporating amino acid analogues into protein,” Nature Biotechnology, 20:177-182. See also Wu, Y., et al., (2002) J. Am. Chem. Soc. 124:14626-14630. Other relevant publications are listed elsewhere herein.

For in vivo usage, the unnatural nucleoside is membrane permeable and is phosphorylated to form the corresponding triphosphate. In addition, the increased genetic information is stable and not destroyed by cellular enzymes. Previous efforts by Benner and others took advantage of hydrogen bonding patterns that are different from those in canonical Watson-Crick pairs, the most noteworthy example of which is the iso-C:iso-G pair. See, e.g., Switzer et al., (1989) J. Am. Chem. Soc., 111:8322; Piccirilli et al., (1990) Nature, 343:33; and Kool, (2000) Curr. Opin. Chem. Biol., 4:602. These bases in general, mispair to some degree with natural bases and cannot be enzymatically replicated. Kool and co-workers demonstrated that hydrophobic packing interactions between bases can replace hydrogen bonding to drive the formation of base pair. See Kool, (2000) Curr. Opin. Chem. Biol., 4:602; and Guckian and Kool, (1998) Angew. Chem. Int. Ed. Engl., 36, 2825. In an effort to develop an unnatural base pair satisfying all the above requirements, Schultz, Romesberg and co-workers have systematically synthesized and studied a series of unnatural hydrophobic bases. A PICS:PICS self-pair is found to be more stable than natural base pairs, and can be efficiently incorporated into DNA by Klenow fragment of Escherichia coli DNA polymerase I (KF). See, e.g., McMinn et al., (1999) J. Am. Chem. Soc., 121:11586; and Ogawa et al., (2000) J. Am. Chem. Soc., 122:3274. A 3MN:3MN self-pair can be synthesized by KF with efficiency and selectivity sufficient for biological function. See, e.g., Ogawa et al., (2000) J. Am. Chem. Soc., 122:8803. However, both bases act as a chain terminator for further replication. A mutant DNA polymerase has been recently evolved that can be used to replicate the PICS self pair. In addition, a 7AI self pair can be replicated. See, e.g., Tae et al., (2001) J. Am. Chem. Soc., 123:7439. A novel metallobase pair, Dipic:Py, has also been developed, which forms a stable pair upon binding Cu(II). See Meggers et al., (2000) J. Am. Chem. Soc., 122:10714. Because extended codons and unnatural codons are intrinsically orthogonal to natural codons, the methods of the invention can take advantage of this property to generate orthogonal tRNAs for them.

A translational bypassing system can also be used to incorporate an OMe-L-tyrosine, α-aminocaprylic acid, or a photoregulated amino acid such as o-nitrobenzyl cysteine or azobenzyl-Phe, or other unnatural amino acid into a desired polypeptide. In a translational bypassing system, a large sequence is inserted into a gene but is not translated into protein. The sequence contains a structure that serves as a cue to induce the ribosome to hop over the sequence and resume translation downstream of the insertion.

### Unnatural Amino Acids

As used herein, an unnatural amino acid refers to any amino acid, modified amino acid, or amino acid analogue other than selenocysteine and/or pyrolysine and the following twenty genetically encoded alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine. The generic structure of an alpha-amino acid is illustrated by Formula I:

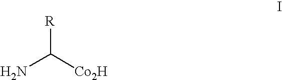

An unnatural amino acid is typically any structure having Formula I wherein the R group is any substituent other than one used in the twenty natural amino acids. See, e.g., Biochemistry by L. Stryer, 3^{rd} ed. 1988, Freeman and Company, New York, for structures of the twenty natural amino acids. Note that, the unnatural amino acids of the invention can be naturally occurring compounds other than the twenty alpha-amino acids above (or, of course, artificially produced synthetic compounds).

Because the unnatural amino acids of the invention typically differ from the natural amino acids in side chain, the unnatural amino acids form amide bonds with other amino acids, e.g., natural or unnatural, in the same manner in which they are formed in naturally occurring proteins. However, the unnatural amino acids have side chain groups that distinguish them from the natural amino acids.

Of particular interest herein are unnatural amino acids such as OMe-L-tyrosine, α-aminocaprylic acid, or a photoregulated amino acid such as o-nitrobenzyl cysteine or azobenzyl-Phe.

In other unnatural amino acids, for example, R in Formula I optionally comprises an alkyl-, aryl-, acyl-, keto-, azido-, hydroxyl-, hydrazine, cyano-, halo-, hydrazide, alkenyl, alkynyl, ether, thiol, seleno-, sulfonyl-, borate, boronate, phospho, phosphono, phosphine, heterocyclic, enone, imine, aldehyde, ester, thioacid, hydroxylamine, amine, and the like, or any combination thereof. Other unnatural amino acids of interest include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, biotin or biotin-analogue containing amino acids, keto containing amino acids, glycosylated amino acids, amino acids comprising polyethylene glycol or polyether, heavy atom substituted amino acids, chemically cleavable or photocleavable amino acids, amino acids with an elongated side chain as compared to natural amino acids (e.g., polyethers or long chain hydrocarbons, e.g., greater than about 5, greater than about 10 carbons, etc.), carbon-linked sugar-containing amino acids, redox-active amino acids, amino thioacid containing amino acids, and amino acids containing one or more toxic moiety. In some embodiments, the unnatural amino acids have a photoactivatable cross-linker. In one embodiment, the unnatural amino acids have a saccharide moiety attached to the amino acid side chain and/or other carbohydrate modification.

In addition to unnatural amino acids that contain novel side chains, unnatural amino acids also optionally comprise modified backbone structures, e.g., as illustrated by the structures of Formula II and III:

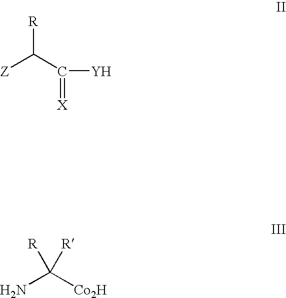

werein Z typically comprises OH, NIH2, SH, NH—R′, or S—R′; X and Y, which can be the same or different, typically comprise S or O, and R and R′, which are optionally the same or different, are typically selected from the same list of constituents for the R group described above for the unnatural amino acids having Formula I as well as hydrogen. For example, unnatural amino acids of the invention optionally comprise substitutions in the amino or carboxyl group as illustrated by Formulas II and III. Unnatural amino acids of this type include, but are not limited to, α-hydroxy acids, α-thioacids α-aminothiocarboxylates, e.g., with side chains corresponding to the common twenty natural amino acids or unnatural side chains. In addition, substitutions at the α-carbon optionally include L, D, or α-α-disubstituted amino acids such as D-glutamate, D-alanine, D-methyl-O-tyrosine, aminobutyric acid, and the like. Other structural alternatives include cyclic amino acids, such as proline analogues as well as 3, 4, 6, 7, 8, and 9 membered ring proline analogues, β and γ amino acids such as substituted β-alanine and γ-amino butyric acid. Additional unnatural amino acid structures of the invention include homo-beta-type structures, e.g., where there is, e.g., a methylene or amino group sandwiched adjacent to the alpha carbon, e.g., isomers of homo-beta-tyrosine, alpha-hydrazino-tyrosine.

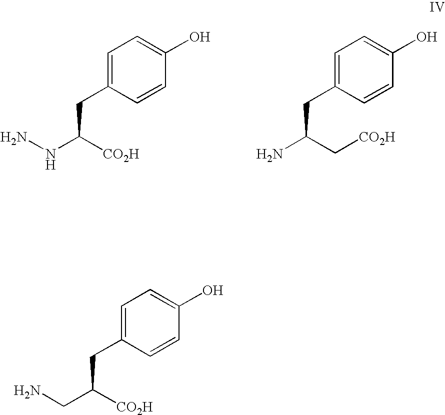

Many unnatural amino acids are based on natural amino acids, such as tyrosine, glutamine, phenylalanine, and the like. For example, tyrosine analogs include para-substituted tyrosines, ortho-substituted tyrosines, and meta substituted tyrosines, wherein the substituted tyrosine comprises an acetyl group, a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, or the like. In addition, multiply substituted aryl rings are also contemplated. Glutamine analogs of the invention include, but are not limited to, α-hydroxy derivatives, γ-substituted derivatives, cyclic derivatives, and amide substituted glutamine derivatives. Example phenylalanine analogs include, but are not limited to, para-substituted phenylalanines, ortho-substituted phenylalanines, and meta-substituted phenylalanines, wherein the substituent comprises a hydroxy group, a methoxy group, a methyl group, an allyl group, an aldehyde or keto group, or the like. Specific examples of unnatural amino acids include, but are not limited to, homoglutamine, a 3,4-dihydroxy-L-phenylalanine, a p-acetyl-L-phenylalanine, a p-propargyloxyphenylalanine, O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like. The structures of a variety of unnatural amino acids are provided in, for example, FIGS. 16, 17, 18, 19, 26, and 29 of WO 2002/085923 entitled “In vivo incorporation of unnatural amino acids” and Published International Application WO 2004/094593, entitled “Expanding the Eukaryotic Genetic Code.”

Chemical Synthesis of Unnatural Amino Acids

Many of the unnatural amino acids provided above are commercially available, e.g., from Sigma (St. Louis, Mo.) or Aldrich (Milwaukee, Wis.). Those that are not commercially available are optionally synthesized as provided in various publications or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional publications describing the synthesis of unnatural amino acids include, e.g., WO 2002/085923 entitled “In vivo incorporation of Unnatural Amino Acids;” Matsoukas et al., (1995) J. Med. Chem., 38, 4660-4669; King, F. E. & Kidd, D. A. A. (1949) “A New Synthesis of Glutamine and of γ-Dipeptides of Glutamic Acid from Phthylated Intermediates” J. Chem. Soc., 3315-3319; Friedman, O. M. & Chatterrji, R. (1959) “Synthesis of Derivatives of Glutamine as Model Substrates for Anti-Tumor Agents” J. Am. Chem. Soc. 81, 3750-3752; Craig, J. C. et al. (1988) “Absolute Configuration of the Enantiomers of 7-Chloro-4 [[4-(diethylamino)-1-methylbutyl]amino]quinoline (Chloroquine)” J. Org. Chem. 53, 1167-1170; Azoulay, M., Vilmont, M. & Frappier, F. (1991) “Glutamine analogues as Potential Antimalarials” Eur. J. Med. Chem. 26, 201-5; Koskinen, A. M. P. & Rapoport, H. (1989) “Synthesis of 4-Substituted Prolines as Conformationally Constrained Amino Acid Analogue” J. Org. Chem. 54, 1859-1866; Christie, B. D. & Rapoport, H. (1985) “Synthesis of Optically Pure Pipecolates from L-Asparagine. Application to the Total Synthesis of (+)-Apovincamine through Amino Acid Decarbonylation and Iminium Ion Cyclization” J. Org. Chem. 1989:1859-1866; Barton et al., (1987) “Synthesis of Novel a-Amino-Acids and Derivatives Using Radical Chemistry: Synthesis of L- and D-a-Amino-Adipic Acids, L-a-aminopimelic Acid and Appropriate Unsaturated Derivatives” Tetrahedron Lett. 43:4297-4308; and, Subasinghe et al., (1992) “Quisqualic acid analogues: synthesis of beta-heterocyclic 2-aminopropanoic acid derivatives and their activity at a novel quisqualate-sensitized site” J. Med. Chem. 35:4602-7. See also International Application Number PCT/US03/41346, entitled “Protein Arrays,” filed on Dec. 22, 2003.

Cellular Uptake of Unnatural Amino Acids

Unnatural amino acid uptake by a cell is one issue that is typically considered when designing and selecting unnatural amino acids, e.g., for incorporation into a protein. For example, the high charge density of α-amino acids suggests that these compounds are unlikely to be cell permeable. Natural amino acids are taken up into the cell via a collection of protein-based transport systems often displaying varying degrees of amino acid specificity. A rapid screen can be done which assesses which unnatural amino acids, if any, are taken up by cells. See, e.g., toxicity assays in, e.g., International Application Number PCT/US03/41346, entitled “Protein Arrays,” filed on Dec. 22, 2003; and Liu, D. R. & Schultz, P. G. (1999) “Progress toward the evolution of an organism with an expanded genetic code” Proc. Natl. Acad. Sci. USA 96:4780-4785. Although uptake is easily analyzed with various assays, an alternative to designing unnatural amino acids that are amenable to cellular uptake pathways is to provide biosynthetic pathways to create amino acids in vivo.

Biosynthesis of Unnatural Amino Acids

Many biosynthetic pathways already exist in cells for the production of amino acids and other compounds. While a biosynthetic method for a particular unnatural amino acid may not exist in nature, e.g., in a cell, the invention provides such methods. For example, biosynthetic pathways for unnatural amino acids are optionally generated in host cell by adding new enzymes or modifying existing host cell pathways. Additional new enzymes are optionally naturally occurring enzymes or artificially evolved enzymes. For example, the biosynthesis of p-aminophenylalanine (as presented in an example in WO 2002/085923, supra) relies on the addition of a combination of known enzymes from other organisms. The genes for these enzymes can be introduced into a cell by transforming the cell with a plasmid comprising the genes. The genes, when expressed in the cell, provide an enzymatic pathway to synthesize the desired compound. Examples of the types of enzymes that are optionally added are found, e.g., in Genbank. Artificially evolved enzymes are also optionally added into a cell in the same manner. In this manner, the cellular machinery and resources of a cell are manipulated to produce unnatural amino acids.

Indeed, any of a variety of methods can be used for producing novel enzymes for use in biosynthetic pathways, or for evolution of existing pathways, for the production of unnatural amino acids, in vitro or in vivo. Many available methods of evolving enzymes and other biosynthetic pathway components can be applied to the present invention to produce unnatural amino acids (or, indeed, to evolve synthetases to have new substrate specificities or other activities of interest). For example, DNA shuffling is optionally used to develop novel enzymes and/or pathways of such enzymes for the production of unnatural amino acids (or production of new synthetases), in vitro or in vivo. See, e.g., Stemmer (1994) “Rapid evolution of a protein in vitro by DNA shuffling” Nature 370(4):389-391; and, Stemmer, (1994) “DNA shuffling by random fragmentation and reassembly: In vitro recombination for molecular evolution” Proc. Natl. Acad. Sci. USA, 91:10747-10751. A related approach shuffles families of related (e.g., homologous) genes to quickly evolve enzymes with desired characteristics. An example of such “family gene shuffling” methods is found in Crameri et al. (1998) “DNA shuffling of a family of genes from diverse species accelerates directed evolution,” Nature, 391(6664): 288-291. New enzymes (whether biosynthetic pathway components or synthetases) can also be generated using a DNA recombination procedure known as “incremental truncation for the creation of hybrid enzymes” (“ITCHY”), e.g., as described in Ostermeier et al. (1999) “A combinatorial approach to hybrid enzymes independent of DNA homology,” Nature Biotech. 17:1205. This approach can also be used to generate a library of enzyme or other pathway variants which can serve as substrates for one or more in vitro or in vivo recombination methods. See, also, Ostermeier et al. (1999) “Combinatorial Protein Engineering by Incremental Truncation,” Proc. Natl. Acad. Sci. USA, 96: 3562-67, and Ostermeier et al. (1999) “Incremental Truncation as a Strategy in the Engineering of Novel Biocatalysts,” Biological and Medicinal Chemistry, 7: 2139-44. Another approach uses exponential ensemble mutagenesis to produce libraries of enzyme or other pathway variants that are, e.g., selected for an ability to catalyze a biosynthetic reaction relevant to producing an unnatural amino acid (or a new synthetase). In this approach, small groups of residues in a sequence of interest are randomized in parallel to identify, at each altered position, amino acids which lead to functional proteins. Examples of such procedures, which can be adapted to the present invention to produce new enzymes for the production of unnatural amino acids (or new synthetases) are found in Delegrave & Youvan (1993) Biotechnology Research 11:1548-1552. In yet another approach, random or semi-random mutagenesis using doped or degenerate oligonucleotides for enzyme and/or pathway component engineering can be used, e.g., by using the general mutagenesis methods of e.g., Arkin and Youvan (1992) “Optimizing nucleotide mixtures to encode specific subsets of amino acids for semi-random mutagenesis” Biotechnology 10:297-300; or Reidhaar-Olson et al. (1991) “Random mutagenesis of protein sequences using oligonucleotide cassettes” Methods Enzymol. 208:564-86. Yet another approach, often termed a “non-stochastic” mutagenesis, which uses polynucleotide reassembly and site-saturation mutagenesis can be used to produce enzymes and/or pathway components, which can then be screened for an ability to perform one or more synthetase or biosynthetic pathway function (e.g., for the production of unnatural amino acids in vivo). See, e.g., Short “Non-Stochastic Generation of Genetic Vaccines and Enzymes,” WO 00/46344.

An alternative to such mutational methods involves recombining entire genomes of organisms and selecting resulting progeny for particular pathway functions (often referred to as “whole genome shuffling”). This approach can be applied to the present invention, e.g., by genomic recombination and selection of an organism (e.g., an E. coli or other cell) for an ability to produce an unnatural amino acid (or intermediate thereof). For example, methods taught in the following publications can be applied to pathway design for the evolution of existing and/or new pathways in cells to produce unnatural amino acids in vivo: Patnaik et al. (2002) “Genome shuffling of lactobacillus for improved acid tolerance,” Nature Biotechnology, 20(7): 707-712; and Zhang et al. (2002) “Genome shuffling leads to rapid phenotypic improvement in bacteria,” Nature, February 7, 415(6872): 644-646.

Other techniques for organism and metabolic pathway engineering, e.g., for the production of desired compounds are also available and can also be applied to the production of unnatural amino acids. Examples of publications teaching useful pathway engineering approaches include: Nakamura and White (2003) “Metabolic engineering for the microbial production of 1,3 propanediol” Curr. Opin. Biotechnol. 14(5):454-9; Berry et al. (2002) “Application of Metabolic Engineering to improve both the production and use of Biotech Indigo” J. Industrial Microbiology and Biotechnology 28:127-133; Banta et al. (2002) “Optimizing an artificial metabolic pathway: Engineering the cofactor specificity of Corynebacterium 2,5-diketo-D-gluconic acid reductase for use in vitamin C biosynthesis” Biochemistry, 41(20), 6226-36; Selivonova et al. (2001) “Rapid Evolution of Novel Traits in Microorganisms” Applied and Environmental Microbiology, 67:3645, and many others.

Regardless of the method used, typically, the unnatural amino acid produced with an engineered biosynthetic pathway of the invention is produced in a concentration sufficient for efficient protein biosynthesis, e.g., a natural cellular amount, but not to such a degree as to significantly affect the concentration of other cellular amino acids or to exhaust cellular resources. Typical concentrations produced in vivo in this manner are about 10 mM to about 0.05 mM. Once a cell is engineered to produce enzymes desired for a specific pathway and an unnatural amino acid is generated, in vivo selections are optionally used to further optimize the production of the unnatural amino acid for both ribosomal protein synthesis and cell growth.

It will be appreciated that the various unnatural amino acids can also comprise photoregulated amino acids. See below. Additionally, in optional embodiments herein, the amino acids involved (e.g., those added to peptide chains by the tRNA/O-RS pairs of the invention) can comprise an azobenzyl-Phe side chain, an azobenzyl-Phe, or a diphenyldiazene. See below.

Photoregulated Unnatural Amino Acids

Photoregulated amino acids (e.g., photochromic, photocleavable, photoisomerizable, etc.) can be used to spatially and temporally control a variety of biological process, e.g., by directly regulating the activity of enzymes, receptors, ion channels or the like, or by modulating the intracellular concentrations of various signaling molecules. See, e.g., Shigeri et al., Pharmacol. Therapeut., 2001, 91:85+; Curley, et al., Pharmacol. Therapeut., 1999, 82:347+; Curley, et al., Curr. Op. Chem. Bio., 1999, 3:84+; “Caged Compounds” Methods in Enzymology, Marriott, G., Ed, Academic Press, NY, 1998, V. 291; Adams, et al., Annu. Rev. Physiol., 1993, 55:755+; and Bochet, et al., J. Chem. Soc., Perkin 1, 2002, 125+. In various embodiments herein, the compositions and methods comprise photoregulated amino acids. For example, as illustrated in the Example 1 herein, o-nitrobenzyl cysteine (a photocaged amino acid) is the amino acid for some of the O-RS/O-tRNA pairs herein, while Example 2 uses an azobenzyl-Phe which is a photoisomerizable amino acid and will switch isomer form due to light exposure.

“Photoregulated amino acids” are typically, e.g., photosensitive amino acids. Photoregulated amino acids in general are those that are controlled in some fashion by light (e.g., UV, IR, etc.). Thus, for example, if a photoregulated amino acid is comprised within a peptide having biological activity, illumination can alter the amino acid, thereby changing the biological activity of the peptide. Some photoregulated amino acids can comprise “photocaged amino acids,” “photosensitive amino acids,” “photolabile amino acids,” “photoisomerizable,” etc. “Caged species,” such as caged amino acids, or caged peptides, are those trapped inside a larger entity (e.g., molecule) and that are released upon specific illumination. See, e.g., Adams, et al., Annu. Rev. Physiol., 1993, 55:755-784. “Caging” groups of amino acids can inhibit or conceal (e.g., by disrupting bonds which would usually stabilize interactions with target molecules, by changing the hydrophobicity or ionic character of a particular side chain, or by steric hindrance, etc.) biological activity in a molecule, e.g., a peptide comprising such amino acid. “Photoisomerizable” amino acids can switch isomer forms due to light exposure. The different isomers of such amino acids can end up having different interactions with other side chains in a protein upon incorporation. See Example 2. Photoregulated amino acids can thus control the biological activity (either through activation, partial activation, inactivation, partial inactivation, modified activation, etc.) of the peptides in which they are present. See Adams above and other references in this section for further definitions and examples of photoregulated amino acids and molecules.

A number of photoregulated amino acids are known to those in the art and many are available commercially. Methods of attaching and/or associating photoregulating moieties to amino acids are also known. Such photoregulated amino acids in general are amenable to various embodiments herein. It will be appreciated that while a number of possible photoregulating moieties, e.g., photocaging groups and the like, as well as a number of photoregulated amino acids are listed herein, such recitation should not be taken as limiting. Thus, the current invention is also amenable to photoregulating moieties and photoregulated amino acids that are not specifically recited herein.

As stated, a number of methods are optionally applicable to create a photoregulated amino acid. Thus, for example, a photoregulated amino acid, e.g., a photocaged amino acid can be created by protecting its α-amino group with compounds such as BOC (butyloxycarbonyl), and protecting the α-carboxyl group with compounds such as a t-butyl ester. Such protection can be followed by reaction of the amino acid side chain with a photolabile caging group such as 2-nitrobenzyl, in a reactive form such as 2-nitrobenzylchloroformate, α-carboxyl 2-nitrobenzyl bromide methyl ester, or 2-nitrobenzyl diazoethane. After the photolabile cage group is added, the protecting groups can be removed via standard procedures. See, e.g., U.S. Pat. No. 5,998,580.

As another example, lysine residues can be caged using 2-nitrobenzylchloroformate to derivatize the ε-lysine amino group, thus eliminating the positive charge. Alternatively, lysine can be caged by introducing a negative charge into a peptide (which has such lysine) by use of an α-carboxy 2-nitrobenzyloxycarbonyl caging group. Additionally, phosphoserine and phosphothreonine can be caged by treatment of the phosphoamino acid or the phosphopeptide with 1(2-nitrophenyl)diazoethane. See, e.g., Walker et al., Meth Enzymol. 172:288-301, 1989. A number of other amino acids are also easily amenable to standard caging chemistry, for example serine, threonine, histidine, glutamine, asparagine, aspartic acid and glutamic acid. See, e.g., Wilcox et al., J. Org. Chem. 55:1585-1589, 1990). Again, it will be appreciated that recitation of particular photoregulated (amino acids and/or those capable of being converted to photoregulated forms) should not necessarily be taken as limiting.

Amino acid residues can also be made photoregulated (e.g., photosensitive or photolabile) in other fashions. For example, certain amino acid residues can be created wherein irradiation causes cleavage of a peptide backbone that has the particular amino acid residue. For example a photolabile glycine, 2-nitrophenyl glycine, can function in such a manner. See, e.g., Davis, et al., 1973, J. Med. Chem., 16:1043-1045. Irradiation of peptides containing 2-nitrophenylglycine will cleave the peptide backbone between the alpha carbon and the alpha amino group of 2-nitrophenylglycine. Such cleavage strategy is generally applicable to amino acids other than glycine, if the 2-nitrobenzyl group is inserted between the alpha carbon and the alpha amino group.

A large number of photoregulating groups, e.g., caging groups, and a number of reactive compounds used to covalently attach such groups to other molecules such as amino acids, are well known in the art. Examples of photoregulating (e.g., photolabile, caging) groups include, but are not limited to: nitroindolines; N-acyl-7-nitroindolines; phenacyls; hydroxyphenacyl; brominated 7-hydroxycoumarin-4-ylmethyls (e.g., Bhc); benzoin esters; dimethoxybenzoin; meta-phenols; 2-nitrobenzyl; 1-(4,5-dimethoxy-2-nitrophenyl)ethyl (DMNPE); 4,5-dimethoxy-2-nitrobenzyl (DMNB); alpha-carboxy-2-nitrobenzyl (CNB); 1-(2-nitrophenyl)ethyl (NPE); 5-carboxymethoxy-2-nitrobenzyl (CMNB); (5-carboxymethoxy-2-nitrobenzyl)oxy)carbonyl; (4,5-dimethoxy-2-nitrobenzyl)oxy)carbonyl; desoxybenzoinyl; and the like. See, e.g., U.S. Pat. No. 5,635,608 to Haugland and Gee (Jun. 3, 1997) entitled “α-carboxy caged compounds” Neuro 19, 465 (1997); J Physiol 508.3, 801 (1998); Proc Natl Acad Sci USA 1988 September, 85(17):6571-5; J Biol Chem 1997 Feb. 14, 272(7):4172-8; Neuron 20, 619-624, 1998; Nature Genetics, vol. 28:2001:317-325; Nature, vol. 392, 1998:936-941; Pan, P., and Bayley, H. “Caged cysteine and thiophosphoryl peptides” FEBS Letters 405:81-85 (1997); Pettit et al. (1997) “Chemical two-photon uncaging: a novel approach to mapping glutamate receptors” Neuron 19:465-471; Furuta et al. (1999) “Brominated 7-hydroxycoumarin-4-ylmethyls: novel photolabile protecting groups with biologically useful cross-sections for two photon photolysis” Proc. Natl. Acad. Sci. 96(4):1193-1200; Zou et al. “Catalytic subunit of protein kinase A caged at the activating phosphothreonine” J. Amer. Chem. Soc. (2002) 124:8220-8229; Zou et al. “Caged Thiophosphotyrosine Peptides” Angew. Chem. Int. Ed. (2001) 40:3049-3051; Conrad I I et al. “p-Hydroxyphenacyl Phototriggers: The reactive Excited State of Phosphate Photorelease” J. Am. Chem. Soc. (2000) 122:9346-9347; Conrad I I et al. “New Phototriggers 10: Extending the π,π* Absorption to Release Peptides in Biological Media” Org. Lett. (2000) 2:1545-1547; Givens et al. “A New Phototriggers 9: p-Hydroxyphenacyl as a C-Terminus Photoremovable Protecting Group for Oligopeptides” J. Am. Chem. Soc. (2000) 122:2687-2697; Bishop et al. “40-Aminomethyl-2,20-bipyridyl-4-carboxylic Acid (Abc) and Related Derivatives: Novel Bipyridine Amino Acids for the Solid-Phase Incorporation of a Metal Coordination Site Within a Peptide Backbone” Tetrahedron (2000) 56:4629-4638; Ching et al. “Polymers As Surface-Based Tethers with Photolytic triggers Enabling Laser-Induced Release/Desorption of Covalently Bound Molecules” Bioconjugate Chemistry (1996) 7:525-8; BioProbes Handbook, 2002 from Molecular Probes, Inc.; and Handbook of Fluorescent Probes and Research Products, Ninth Edition or Web Edition, from Molecular Probes, Inc, as well as the references herein. Many compounds, kits, etc. for use in caging various molecules are commercially available, e.g., from Molecular Probes, Inc. (www.molecularprobes.com). Additional references are found in, e.g., Merrifield, Science 232:341 (1986) and Corrie, J. E. T. and Trentham, D. R. (1993) In: Biological Applications of Photochemical Switches, ed., Morrison, H., John Wiley and Sons, Inc. New York, pp. 243-305. Examples of suitable photosensitive caging groups include, but are not limited to, 2-nitrobenzyl, benzoin esters, N-acyl-7-nitindolines, meta-phenols, and phenacyls.

In some embodiments, a photoregulating (e.g., caging) group can optionally comprise a first binding moiety, which can bind to a second binding moiety. For example, a commercially available caged phosphoramidite [1-N-(4,4′-Dimethoxytrityl)-5-(6-biotinamidocaproamidomethyl)-1-(2-nitrophenyl)-ethyl]-2-cyanoethyl-(N,N-diisopropyl)-phosphoramidite (PC Biotin Phosphoramadite, from Glen Research Corp., www.glenres.com) comprises a photolabile group and a biotin (the first binding moiety). A second binding moiety, e.g., streptavidin or avidin, can thus be bound to the caging group, increasing its bulkiness and its effectiveness at caging. In certain embodiments, a caged component comprises two or more caging groups each comprising a first binding moiety, and the second binding moiety can bind two or more first binding moieties simultaneously. For example, the caged component can comprise at least two biotinylated caging groups; binding of streptavidin to multiple biotin moieties on multiple caged component molecules links the caged components into a large network. Cleavage of the photolabile group attaching the biotin to the component results in dissociation of the network.

“Traditional” methods of creating caged polypeptides (including e.g. peptide substrates and proteins such as antibodies or transcription factors) include, e.g., by reacting a polypeptide with a caging compound or by incorporating a caged amino acid during synthesis of a polypeptide. See, e.g., U.S. Pat. No. 5,998,580 to Fay et al. (Dec. 7, 1999) entitled “Photosensitive caged macromolecules”; Kossel et al. (2001) PNAS 98:14702-14707; Trends Plant Sci (1999) 4:330-334; PNAS (1998) 95:1568-1573; J. Am. Chem. Soc. (2002) 124:8220-8229; Pharmacology & Therapeutics (2001) 91:85-92; and Angew. Chem. Int. Ed. Engl. (2001) 40:3049-3051. A photolabile polypeptide linker (e.g., for connecting a protein transduction domain and a sensor, or the like) can, for example, comprise a photolabile amino acid such as that described in U.S. Pat. No. 5,998,580.

Irradiation with light can, e.g., release a side chain residue of an amino acid that is important for activity of the peptide comprising such amino acid. Additionally, in some embodiments, uncaged amino acids can cleave the peptide backbone of the peptide comprising the amino acid and can thus, e.g., open a cyclic peptide to a linear peptide with different biological properties, etc.

Activation of a caged peptide can be done through destruction of a photosensitive caging group on a photoregulated amino acid by any standard method known to those skilled in the art. For example, a photosensitive amino acid can be uncaged or activated by exposure to a suitable conventional light source, such as lasers (e.g., emitting in the UV range or infrared range). Those of skill in the art will be aware of and familiar with a number of additional lasers of appropriate wavelengths and energies as well as appropriate application protocols (e.g., exposure duration, etc.) that are applicable to use with photoregulated amino acids such as those utilized herein. Release of photoregulated caged amino acids allows control of the peptides that comprise such amino acids. Such control can be both in terms of location and in terms of time. For example, focused laser exposure can uncage amino acids in one location, while not uncaging amino acids in other locations.

Those skilled in the art will appreciate a variety of assays can be used for evaluating the activity of a photoregulated amino acid, e.g., the assays described in the examples herein. A wide range of, e.g., cellular function, tissue function, etc. can be assayed before and after the introduction of a peptide comprising a photoregulated amino acid into the cell or tissue as well as after the release of the photoregulated molecule.

The compositions and methods herein can be utilized in a number of aspects. For example, photoregulated amino acids (e.g., in peptides) can deliver therapeutic compositions to discrete locations of a body since the release or activation/deactivation/etc. of the photoregulated amino acid can be localized through targeted light exposure, etc. It will also be appreciated that the methods, structures, and compositions of the invention are applicable to incorporation/use of photoregulated natural amino acids (e.g., ones with photoregulating moieties attached/associated with them).

Recently, over thirty unnatural amino acids have been genetically encoded in both prokaryotic and eukaryotic organisms in response to unique triplet and quadruplet codons. See, e.g., Chin, et al., PNAS, 2002, 99:11020-4; Chin, et al., J. Am. Chem. Soc., 2002, 124:9026-7; Alfonta, et al., J. Am. Chem. Soc., 2003, 125:14662-3; Wang, et al., PNAS, 2003, 100:56-61; Zhang, et al., Science, 2004, 303:371-3; Xie, et al., Nature Biotech, 2004, in press; Wang, et al., Angew. Chem., 2004, in press; Chin, et al., Science, 2003, 301:964-7; and Anderson, et al., PNAS, 2004, 101:4566-71. These include glycosylated amino acids, amino acids with keto, azido, alkynyl, and iodo groups, and photoreactive and redox active amino acids. To further increase the structural diversity of this “expanded” genetic code requires additional unique tRNA/aminoacyl tRNA-synthetase pairs, e.g., such as those shown in the Examples below.

Photochromic and photocleavable groups can be used to spatially and temporally control a variety of biological processes, either by directly regulating the activity of enzymes (see, e.g., Westmark, et al., J. Am. Chem. Soc. 1993, 115:3416-19 and Hohsaka, et al., J. Am. Chem. Soc. 1994, 116:413-4), receptors (see, e.g., Bartels, et al., Proc. Natl. Acad. Sci. USA, 1971, 68:1820-3; Lester, et al., Nature 1977, 266:373-4: Cruz, et al., J. Am. Chem. Soc., 2000, 122:8777-8; and, Pollitt, et al., Angew. Chem. Int. Ed. Engl., 1998, 37:2104-7), or ion channels (see, e.g., Lien, et al., J. Am. Chem. Soc. 1996, 118:12222-3; Borisenko, et al., J. Am. Chem. Soc. 2000, 122:6364-70; and, Banghart, et al., Nat. Neurosci. 2004, 7:1381-6.), or by modulating the intracellular concentrations of various signaling molecules (see, e.g., Adams, et al., Annu. Rev. Physiol. 1993, 55:755-84). In general, this requires the chemical modification of either a protein or small molecule with a photoreactive ligand such as azobenzene or a nitrobenzyl group. The ability to genetically incorporate photoresponsive amino acids into proteins at defined sites directly in living organisms would significantly extend the scope of this technique. See, e.g., Wu, et al., J. Am. Chem. Soc. 2004, 126:14306-7.

Orthogonal Components for Incorporating Photoregulated Amino Acids (o-Nitrobenzyl Cysteine and Azobenzyl-Phe), O-Me-L-Tyrosine, and α-Aminocaprylic Acid

The invention provides compositions and methods of producing orthogonal components for incorporating a photoregulated amino acid (e.g., such as o-nitrobenzyl cysteine and azobenzyl-Phe), O-Me-L-tyrosine, or α-aminocaprylic acid into a growing polypeptide chain in response to a selector codon, e.g., amber codon, stop codon, a nonsense codon, a four or more base codon, etc., e.g., in vivo. For example, the invention provides orthogonal-tRNAs (O-tRNAs), orthogonal aminoacyl-tRNA synthetases (O-RSs) and pairs thereof. Nonlimiting examples of such are seen, e.g., in the examples section below. These pairs can be used to incorporate a photoregulated amino acid (e.g., such as o-nitrobenzyl cysteine or azobenzyl-Phe), O-Me-L-tyrosine, and α-aminocaprylic acid into growing polypeptide chains.

A composition of the invention includes an orthogonal aminoacyl-tRNA synthetase (O-RS), where the O-RS preferentially aminoacylates an O-tRNA with a photoregulated amino acid (e.g., such as o-nitrobenzyl cysteine or azobenzyl-Phe), O-Me-L-tyrosine, or α-aminocaprylic acid. In certain embodiments, the O-RS comprises an amino acid sequence comprising those shown/described in the examples section and Sequence Listing herein, or a conservative variation thereof of any such sequences. In certain embodiments of the invention, the O-RS preferentially aminoacylates the O-tRNA with an efficiency of at least 50% of the efficiency of a polypeptide comprising an amino acid sequence of those shown/described in the examples section herein and/or within the sequence listing.

A composition that includes an O-RS can optionally further include an orthogonal tRNA (O-tRNA), where the O-tRNA recognizes a selector codon. Typically, an O-tRNA of the invention includes at least about, e.g., a 45%, a 50%, a 60%, a 75%, a 80%, or a 90% or more suppression efficiency in the presence of a cognate synthetase in response to a selector codon as compared to suppression efficiency of an O-tRNA comprising or encoded by a polynucleotide sequence as set forth in the sequences and examples herein. In one embodiment, the suppression efficiency of the O-RS and the O-tRNA together is, e.g., 5 fold, 10 fold, 15 fold, 20 fold, 25 fold or more greater than the suppression efficiency of the O-tRNA lacking the O-RS. In one aspect, the suppression efficiency of the O-RS and the O-tRNA together is at least 45% of the suppression efficiency of an orthogonal tyrosyl-tRNA synthetase pair derived from Methanococcus jannaschii, while in another aspect it is at least 45% of the suppression efficiency of an orthogonal leucyl-tRNA synthetase pair derived from E. coli.

A composition that includes an O-tRNA can optionally include a cell (e.g., a prokaryotic (non-eukaryotic) cell, such as an E. coli cell and the like, or a eukaryotic cell such as S. cerevisiae), and/or a translation system.

A cell (e.g., a prokaryotic (non-eukaryotic) cell, or a eukaryotic cell) comprising a translation system is also provided by the invention, where the translation system includes an orthogonal-tRNA (O-tRNA); an orthogonal aminoacyl-tRNA synthetase (O-RS); and, an OMe-L-tyrosine, α-aminocaprylic acid, or a photoregulated amino acid such as o-nitrobenzyl cysteine or azobenzyl-Phe. Typically, the O-RS preferentially aminoacylates the O-tRNA with an efficiency of at least 50% of the efficiency of a polypeptide comprising an amino acid sequence of those sequences herein, e.g., in the examples section below. The O-tRNA recognizes the first selector codon, and the O-RS preferentially aminoacylates the O-tRNA with the OMe-L-tyrosine, α-aminocaprylic acid, or photoregulated amino acid such as o-nitrobenzyl cysteine or azobenzyl-Phe. In one embodiment, the O-tRNA comprises or is encoded by a polynucleotide sequence as described by the examples and sequence listing below, or a complementary polynucleotide sequence thereof. In one embodiment, the O-RS comprises an amino acid sequence as described in the examples and sequence listing (e.g., one or more of SEQ ID NO: 5-17) below, or a conservative variation thereof.

A cell of the invention can optionally further comprise an additional different O-tRNA/O-RS pair and a second unnatural amino acid, e.g., where this O-tRNA recognizes a second selector codon and this O-RS preferentially aminoacylates the O-tRNA with the second unnatural amino acid amino acid. Optionally, a cell of the invention includes a nucleic acid that comprises a polynucleotide that encodes a polypeptide of interest, where the polynucleotide comprises a selector codon that is recognized by the O-tRNA.

In certain embodiments, a cell of the invention includes a prokaryotic cell such as E. coli cell or a eukaryotic cell such as S. cerevisiae that includes an orthogonal-tRNA (O-tRNA), an orthogonal aminoacyl-tRNA synthetase (O-RS), an unnatural amino acid such as an OMe-L-tyrosine, α-aminocaprylic acid, or photoregulated amino acid such as o-nitrobenzyl cysteine or azobenzyl-Phe, and a nucleic acid that comprises a polynucleotide that encodes a polypeptide of interest, where the polynucleotide comprises the selector codon that is recognized by the O-tRNA. In certain embodiments of the invention, the O-RS preferentially aminoacylates the O-tRNA with an efficiency of at least 50% of the efficiency of a polypeptide comprising an amino acid sequence of any listed O-RS sequence herein, e.g., as in the examples and sequence listing herein.

In certain embodiments of the invention, an O-tRNA of the invention comprises or is encoded by a polynucleotide sequence as set forth in the sequences and examples herein, or a complementary polynucleotide sequence thereof. In certain embodiments of the invention, an O-RS comprises an amino acid sequence as set forth in the sequences and examples herein, or a conservative variation thereof. In one embodiment, the O-RS or a portion thereof is encoded by a polynucleotide sequence encoding an amino acid as set forth in the sequences or examples herein, or a complementary polynucleotide sequence thereof.

The O-tRNA and/or the O-RS of the invention can be derived from any of a variety of organisms (e.g., eukaryotic and/or prokaryotic (non-eukaryotic) organisms).

Polynucleotides are also a feature of the invention. A polynucleotide of the invention includes an artificial (e.g., man-made, and not naturally occurring) polynucleotide comprising a nucleotide sequence encoding a polypeptide as set forth in the sequences and examples herein, and/or is complementary to or that polynucleotide sequence. A polynucleotide of the invention can also include a nucleic acid that hybridizes to a polynucleotide described above, under highly stringent conditions, over substantially the entire length of the nucleic acid. A polynucleotide of the invention also includes a polynucleotide that is, e.g., at least 75%, at least 80%, at least 90%, at least 95%, at least 98% or more identical to that of a naturally occurring tRNA or corresponding coding nucleic acid (but a polynucleotide of the invention is other than a naturally occurring tRNA or corresponding coding nucleic acid), where the tRNA recognizes a selector codon, e.g., an amber codon. Artificial polynucleotides that are, e.g., at least 80%, at least 90%, at least 95%, at least 98% or more identical to any of the above and/or a polynucleotide comprising a conservative variation of any the above, are also included in polynucleotides of the invention.

Vectors comprising a polynucleotide of the invention are also a feature of the invention. For example, a vector of the invention can include a plasmid, a cosmid, a phage, a virus, an expression vector, and/or the like. A cell comprising a vector of the invention is also a feature of the invention.

Methods of producing components of an O-tRNA/O-RS pair are also features of the invention. Components produced by these methods are also a feature of the invention. For example, methods of producing at least one tRNA that are orthogonal to a cell (O-tRNA) include generating a library of mutant tRNAs; mutating an anticodon loop of each member of the library of mutant tRNAs to allow recognition of a selector codon, thereby providing a library of potential O-tRNAs, and subjecting to negative selection a first population of cells of a first species, where the cells comprise a member of the library of potential O-tRNAs. The negative selection eliminates cells that comprise a member of the library of potential O-tRNAs that is aminoacylated by an aminoacyl-tRNA synthetase (RS) that is endogenous to the cell. This provides a pool of tRNAs that are orthogonal to the cell of the first species, thereby providing at least one O-tRNA. An O-tRNA produced by the methods of the invention is also provided.

In certain embodiments, the methods further comprise subjecting to positive selection a second population of cells of the first species, where the cells comprise a member of the pool of tRNAs that are orthogonal to the cell of the first species, a cognate aminoacyl-tRNA synthetase, and a positive selection marker. Using the positive selection, cells are selected or screened for those cells that comprise a member of the pool of tRNAs that is aminoacylated by the cognate aminoacyl-tRNA synthetase and that shows a desired response in the presence of the positive selection marker, thereby providing an O-tRNA. In certain embodiments, the second population of cells comprise cells that were not eliminated by the negative selection.

Methods for identifying an orthogonal-aminoacyl-tRNA synthetase that charges an OMe-L-tyrosine, α-aminocaprylic acid, or a photoregulated amino acid such as o-nitrobenzyl cysteine or azobenzyl-Phe onto an O-tRNA are also provided. For example, methods include subjecting to selection a population of cells of a first species, where the cells each comprise: 1) a member of a plurality of aminoacyl-tRNA synthetases (RSs), (e.g., the plurality of RSs can include mutant RSs, RSs derived from a species other than a first species or both mutant RSs and RSs derived from a species other than a first species); 2) the orthogonal-tRNA (O-tRNA) (e.g., from one or more species); and 3) a polynucleotide that encodes a positive selection marker and comprises at least one selector codon.

Cells (e.g., a host cell) are selected or screened for those that show an enhancement in suppression efficiency compared to cells lacking or having a reduced amount of the member of the plurality of RSs. These selected/screened cells comprise an active RS that aminoacylates the O-tRNA. An orthogonal aminoacyl-tRNA synthetase identified by the method is also a feature of the invention.

Methods of producing a protein in a cell (e.g., a prokaryotic (non-eukaryotic) cell, such as a prokaryotic E. coli cell or the like, or a eukaryotic cell, such as S. cerevisiae) with a photoregulated amino acid (e.g., such as o-nitrobenzyl cysteine and azobenzyl-Phe), O-Me-L-tyrosine, or α-aminocaprylic acid at a specified position are also a feature of the invention. For example, a method includes growing, in an appropriate medium, a cell, where the cell comprises a nucleic acid that comprises at least one selector codon and encodes a protein, providing the OMe-L-tyrosine, α-aminocaprylic acid, or photoregulated amino acid such as o-nitrobenzyl cysteine or azobenzyl-Phe, and incorporating the photoregulated amino acid (e.g., o-nitrobenzyl cysteine and azobenzyl-Phe), O-Me-L-tyrosine, or α-aminocaprylic acid into the specified position in the protein during translation of the nucleic acid with the at least one selector codon, thereby producing the protein. The cell further comprises: an orthogonal-tRNA (O-tRNA such as leucyl O-tRNA or tyrosyl-O-tRNA) that functions in the cell and recognizes the selector codon; and, an orthogonal aminoacyl-tRNA synthetase (O-RS, e.g., leucyl O-RS or tyrosyl O-RS) that preferentially aminoacylates the O-tRNA with the OMe-L-tyrosine, α-aminocaprylic acid, or photoregulated amino acid such as o-nitrobenzyl cysteine or azobenzyl-Phe. A protein produced by this method is also a feature of the invention.

The invention also provides compositions that include proteins, where the proteins comprise, e.g., a photoregulated amino acid (e.g., such as o-nitrobenzyl cysteine and azobenzyl-Phe), O-Me-L-tyrosine, or α-aminocaprylic acid. In certain embodiments, the protein comprises an amino acid sequence that is at least 75% identical to that of a known protein, e.g., a therapeutic protein, a diagnostic protein, an industrial enzyme, or portion thereof. Optionally, the composition comprises a pharmaceutically acceptable carrier.

### Novel Synthetase Libraries and Library Screening Methods

The invention provides novel polynucleotide libraries and novel library screening methods that are used in the identification of novel aminoacyl-tRNA synthetase variants that are orthogonal aminoacyl-tRNA synthetases (O-RS) that act in concert with a corresponding orthogonal tRNA (O-tRNA) in a particular host cell. These reagents and methods can be used to identify a desired O-RS species that has the ability to charge its partner O-tRNA with any desired unnatural amino acid.

The synthetase libraries of the invention comprise polynucleotides encoding aminoacyl-tRNA synthetase variants. In certain embodiments, these synthetase variants are derived from Archaea aminoacyl-tRNA synthetase genes. The source of the Archaea aminoacyl-tRNA synthetase sequence is not particularly limited. For example, the source material can be from a polynucleotide encoding a wild-type Methanococcus jannaschii aminoacyl-tRNA synthetase, or from any other Archaea species, for example, Methanobacterium thermoautotrophicum (Mt), or Pyrococcus horikoshii (Ph), or any other Archaea species. In yet other embodiments, the synthetase variants are derived from eubacterial aminoacyl-tRNA synthetase genes. The source of the eubacterial aminoacyl-tRNA synthetase sequence is not particularly limited. For example the source material can be from a polynucleotide encoding a wild-type E. coli aminoacyl-tRNA synthetase, or from any other eubacterial species, for example, Thermus thermophilus.

As described in the Examples herein, a polynucleotide encoding a wild-type Methanococcus jannaschii aminoacyl-tRNA synthetase that charges a cognate tRNA with tyrosine (MjTyr-RS) can be used as starting material to generate the variant synthetase library. Also described within the Examples is a polynucleotide encoding a wild-type E. coli aminoacyl tRNA synthetase that charges a cognate tRNA with leucine (EcLeu-RS) used as a starting material to generate a variant synthetase library. However, it is not intended that the invention be limited to a tyrosyl-specific aminoacyl-tRNA synthetase as starting material. Any aminoacyl-tRNA synthetase can be used. In some embodiments, the particular aminoacyl-tRNA synthetase chosen as starting material for the library construction can be influenced by the particular unnatural amino acid of interest. For example, if the unnatural amino acid of interest has an aromatic R-group, it is advantageous to construct the variant synthetase library using a starting material polynucleotide encoding an RS specific for tyrosine or phenylalanine.

The variant RS library is generated by randomizing the codons at the amino acid positions that form the amino acid binding pocket in the RS. This information is typically obtained by analysis of the crystal structure of the RS. For example, based on the crystal structure of the Methanococcus jannaschii TyrRS-tRNA(Tyr)-L-tyrosine complex (see, e.g., Kobayashi et al., Nat. Struct. Biol., 10:425-432 (2003)), specific positions within the amino acid binding pocket of the MjTyr-RS synthetase were randomized. These residues were Tyr-32, Leu-65, Phe-108, Gln-109, Asp-158 and Leu-162 in the tyrosine binding site. This selectively randomized library was hoped to be advantageous for the selection of synthetase variants that charge a tRNA with an unnatural amino acid having an aromatic side chain (for example but not limited to azobenzyl-phenylalanine (also termed “azobenzyl-Phe” elsewhere herein), and where the unnatural amino acid is excluded. Randomization of the targeted codons can be accomplished by randomizing one, two or all three nucleotide positions in the codon.

The amino acid positions in an aminoacyl-tRNA synthetase selected for randomization are not strictly limited to these six amino acid positions in MjTyr-RS. For example, in the case where a synthetase from a species other than Methanococcus jannaschii is used in the mutagenesis (e.g., an MjTyr-RS ortholog), the amino acid sequence of the synthetase ortholog may not be 100% identical with the MjTyr-RS. However, the Tyr-RS structure is conserved and can be predicted for the MjTyr-RS ortholog. The amino acid positions in the MjTyr-RS ortholog that spatially correspond to the amino acids that lie within the amino acid binding pocket in the M. jannaschii Tyr-RS (e.g., positions Tyr-32, Leu-65, Phe-108, Gln-109, Asp-158 and Leu-162) can be determined based on the known crystal structure of the M. jannaschii Tyr-RS. For example, the leucine that resides at position 65 in MjTyr-RS (see SEQ ID NO: 4) may spatially correspond to a different leucine position in a orthologous Tyr-RS, for example, a Tyr-RS derived from Methanobacterium thermoautotrophicum or a Tyr-RS derived from Pyrococcus horikoshii. As will be appreciated a corresponding logic is present in the construction of the libraries of Example 1 comprising an E. coli RS.

It is preferable to maximize diversity in the variant synthetase library. For example, in some aspects, the variant RS library can have 10⁹ or more unique polynucleotide members that encode synthetase variants.

As recognized in the art, libraries of polynucleotide sequences are frequently manipulated (e.g., propagated, expanded, cultured or plated) following their transformation into a suitable host. In some aspects, such as those described in the Examples herein, a suitable host cell can be a eubacterial cell such as E. coli. However, suitable eubacterial hosts are not limited to E. coli, as other eubacterial hosts can also find use with the invention. Additionally, for various other embodiments, a eukaryote such as S. cerevisiae can comprise a suitable host cell.

The invention also provides methods for screening libraries such as the libraries described above for the purpose of identifying a desired orthogonal aminoacyl-tRNA synthetase (O-RS) that incorporates an unnatural amino acid of interest. These methods comprise detecting those variant synthetases in the library that have the ability to charge a cognate O-tRNA with the unnatural amino acid of interest to the exclusion of other natural amino acids. The selection of the preferred variants typically uses a combination of both positive selection steps and negative selection steps, although in some embodiments only a positive selection scheme can be employed.

In one type of positive selection scheme, the variant synthetase library is plated as transformed host cells that also harbor a cognate tRNA and a co-transformed chloramphenicol acetyltransferase gene having a selector codon (e.g., an internal amber selector codon). Other embodiments can comprise host cells with screening/selection as shown in Example 1 (e.g., using growth in a uracil free media or in a histidine free media supplemented with aminotriazole). In this positive selection scheme, cell survival is dependent on the suppression of the amber codon when the cells are grown in the presence of the unnatural amino acid (e.g., azobenzyl-phenylalanine) and chloramphenicol.

The positive selection scheme can be optionally combined with a negative selection scheme to eliminate those positively selected synthetase clones that permit charging of the tRNA with a natural amino acid. For example, clonal variant synthetase candidates identified following the positive selection are transformed into cells containing the orthogonal tRNA and a gene encoding the toxic barnase protein with one or more selector codons or by expression of ura3 gene product in the presence of fluorootic acid. These cells are grown in the absence of unnatural amino acid (e.g., azobenzyl-phenylalanine). Any synthetase variant clones that fail to grow under these conditions in the absence of unnatural amino acid are removed from further analysis, as these synthetase clones presumably permit charging of the O-tRNA with natural amino acid, or somehow circumvent the selector codon to permit expression of the toxic reporter in the absence of the unnatural amino acid. In some embodiments of this strategy, multiple rounds of both positive and negative selection are conducted on the variant synthetase candidates.

The invention is not limited to the use of the chloramphenicol acetyltransferase gene as a positive selectable marker, nor is the invention limited to the use of barnase as a negative selection marker. One of skill in the art will recognize alternative selection strategies that can readily be applied to monitor expression or lack of expression of any given gene product. These alternative selection reagents and methods fall within the scope of the present invention.

### Nucleic Acid and Polypeptide Sequence and Variants

As described above and below, the invention provides for nucleic acid polynucleotide sequences, e.g., O-tRNAs and O-RSs, and polypeptide amino acid sequences, e.g., O-RSs, and, e.g., compositions, systems and methods comprising said sequences. Examples of said sequences, e.g., O-tRNAs and O-RSs are disclosed herein (see the sequences and examples herein). However, one of skill in the art will appreciate that the invention is not limited to those exact sequences, e.g., as in the Examples and sequence listing. One of skill will appreciate that the invention also provides, e.g., many related and unrelated sequences with the functions described herein, e.g., encoding an appropriate O-tRNA or an O-RS.

The invention provides polypeptides (O-RSs) and polynucleotides, e.g., O-tRNA, polynucleotides that encode O-RSs or portions thereof, oligonucleotides used to isolate aminoacyl-tRNA synthetase clones, etc. Polynucleotides of the invention include those that encode proteins or polypeptides of interest of the invention with one or more selector codon. In addition, polynucleotides of the invention include, e.g., a polynucleotide comprising a nucleotide sequence as set forth in the sequences (e.g., SEQ ID NO: 20-32) and examples herein; a polynucleotide that is complementary to or that encodes a polynucleotide sequence thereof. A polynucleotide of the invention also includes a polynucleotide that encodes an amino acid sequence comprising any of those in the sequences (e.g., SEQ ID NO: 5-17) or examples herein. A polynucleotide of the invention also includes a polynucleotide that encodes a polypeptide of the invention. Similarly, an artificial nucleic acid that hybridizes to a polynucleotide indicated above under highly stringent conditions over substantially the entire length of the nucleic acid (and is other than a naturally polynucleotide) is a polynucleotide of the invention. In one embodiment, a composition includes a polypeptide of the invention and an excipient (e.g., buffer, water, pharmaceutically acceptable excipient, etc.). The invention also provides an antibody or antisera specifically immunoreactive with a polypeptide of the invention. An artificial polynucleotide is a polynucleotide that is man made and is not naturally occurring.

A polynucleotide of the invention also includes an artificial polynucleotide that is, e.g., at least 75%, at least 80%, at least 90%, at least 95%, at least 98% or more identical to that of a naturally occurring tRNA, (but is other than a naturally occurring tRNA) or any tRNA or coding nucleic acid thereof in a listing or example herein. A polynucleotide also includes an artificial polynucleotide that is, e.g., at least 75%, at least 80%, at least 90%, at least 95%, at least 98% or more identical to that of a naturally occurring tRNA.

In certain embodiments, a vector (e.g., a plasmid, a cosmid, a phage, a virus, etc.) comprises a polynucleotide of the invention. In one embodiment, the vector is an expression vector. In another embodiment, the expression vector includes a promoter operably linked to one or more of the polynucleotides of the invention. In another embodiment, a cell comprises a vector that includes a polynucleotide of the invention.

One of skill will also appreciate that many variants of the disclosed sequences are included in the invention. For example, conservative variations of the disclosed sequences that yield a functionally similar sequence are included in the invention. Variants of the nucleic acid polynucleotide sequences, wherein the variants hybridize to at least one disclosed sequence and recognize a selector codon, are considered to be included in the invention. Unique subsequences of the sequences disclosed herein, as determined by, e.g., standard sequence comparison techniques, are also included in the invention.

Conservative Variations

Owing to the degeneracy of the genetic code, “silent substitutions” (i.e., substitutions in a nucleic acid sequence which do not result in an alteration in an encoded polypeptide) are an implied feature of every nucleic acid sequence which encodes an amino acid. Similarly, “conservative amino acid substitutions,” in one or a few amino acids in an amino acid sequence are substituted with different amino acids with highly similar properties, are also readily identified as being highly similar to a disclosed construct. Such conservative variations of each disclosed sequence are a feature of the present invention.

“Conservative variations” of a particular nucleic acid sequence refers to those nucleic acids which encode identical or essentially identical amino acid sequences, or, where the nucleic acid does not encode an amino acid sequence, to essentially identical sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid. Thus, “conservative variations” of a listed polypeptide sequence of the present invention include substitutions of a small percentage, typically less than 5%, more typically less than 2% or 1%, of the amino acids of the polypeptide sequence, with an amino acid of the same conservative substitution group. Finally, the addition of sequences which do not alter the encoded activity of a nucleic acid molecule, such as the addition of a non-functional sequence, is a conservative variation of the basic nucleic acid.

Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the polypeptide molecule. The following sets forth example groups that contain natural amino acids of like chemical properties, where substitutions within a group is a “conservative substitution.”

TABLE 1

| Nonpolar | | | | |
|---|---|---|---|---|
| and/or | Polar, | | Positively | Negatively |
| Aliphatic | Uncharged | Aromatic | Charged | Charged |
| Side Chains | Side Chains | Side Chains | Side Chains | Side Chains |
| Glycine | Serine | Phenylalanine | Lysine | Aspartate |
| Alanine | Threonine | Tyrosine | Arginine | Glutamate |
| Valine | Cysteine | Tryptophan | Histidine | |
| Leucine | Methionine | | | |
| Isoleucine | Asparagine | | | |
| Proline | Glutamine | | | |

Nucleic Acid Hybridization

Comparative hybridization can be used to identify nucleic acids of the invention, such as those in the sequences and examples herein, including conservative variations of nucleic acids of the invention, and this comparative hybridization method is one method of distinguishing nucleic acids of the invention from unrelated nucleic acids. In addition, target nucleic acids which hybridize to a nucleic acid represented by those of the sequence listing (e.g., SEQ ID NO: 20-32) and examples herein under high, ultra-high and ultra-ultra high stringency conditions are a feature of the invention. Examples of such nucleic acids include those with one or a few silent or conservative nucleic acid substitutions as compared to a given nucleic acid sequence.

A test nucleic acid is said to specifically hybridize to a probe nucleic acid when it hybridizes at least one half as well to the probe as to the perfectly matched complementary target, i.e., with a signal to noise ratio at least one half as high as hybridization of the probe to the target under conditions in which the perfectly matched probe binds to the perfectly matched complementary target with a signal to noise ratio that is at least about 5×−10× as high as that observed for hybridization to any of the unmatched target nucleic acids.

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, N.Y.), as well as in Ausubel, infra. Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) provide details on the synthesis, labeling, detection and quantification of DNA and RNA, including oligonucleotides.

An example of stringent hybridization conditions for hybridization of complementary nucleic acids which have more than 100 complementary residues on a filter in a Southern or northern blot is 50% formalin with 1 mg of heparin at 42° C., with the hybridization being carried out overnight. An example of stringent wash conditions is a 0.2×SSC wash at 65° C. for 15 minutes (see, Sambrook, infra for a description of SSC buffer). Often the high stringency wash is preceded by a low stringency wash to remove background probe signal. An example low stringency wash is 2×SSC at 40° C. for 15 minutes. In general, a signal to noise ratio of 5× (or higher) than that observed for an unrelated probe in the particular hybridization assay indicates detection of a specific hybridization.

“Stringent hybridization wash conditions” in the context of nucleic acid hybridization experiments such as Southern and northern hybridizations are sequence dependent, and are different under different environmental parameters. An extensive guide to the hybridization of nucleic acids is found in Tijssen (1993), supra, and in Hames and Higgins, 1 and 2. Stringent hybridization and wash conditions can easily be determined empirically for any test nucleic acid. For example, in determining stringent hybridization and wash conditions, the hybridization and wash conditions are gradually increased (e.g., by increasing temperature, decreasing salt concentration, increasing detergent concentration and/or increasing the concentration of organic solvents such as formalin in the hybridization or wash), until a selected set of criteria are met. For example, in highly stringent hybridization and wash conditions, the hybridization and wash conditions are gradually increased until a probe binds to a perfectly matched complementary target with a signal to noise ratio that is at least 5× as high as that observed for hybridization of the probe to an unmatched target.

“Very stringent” conditions are selected to be equal to the thermal melting point (Tₘ) for a particular probe. The Tₘ is the temperature (under defined ionic strength and pH) at which 50% of the test sequence hybridizes to a perfectly matched probe. For the purposes of the present invention, generally, “highly stringent” hybridization and wash conditions are selected to be about 5° C. lower than the Tₘ for the specific sequence at a defined ionic strength and pH.

“Ultra high-stringency” hybridization and wash conditions are those in which the stringency of hybridization and wash conditions are increased until the signal to noise ratio for binding of the probe to the perfectly matched complementary target nucleic acid is at least 10× as high as that observed for hybridization to any of the unmatched target nucleic acids. A target nucleic acid which hybridizes to a probe under such conditions, with a signal to noise ratio of at least one half that of the perfectly matched complementary target nucleic acid is said to bind to the probe under ultra-high stringency conditions.

Similarly, even higher levels of stringency can be determined by gradually increasing the hybridization and/or wash conditions of the relevant hybridization assay. For example, those in which the stringency of hybridization and wash conditions are increased until the signal to noise ratio for binding of the probe to the perfectly matched complementary target nucleic acid is at least 10×, 20×, 50×, 100×, or 500× or more as high as that observed for hybridization to any of the unmatched target nucleic acids. A target nucleic acid which hybridizes to a probe under such conditions, with a signal to noise ratio of at least one half that of the perfectly matched complementary target nucleic acid is said to bind to the probe under ultra-ultra-high stringency conditions.

Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

Unique Subsequences

In one aspect, the invention provides a nucleic acid that comprises a unique subsequence in a nucleic acid selected from the sequences of O-tRNAs and O-RSs disclosed herein (see, e.g., examples and sequence listing herein). The unique subsequence is unique as compared to a nucleic acid corresponding to any previously known O-tRNA or O-RS nucleic acid sequence. Alignment can be performed using, e.g., BLAST set to default parameters. Any unique subsequence is useful, e.g., as a probe to identify the nucleic acids of the invention.

Similarly, the invention includes a polypeptide which comprises a unique subsequence in a polypeptide selected from the sequences of O-RSs disclosed herein (see, e.g., examples and sequence listing herein). Here, the unique subsequence is unique as compared to a polypeptide corresponding to any previously known RS sequence.

The invention also provides target nucleic acids which hybridize under stringent conditions to a unique coding oligonucleotide which encodes a unique subsequence in a polypeptide selected from the sequences of O-RSs wherein the unique subsequence is unique as compared to a polypeptide corresponding to any of the control polypeptides (e.g., parental sequences from which synthetases of the invention were derived, e.g., by mutation). Unique sequences are determined as noted above.

Sequence Comparison Identity, and Homology

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of skill) or by visual inspection.

The phrase “substantially identical,” in the context of two nucleic acids or polypeptides (e.g., DNAs encoding an O-tRNA or O-RS, or the amino acid sequence of an O-RS) refers to two or more sequences or subsequences that have at least about 60%, about 80%, about 90-95%, about 98%, about 99% or more nucleotide or amino acid residue identity, when compared and aligned for maximum correspondence, as measured using a sequence comparison algorithm or by visual inspection. Such “substantially identical” sequences are typically considered to be “homologous,” without reference to actual ancestry. Preferably, the “substantial identity” exists over a region of the sequences that is at least about 50 residues in length, more preferably over a region of at least about 100 residues, and most preferably, the sequences are substantially identical over at least about 150 residues, or over the full length of the two sequences to be compared.

Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. For example, any naturally occurring nucleic acid can be modified by any available mutagenesis method to include one or more selector codon. When expressed, this mutagenized nucleic acid can encode a polypeptide comprising one or more a photoregulated amino acid (e.g., such as o-nitrobenzyl cysteine and azobenzyl-Phe), O-Me-L-tyrosine, or α-aminocaprylic acid, e.g. unnatural amino acid. The mutation process can, of course, additionally alter one or more standard codon, thereby changing one or more standard amino acid in the resulting mutant protein as well. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95%, or 99% or more, can also be used to establish homology. Methods for determining sequence similarity percentages (e.g., BLASTP and BLASTN using default parameters) are described herein and are generally available.

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection (see generally Current Protocols in Molecular Biology, Ausubel et al., eds., Current Protocols, a joint venture between Greene Publishing Associates, Inc. and John Wiley & Sons, Inc., supplemented through 2004).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Mutagenesis and Other Molecular Biology Techniques

Polynucleotide and polypeptides of the invention and used in the invention can be manipulated using molecular biological techniques. General texts which describe molecular biological techniques include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2001 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2003) (“Ausubel”)). These texts describe mutagenesis, the use of vectors, promoters and many other relevant topics related to, e.g., the generation of genes that include selector codons for production of proteins that include an OMe-L-tyrosine, or an α-aminocaprylic acid, or a photoregulated amino acid such as o-nitrobenzyl cysteine or azobenzyl-Phe, orthogonal tRNAs, orthogonal synthetases, and pairs thereof.

Various types of mutagenesis are used in the invention, e.g., to mutate tRNA molecules, to produce libraries of tRNAs, to produce libraries of synthetases, to insert selector codons that encode a photoregulated amino acid (e.g., such as o-nitrobenzyl cysteine and azobenzyl-Phe), or O-Me-L-tyrosine, or α-aminocaprylic acid in a protein or polypeptide of interest. They include but are not limited to site-directed, random point mutagenesis, homologous recombination, DNA shuffling or other recursive mutagenesis methods, chimeric construction, mutagenesis using uracil containing templates, oligonucleotide-directed mutagenesis, phosphorothioate-modified DNA mutagenesis, mutagenesis using gapped duplex DNA or the like, or any combination thereof. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, is also included in the present invention. In one embodiment, mutagenesis can be guided by known information of the naturally occurring molecule or altered or mutated naturally occurring molecule, e.g., sequence, sequence comparisons, physical properties, crystal structure or the like.

Host cells are genetically engineered (e.g., transformed, transduced or transfected) with the polynucleotides of the invention or constructs which include a polynucleotide of the invention, e.g., a vector of the invention, which can be, for example, a cloning vector or an expression vector. For example, the coding regions for the orthogonal tRNA, the orthogonal tRNA synthetase, and the protein to be derivatized are operably linked to gene expression control elements that are functional in the desired host cell. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both (e.g., shuttle vectors) and selection markers for both prokaryotic and eukaryotic systems. Vectors are suitable for replication and/or integration in prokaryotes, eukaryotes, or preferably both. See Giliman & Smith, Gene 8:81 (1979); Roberts, et al., Nature, 328:731 (1987); Schneider, B., et al., Protein Expr. Purif 6435:10 (1995); Ausubel, Sambrook, Berger (all supra). The vector can be, for example, in the form of a plasmid, a bacterium, a virus, a naked polynucleotide, or a conjugated polynucleotide. The vectors are introduced into cells and/or microorganisms by standard methods including electroporation (From et al., Proc. Natl. Acad. Sci. USA 82, 5824 (1985), infection by viral vectors, high velocity ballistic penetration by small particles with the nucleic acid either within the matrix of small beads or particles, or on the surface (Klein et al., Nature 327, 70-73 (1987)), and/or the like.

A catalogue of bacteria and bacteriophages useful for cloning is provided, e.g., by the ATCC, e.g., The ATCC Catalogue of Bacteria and Bacteriophage (1996) Ghema et al. (eds) published by the ATCC. Additional basic procedures for sequencing, cloning and other aspects of molecular biology and underlying theoretical considerations are also found in Sambrook (supra), Ausubel (supra), and in Watson et al. (1992) Recombinant DNA Second Edition Scientific American Books, NY. In addition, essentially any nucleic acid (and virtually any labeled nucleic acid, whether standard or non-standard) can be custom or standard ordered from any of a variety of commercial sources, such as the Midland Certified Reagent Company (Midland, Tex. at mcrc.com), The Great American Gene Company (Ramona, Calif. available on the World Wide Web at genco.com), ExpressGen Inc. (Chicago, Ill. available on the World Wide Web at expressgen.com), Operon Technologies, Inc. (Alameda, Calif.) and many others.

The engineered host cells can be cultured in conventional nutrient media modified as appropriate for such activities as, for example, screening steps, activating promoters or selecting transformants. These cells can optionally be cultured into transgenic organisms. Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla.

### Proteins and Polypeptides of Interest

Proteins or polypeptides of interest, e.g., having at least one photoregulated amino acid (e.g., such as o-nitrobenzyl cysteine and azobenzyl-Phe), or O-Me-L-tyrosine, or α-aminocaprylic acid, are a feature of the invention, as are polypeptides comprising two or more different unnatural amino acids. An excipient (e.g., a pharmaceutically acceptable excipient) can also be present with the protein. Optionally, a protein of the invention will include a post-translational modification.

Methods of producing a protein in a cell with an OMe-L-tyrosine, α-aminocaprylic acid, or photoregulated amino acid such as o-nitrobenzyl cysteine or azobenzyl-Phe or other unnatural amino acid at a specified position are also a feature of the invention. For example, a method includes growing, in an appropriate medium, the cell, where the cell comprises a nucleic acid that comprises at least one selector codon and encodes a protein; and, providing the photoregulated amino acid (e.g., such as o-nitrobenzyl cysteine or azobenzyl-Phe), O-Me-L-tyrosine, or α-aminocaprylic acid or other unnatural amino acid; where the cell further comprises: an orthogonal-tRNA (O-tRNA) that functions in the cell and recognizes the selector codon; and, an orthogonal aminoacyl-tRNA synthetase (O-RS) that preferentially aminoacylates the O-tRNA with the photoregulated amino acid (e.g., such as o-nitrobenzyl cysteine and azobenzyl-Phe), O-Me-L-tyrosine, α-aminocaprylic acid or other unnatural amino acid. In certain embodiments, the O-tRNA comprises at least about, e.g., a 45%, a 50%, a 60%, a 75%, a 80%, or a 90% or more suppression efficiency in the presence of a cognate synthetase in response to the selector codon as compared to the O-tRNA comprising or encoded by a polynucleotide sequence as set forth in the sequences and examples herein. A protein produced by this method is also a feature of the invention.

The invention also provides compositions that include proteins, where the proteins comprise an OMe-L-tyrosine, α-aminocaprylic acid, or photoregulated amino acid such as o-nitrobenzyl cysteine or azobenzyl-Phe. In certain embodiments, the protein comprises an amino acid sequence that is at least 75% identical to that of a target protein such as a therapeutic protein, a diagnostic protein, an industrial enzyme, or portion thereof, e.g., differing from the target protein by introduction of one or more unnatural amino acid such as a photoregulated amino acid (e.g., such as o-nitrobenzyl cysteine and azobenzyl-Phe), O-Me-L-tyrosine, or α-aminocaprylic acid.

The compositions of the invention and compositions made by the methods of the invention optionally are present in a cell. The O-tRNA/O-RS pairs or individual components of the invention can then be used in a host system's translation machinery, which results in a photoregulated amino acid (e.g., such as o-nitrobenzyl cysteine or azobenzyl-Phe), O-Me-L-tyrosine, or α-aminocaprylic acid being incorporated into a protein. International Application Number PCT/US2004/011786, filed Apr. 16, 2004, entitled “Expanding the Eukaryotic Genetic Code;” and, WO 2002/085923, entitled “IN VIVO INCORPORATION OF UNNATURAL AMINO ACIDS” describe processes amenable to the current invention, and are incorporated herein by reference. For example, when an O-tRNA/O-RS pair is introduced into a host, e.g., Escherichia coli or S. cerevisiae, the pair leads to the in vivo incorporation of a synthetic amino acid, such as an OMe-L-tyrosine, α-aminocaprylic acid, or photoregulated amino acid such as o-nitrobenzyl cysteine or azobenzyl-Phe, which can be exogenously added to the growth medium, into a protein, in response to a selector codon. Optionally, the compositions of the present invention can be in an in vitro translation system, or in an in vivo system(s).

A cell of the invention provides the ability to synthesize proteins that comprise unnatural amino acids in large useful quantities. In one aspect, the composition optionally includes, e.g., at least 10 micrograms, at least 50 micrograms, at least 75 micrograms, at least 100 micrograms, at least 200 micrograms, at least 250 micrograms, at least 500 micrograms, at least 1 milligram, at least 10 milligrams or more of the protein that comprises a photoregulated amino acid (e.g., such as o-nitrobenzyl cysteine or azobenzyl-Phe), O-Me-L-tyrosine, or α-aminocaprylic acid or multiple unnatural amino acids, or an amount that can be achieved with in vivo protein production methods (details on recombinant protein production and purification are, e.g., provided herein). In another aspect, the protein is optionally present in the composition at a concentration of, e.g., at least 10 micrograms of protein per liter, at least 50 micrograms of protein per liter, at least 75 micrograms of protein per liter, at least 100 micrograms of protein per liter, at least 200 micrograms of protein per liter, at least 250 micrograms of protein per liter, at least 500 micrograms of protein per liter, at least 1 milligram of protein per liter, or at least 10 milligrams of protein per liter or more, in, e.g., a cell lysate, a buffer, a pharmaceutical buffer, or other liquid suspension (e.g., in a volume of, e.g., anywhere from about 1 nL to about 100 L). The production of large quantities (e.g., greater that that typically possible with other methods, e.g., in vitro translation) of a protein in a cell including at least one photoregulated amino acid (e.g., o-nitrobenzyl cysteine or azobenzyl-Phe), O-Me-L-tyrosine, or α-aminocaprylic acid is a feature of the invention.

The incorporation of an OMe-L-tyrosine, α-aminocaprylic acid, or photoregulated amino acid such as o-nitrobenzyl cysteine or azobenzyl-Phe or other unnatural amino acids can be done to, e.g., tailor changes in protein structure and/or function, e.g., to change size, acidity, nucleophilicity, hydrogen bonding, hydrophobicity, accessibility of protease target sites, target to a moiety (e.g., for a protein array), etc. Proteins that include a photoregulated amino acid (e.g., such as o-nitrobenzyl cysteine or azobenzyl-Phe), O-Me-L-tyrosine, or α-aminocaprylic acid can have enhanced or even entirely new catalytic or physical properties. For example, the following properties are optionally modified by inclusion of an OMe-L-tyrosine, α-aminocaprylic acid, or photoregulated amino acid such as o-nitrobenzyl cysteine or azobenzyl-Phe or other unnatural amino acid into a protein: toxicity, biodistribution, structural properties, spectroscopic properties, chemical and/or photochemical properties, catalytic ability, half-life (e.g., serum half-life), ability to react with other molecules, e.g., covalently or noncovalently, and the like. The compositions including proteins that include at least one photoregulated amino acid (e.g., such as o-nitrobenzyl cysteine or azobenzyl-Phe), O-Me-L-tyrosine, or α-aminocaprylic acid are useful for, e.g., novel therapeutics, diagnostics, catalytic enzymes, industrial enzymes, binding proteins (e.g., antibodies), and e.g., the study of protein structure and function. See, e.g., Dougherty, (2000) Unnatural Amino Acids as Probes of Protein Structure and Function, Current Opinion in Chemical Biology, 4:645-652. In addition, one or more unnatural amino acids can be incorporated into a polypeptide to provide a molecular tag, e.g., to fix the polypeptide to a solid support. See e.g., “PROTEIN ARRAYS” by Wang and Schultz, filed Dec. 22, 2003, Attorney Docket Number 54-000810PC for an extended discussion of methods of making arrays using polypeptides that comprise unnatural amino acids.

In one aspect of the invention, a composition includes at least one protein with at least one, e.g., at least two, at least three, at least four, at least five, at least six, at least seven, at least eight, at least nine, or at least ten or more unnatural amino acids, e.g., a photoregulated amino acid (e.g., such as o-nitrobenzyl cysteine or azobenzyl-Phe), O-Me-L-tyrosine, or α-aminocaprylic acid and/or other unnatural amino acids. The unnatural amino acids can be the same or different, e.g., there can be 1, 2, 3, 4, 5, 6, 7, 8, 9, or 10 or more different sites in the protein that comprise 1, 2, 3, 4, 5, 6, 7, 8, 9, or 10 or more different unnatural amino acids. In another aspect, a composition includes a protein with at least one, but fewer than all, of a particular amino acid present in the protein is substituted with the OMe-L-tyrosine, α-aminocaprylic acid, or photoregulated amino acid such as o-nitrobenzyl cysteine or azobenzyl-Phe. For a given protein with more than one unnatural amino acids, the unnatural amino acids can be identical or different (e.g., the protein can include two or more different types of unnatural amino acids, or can include two of the same unnatural amino acid). For a given protein with more than two unnatural amino acids, the unnatural amino acids can be the same, different or a combination of a multiple unnatural amino acid of the same kind with at least one different unnatural amino acid.

Essentially any protein (or portion thereof) that includes an unnatural amino acid such as a photoregulated amino acid (e.g., such as o-nitrobenzyl cysteine or azobenzyl-Phe), O-Me-L-tyrosine, or α-aminocaprylic acid, or that encodes multiple different unnatural amino acids (and any corresponding coding nucleic acid, e.g., which includes one or more selector codons) can be produced using the compositions and methods herein. No attempt is made to identify the hundreds of thousands of known proteins, any of which can be modified to include one or more unnatural amino acid, e.g., by tailoring any available mutation methods to include one or more appropriate selector codon in a relevant translation system. Common sequence repositories for known proteins include GenBank EMBL, DDBJ and the NCBI. Other repositories can easily be identified by searching the internet.

Typically, the proteins are, e.g., at least 60%, at least 70%, at least 75%, at least 80%, at least 90%, at least 95%, or at least 99% or more identical to any available protein (e.g., a therapeutic protein, a diagnostic protein, an industrial enzyme, or portion thereof, and the like), and they comprise one or more unnatural amino acid. Examples of therapeutic, diagnostic, and other proteins that can be modified to comprise one or more photoregulated amino acid (e.g., such as o-nitrobenzyl cysteine and azobenzyl-Phe), O-Me-L-tyrosine, or α-aminocaprylic acid can be found, but not limited to, those in International Application Number PCT/US2004/011786, filed Apr. 16, 2004, entitled “Expanding the Eukaryotic Genetic Code;” and, WO 2002/085923, entitled “IN VIVO INCORPORATION OF UNNATURAL AMINO ACIDS.” Examples of therapeutic, diagnostic, and other proteins that can be modified to comprise one or more homoglutamines include, but are not limited to, e.g., Alpha-1 antitrypsin, Angiostatin, Antihemolytic factor, antibodies, Apolipoprotein, Apoprotein, Atrial natriuretic factor, Atrial natriuretic polypeptide, Atrial peptides, C—X—C chemokines (e.g., T39765, NAP-2, ENA-78, Gro-a, Gro-b, Gro-c, IP-10, GCP-2, NAP-4, SDF-1, PF4, MIG), Calcitonin, CC chemokines (e.g., Monocyte chemoattractant protein-1, Monocyte chemoattractant protein-2, Monocyte chemoattractant protein-3, Monocyte inflammatory protein-1 alpha, Monocyte inflammatory protein-1 beta, RANTES, 1309, R83915, R91733, HCC1, T58847, D31065, T64262), CD40 ligand, C-kit Ligand, Collagen, Colony stimulating factor (CSF), Complement factor 5a, Complement inhibitor, Complement receptor 1, cytokines, (e.g., epithelial Neutrophil Activating Peptide-78, GROα/MGSA, GROβ, GROγ, MIP-1α, MIP-1δ, MCP-1), Epidermal Growth Factor (EGF), Erythropoietin (“EPO”), Exfoliating toxins A and B, Factor IX, Factor VII, Factor VIII, Factor X, Fibroblast Growth Factor (FGF), Fibrinogen, Fibronectin, G-CSF, GM-CSF, Glucocerebrosidase, Gonadotropin, growth factors, Hedgehog proteins (e.g., Sonic, Indian, Desert), Hemoglobin, Hepatocyte Growth Factor (HGF), Hirudin, Human serum albumin, Insulin, Insulin-like Growth Factor (IGF), interferons (e.g., IFN-α, IFN-β, IFN-γ), interleukins (e.g., IL-1, IL-2, IL-3, IL-4, IL-5, IL-6, IL-7, IL-8, IL-9, IL-10, IL-11, IL-12, etc.), Keratinocyte Growth Factor (KGF), Lactoferrin, leukemia inhibitory factor, Luciferase, Neurturin, Neutrophil inhibitory factor (NIF), oncostatin M, Osteogenic protein, Parathyroid hormone, PD-ECSF, PDGF, peptide hormones (e.g., Human Growth Hormone), Pleiotropin, Protein A, Protein G, Pyrogenic exotoxins A, B, and C, Relaxin, Renin, SCF, Soluble complement receptor I, Soluble I-CAM 1, Soluble interleukin receptors (IL-1, 2, 3, 4, 5, 6, 7, 9, 10, 11, 12, 13, 14, 15), Soluble TNF receptor, Somatomedin, Somatostatin, Somatotropin, Streptokinase, Superantigens, i.e., Staphylococcal enterotoxins (SEA, SEB, SEC1, SEC2, SEC3, SED, SEE), Superoxide dismutase (SOD), Toxic shock syndrome toxin (TSST-1), Thymosin alpha 1, Tissue plasminogen activator, Tumor necrosis factor beta (TNF beta), Tumor necrosis factor receptor (TNFR), Tumor necrosis factor-alpha (TNF alpha), Vascular Endothelial Growth Factor (VEGEF), Urokinase and many others.

One class of proteins that can be made using the compositions and methods for in vivo incorporation of an OMe-L-tyrosine, α-aminocaprylic acid, or photoregulated amino acid such as o-nitrobenzyl cysteine or azobenzyl-Phe described herein includes transcriptional modulators or a portion thereof. Example transcriptional modulators include genes and transcriptional modulator proteins that modulate cell growth, differentiation, regulation, or the like. Transcriptional modulators are found in prokaryotes, viruses, and eukaryotes, including fungi, plants, yeasts, insects, and animals, including mammals, providing a wide range of therapeutic targets. It will be appreciated that expression and transcriptional activators regulate transcription by many mechanisms, e.g., by binding to receptors, stimulating a signal transduction cascade, regulating expression of transcription factors, binding to promoters and enhancers, binding to proteins that bind to promoters and enhancers, unwinding DNA, splicing pre-mRNA, polyadenylating RNA, and degrading RNA.

One class of proteins of the invention (e.g., proteins with one or more OMe-L-tyrosine, α-aminocaprylic acid, or photoregulated amino acid such as o-nitrobenzyl cysteine or azobenzyl-Phe) include expression activators such as cytokines, inflammatory molecules, growth factors, their receptors, and oncogene products, e.g., interleukins (e.g., IL-1, IL-2, IL-8, etc.), interferons, FGF, IGF-I, IGF-II, FGF, PDGF, TNF, TGF-α, TGF-β, EGF, KGF, SCF/c-Kit, CD40LCD40, VLA-4/VCAM-1, ICAM-1/LFA-1, and hyalurin/CD44; signal transduction molecules and corresponding oncogene products, e.g., Mos, Ras, Raf, and Met; and transcriptional activators and suppressors, e.g., p53, Tat, Fos, Myc, Jun, Myb, Rel, and steroid hormone receptors such as those for estrogen, progesterone, testosterone, aldosterone, the LDL receptor ligand and corticosterone.

Enzymes (e.g., industrial enzymes) or portions thereof with at least one OMe-L-tyrosine, α-aminocaprylic acid, or photoregulated amino acid such as o-nitrobenzyl cysteine or azobenzyl-Phe are also provided by the invention. Examples of enzymes include, but are not limited to, e.g., amidases, amino acid racemases, acylases, dehalogenases, dioxygenases, diarylpropane peroxidases, epimerases, epoxide hydrolases, esterases, isomerases, kinases, glucose isomerases, glycosidases, glycosyl transferases, haloperoxidases, monooxygenases (e.g., p450s), lipases, lignin peroxidases, nitrile hydratases, nitrilases, proteases, phosphatases, subtilisins, transaminase, and nucleases.

Many of these proteins are commercially available (See, e.g., the Sigma BioSciences 2003 catalogue and price list), and the corresponding protein sequences and genes and, typically, many variants thereof, are well-known (see, e.g., Genbank). Any of them can be modified by the insertion of one or more photoregulated amino acid (e.g., such as o-nitrobenzyl cysteine or azobenzyl-Phe), O-Me-L-tyrosine, or α-aminocaprylic acid or other unnatural amino acid according to the invention, e.g., to alter the protein with respect to one or more therapeutic, diagnostic or enzymatic properties of interest. Examples of therapeutically relevant properties include serum half-life, shelf half-life, stability, immunogenicity, therapeutic activity, detectability (e.g., by the inclusion of reporter groups (e.g., labels or label binding sites) in the unnatural amino acids, e.g., a photoregulated amino acid (e.g., such as o-nitrobenzyl cysteine or azobenzyl-Phe), O-Me-L-tyrosine, or α-aminocaprylic acid), reduction of LD₅₀ or other side effects, ability to enter the body through the gastric tract (e.g., oral availability), or the like. Examples of diagnostic properties include shelf half-life, stability, diagnostic activity, detectability, or the like. Examples of relevant enzymatic properties include shelf half-life, stability, enzymatic activity, production capability, or the like.

A variety of other proteins can also be modified to include one or more OMe-L-tyrosine, α-aminocaprylic acid, or photoregulated amino acid such as o-nitrobenzyl cysteine or azobenzyl-Phe or other unnatural amino acid of the invention. For example, the invention can include substituting one or more natural amino acids in one or more vaccine proteins with a photoregulated amino acid (e.g., such as o-nitrobenzyl cysteine or azobenzyl-Phe), O-Me-L-tyrosine, or α-aminocaprylic acid, e.g., in proteins from infectious fungi, e.g., Aspergillus, Candida species; bacteria, particularly E. coli, which serves a model for pathogenic bacteria, as well as medically important bacteria such as Staphylococci (e.g., aureus), or Streptococci (e.g., pneumoniae); protozoa such as sporozoa (e.g., Plasmodia), rhizopods (e.g., Entamoeba) and flagellates (Trypanosoma, Leishmania, Trichomonas, Giardia, etc.); viruses such as (+) RNA viruses (examples include Poxviruses e.g., vaccinia; Picornaviruses, e.g. polio; Togaviruses, e.g., rubella; Flaviviruses, e.g., HCV; and Coronaviruses), (−) RNA viruses (e.g., Rhabdoviruses, e.g., VSV; Paramyxoviruses, e.g., RSV; Orthomyxoviruses, e.g., influenza; Bunyaviruses; and Arenaviruses), dsDNA viruses (Reoviruses, for example), RNA to DNA viruses, i.e., Retroviruses, e.g., HIV and HTLV, and certain DNA to RNA viruses such as Hepatitis B.

Agriculturally related proteins such as insect resistance proteins (e.g., the Cry proteins), starch and lipid production enzymes, plant and insect toxins, toxin-resistance proteins, Mycotoxin detoxification proteins, plant growth enzymes (e.g., Ribulose 1,5-Bisphosphate Carboxylase/Oxygenase, “RUBISCO”), lipoxygenase (LOX), and Phosphoenolpyruvate (PEP) carboxylase are also suitable targets for an OMe-L-tyrosine, α-aminocaprylic acid, or photoregulated amino acid such as o-nitrobenzyl cysteine or azobenzyl-Phe or other unnatural amino acid modification.

In certain embodiments, the protein or polypeptide of interest (or portion thereof) in the methods and/or compositions of the invention is encoded by a nucleic acid. Typically, the nucleic acid comprises at least one selector codon, at least two selector codons, at least three selector codons, at least four selector codons, at least five selector codons, at least six selector codons, at least seven selector codons, at least eight selector codons, at least nine selector codons, ten or more selector codons.

Genes coding for proteins or polypeptides of interest can be mutagenized using methods well-known to one of skill in the art and described herein under “Mutagenesis and Other Molecular Biology Techniques” to include, e.g., one or more selector codon for the incorporation of a photoregulated amino acid (e.g., such as o-nitrobenzyl cysteine or azobenzyl-Phe), O-Me-L-tyrosine, or α-aminocaprylic acid. For example, a nucleic acid for a protein of interest is mutagenized to include one or more selector codon, providing for the insertion of the one or more OMe-L-tyrosine, α-aminocaprylic acid, or photoregulated amino acid such as o-nitrobenzyl cysteine or azobenzyl-Phe. The invention includes any such variant, e.g., mutant, versions of any protein, e.g., including at least one photoregulated amino acid (e.g., such as o-nitrobenzyl cysteine or azobenzyl-Phe), O-Me-L-tyrosine, or α-aminocaprylic acid. Similarly, the invention also includes corresponding nucleic acids, i.e., any nucleic acid with one or more selector codon that encodes one or more OMe-L-tyrosine, α-aminocaprylic acid, or photoregulated amino acid such as o-nitrobenzyl cysteine or azobenzyl-Phe.

To make a protein that includes a photoregulated amino acid (e.g., such as o-nitrobenzyl cysteine or azobenzyl-Phe), O-Me-L-tyrosine, or α-aminocaprylic acid, one can use host cells and organisms that are adapted for the in vivo incorporation of the photoregulated amino acid (e.g., such as o-nitrobenzyl cysteine or azobenzyl-Phe), O-Me-L-tyrosine, or α-aminocaprylic acid via orthogonal tRNA/RS pairs. Host cells are genetically engineered (e.g., transformed, transduced or transfected) with one or more vectors that express the orthogonal tRNA, the orthogonal tRNA synthetase, and a vector that encodes the protein to be derivatized. Each of these components can be on the same vector, or each can be on a separate vector, or two components can be on one vector and the third component on a second vector. The vector can be, for example, in the form of a plasmid, a bacterium, a virus, a naked polynucleotide, or a conjugated polynucleotide.

Defining Polypeptides by Immunoreactivity

Because the polypeptides of the invention provide a variety of new polypeptide sequences (e.g., comprising an OMe-L-tyrosine, α-aminocaprylic acid, or a photoregulated amino acid such as o-nitrobenzyl cysteine or azobenzyl-Phe in the case of proteins synthesized in the translation systems herein, or, e.g., in the case of the novel synthetases, novel sequences of standard amino acids), the polypeptides also provide new structural features which can be recognized, e.g., in immunological assays. The generation of antisera, which specifically bind the polypeptides of the invention, as well as the polypeptides which are bound by such antisera, are a feature of the invention. The term “antibody,” as used herein, includes, but is not limited to a polypeptide substantially encoded by an immunoglobulin gene or immunoglobulin genes, or fragments thereof which specifically bind and recognize an analyte (antigen). Examples include polyclonal, monoclonal, chimeric, and single chain antibodies, and the like. Fragments of immunoglobulins, including Fab fragments and fragments produced by an expression library, including phage display, are also included in the term “antibody” as used herein. See, e.g., Paul, Fundamental Immunology, 4th Ed., 1999, Raven Press, New York, for antibody structure and terminology.

In order to produce antisera for use in an immunoassay, one or more of the immunogenic polypeptides is produced and purified as described herein. For example, recombinant protein can be produced in a recombinant cell. An inbred strain of mice (used in this assay because results are more reproducible due to the virtual genetic identity of the mice) is immunized with the immunogenic protein(s) in combination with a standard adjuvant, such as Freund's adjuvant, and a standard mouse immunization protocol (see, e.g., Harlow and Lane (1988) Antibodies, A Laboratory Manual, Cold Spring Harbor Publications, New York, for a standard description of antibody generation, immunoassay formats and conditions that can be used to determine specific immunoreactivity. Additional details on proteins, antibodies, antisera, etc. can be found in U.S. Ser. No. 60/479,931, 60/463,869, and 60/496,548 entitled “Expanding the Eukaryotic Genetic Code”; WO 2002/085923, entitled “IN VIVO INCORPORATION OF UNNATURAL AMINO ACIDS”; patent application entitled “Glycoprotein synthesis” filed Jan. 16, 2003, U.S. Ser. No. 60/441,450; WO 2004/035605, entitled “Glycoprotein Synthesis”; and patent application entitled “Protein Arrays,” attorney docket number P1001US00 filed on Dec. 22, 2002.

### Use of O-tRNA and O-RS and O-tRNA/O-RS Pairs

The compositions of the invention and compositions made by the methods of the invention optionally are in a cell. The O-tRNA/O-RS pairs or individual components of the invention can then be used in a host system's translation machinery, which results in a photoregulated amino acid (e.g., such as o-nitrobenzyl cysteine or azobenzyl-Phe), O-Me-L-tyrosine, or α-aminocaprylic acid being incorporated into a protein. The patent application “In vivo Incorporation of Unnatural Amino Acids” WO 2002/085923 by Schultz, et al. describes processes amenable to use with the current invention and is incorporated herein by reference. For example, when an O-tRNA/O-RS pair is introduced into a host, e.g., Escherichia coli or yeast, the pair leads to the in vivo incorporation of a photoregulated amino acid (e.g., such as o-nitrobenzyl cysteine or azobenzyl-Phe), O-Me-L-tyrosine, or α-aminocaprylic acid, which can be exogenously added to the growth medium, into a protein, e.g., myoglobin or a therapeutic protein, in response to a selector codon, e.g., an amber nonsense codon. Optionally, the compositions of the invention can be in an in vitro translation system, or in an in vivo system(s). Proteins with the photoregulated amino acid (e.g., such as o-nitrobenzyl cysteine and azobenzyl-Phe), O-Me-L-tyrosine, or α-aminocaprylic acid can be used as therapeutic proteins and can be used to facilitate studies on protein structure, interactions with other protein, electron transfer processes in proteins, and the like.

### Kits

Kits are also a feature of the invention. For example, a kit for producing a protein that comprises at least one OMe-L-tyrosine, α-aminocaprylic acid, or photoregulated amino acid such as o-nitrobenzyl cysteine or azobenzyl-Phe in a cell is provided, where the kit includes a container containing a polynucleotide sequence encoding an O-tRNA, and/or an O-tRNA, and/or a polynucleotide sequence encoding an O-RS, and/or an O-RS. In one embodiment, the kit further includes a photoregulated amino acid (e.g., o-nitrobenzyl cysteine or azobenzyl-Phe), O-Me-L-tyrosine, or α-aminocaprylic acid. In another embodiment, the kit further comprises instructional materials for producing the protein. Any composition, system or device of the invention can also be associated with appropriate packaging materials (e.g., containers, etc.) for production in kit form.

### EXAMPLES

The following examples are offered to illustrate, but not to limit the claimed invention. One of skill will recognize a variety of non-critical parameters that may be altered without departing from the scope of the claimed invention.

### Example 1

### Production of Orthogonal Synthetase/tRNA Pair

This example describes the generation of a new orthogonal E. coli tRNA^{Leu}/leucyl tRNA-synthetase (LRS) pair that has been used to selectively incorporate O-Me-L-tyrosine, the C8 amino acid, α-aminocaprylic acid, and the photocaged amino acid, o-nitrobenzyl cysteine, into proteins in yeast in response to the amber nonsense codon, TAG. In addition, it is shown that the latter amino acid, i.e., o-nitrobenzyl cysteine, can be used to photoregulate the activity of the proapoptotic cysteine protease, caspase 3. The development of this and other orthogonal tRNA-synthetase pairs further extends both the nature and number of amino acids that can be selectively introduced into proteins in bacteria, yeast, etc.

Based on the crystal structure of the homologous Thermus thermophilus leucyl tRNA synthetase (ttLRS), see Cusack, et al., EMBO J., 2000, 19:2351-63, it was hoped that the E. coli leucyl synthetase (ecLRS) active site could be evolved to accommodate a wide variety of amino acid side chains. The leucine binding site is a relatively large cavity comprised of side chains and no backbone elements, making possible a large number of mutant active site configurations. In addition, it was hoped that an amber suppressor tRNA-synthetase pair derived from the E. coli leucyl tRNA and cognate synthetase would be orthogonal in yeast, i.e., that this pair would not interact with any of the host tRNAs or synthetases. See, e.g., Soma, et al., Nuc. Acids Res., 1998, 26:4374-81; Soma, et al., J. Mol. Biol., 1996, 263:707-14; and Asahara, et al., J. Mol. Biol. 1993, 231:219-29. This condition would insure that only the unnatural amino acid (but not endogenous amino acids) would be incorporated into proteins at the site specified by the amber codon and no other site.

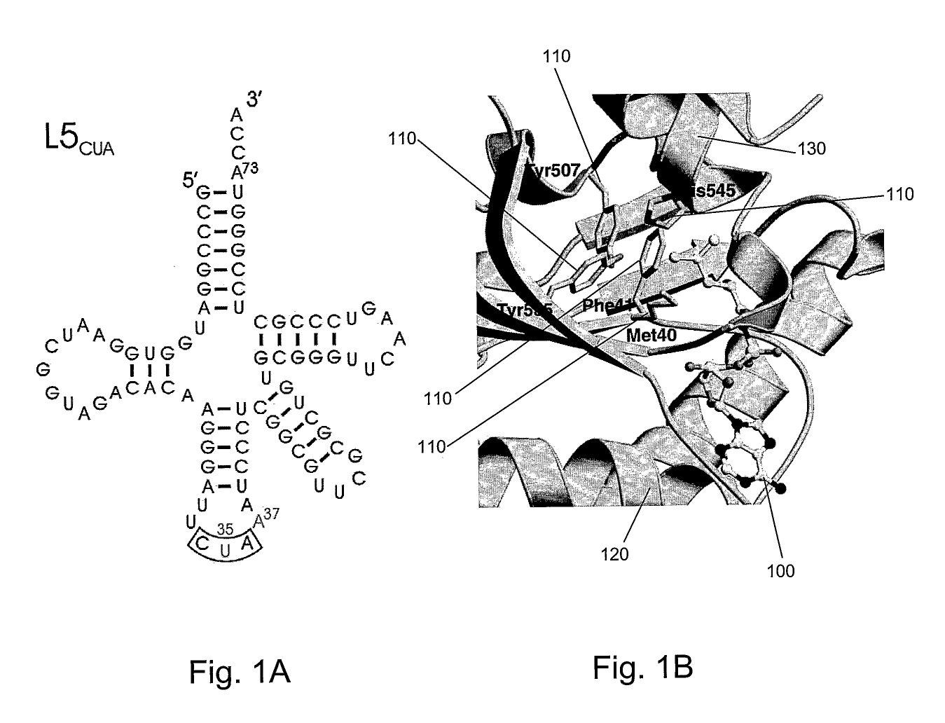

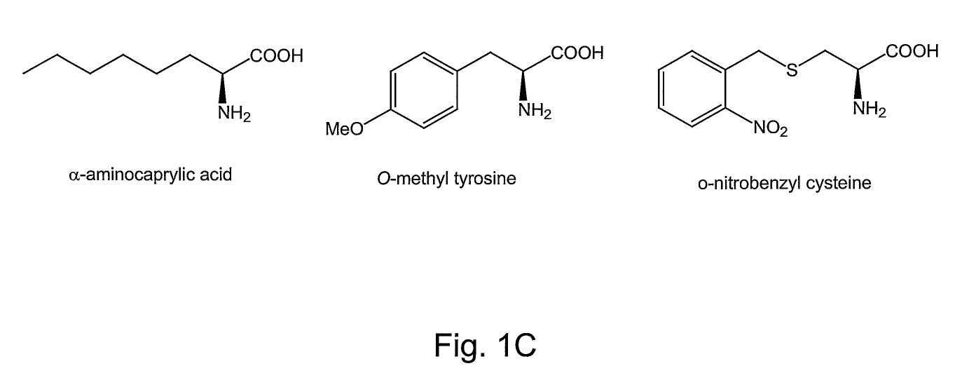

The orthogonality of the E. coli leucyl suppressor tRNA_{CUA} (Leu5_{CUA}) (FIG. 1A), which has U35 and A37 in the anticodon loop, and the corresponding ecLRS was examined in vivo in a selection strain of S. cerevisiae [MaV203:pGADGAL4(2 TAG)]. See Chin, et al., Science, 2003, 301:964-7. This strain harbors the transcriptional activator GAL4 with amber codons at two permissive sites (Thr44 and Arg110). Efficient suppression at both sites leads to expression of three reporter genes: lacZ, his3, and ura3. In the presence of either Leu5_{CUA} or ecLRS alone, no significant β-galactosidase activity was detected above the residual activity in lysates from transformed cells. However, co-expression of both Leu5_{CUA} and ecL-RS resulted in a more than a 10⁴-fold increase in activity, similar to the activity of the previously reported E. coli tRNA^{Try}_{CUA}/ecYRS amber suppressor pair. See ibid. These results indicated that this leucyl pair is orthogonal and can function efficiently to deliver leucine in response to a TAG codon in yeast.

To evolve leucyl synthetases specific for unnatural amino acids, the residues Met40, Leu41, Tyr499, Tyr527, and His537 (the corresponding residues in ttLRS are Met40, Phe41, Tyr507, Tyr535, and His545) were randomized simultaneously to generate an active site library containing ˜10⁷ mutants. See FIG. 1B and Chin, et al., Chem. Biol., 2003, 10:511-9. In FIG. 1B, the ttLRS active site is shown with a bound leucyl sulfamoyl adenylate inhibitor 100 (PDB entry 1H3N). The residues 110 randomized in generating the synthetase library of Example 1 are marked with their amino acid designations. The catalytic domain of LRS is composed of two discontinuous stretches of primary sequences, 120, N-terminal half and 130, C-terminal. These residues form a large hydrophobic pocket surrounding one y-methyl group of the leucine side chain, and are highly conserved in bacterial LRSs. Three unnatural amino acids with distinct electronic and steric properties were selected to probe the adaptability of the LRS active site: O-methyl tyrosine (OmeY, also written as Ome-L-tyrosine herein), α-aminocaprylic acid (C8), and o-nitrobenzyl cysteine (nbC) (see FIG. 1C). The aliphatic side chain of α-aminocaprylic acid should have distinct packing properties relative to the other hydrophobic amino acids, and may allow localization of proteins at membranes. O-nitrobenzyl cysteine can be photocleaved to generate free cysteine on a millisecond timescale, and therefore can be used to photoinitiate a number of biological processes and O-methyl tyrosine can be used to isotopically label proteins selectively (e.g., allow the introduction of NMR and IR probes). Additional libraries with the orthogonal tRNA/leucyl tRNA-synthetase pair can also optionally be created to select for other functional groups with even larger steric bulk especially since this orthogonal pair has a large size in the synthetase active site. Thus, given the tolerating nature of the leucine synthetase, synthetases specific for unnatural amino acids with bulky side chains, e.g., fluorophores such as dansyl and coumarin derivatives can be isolated from such library.

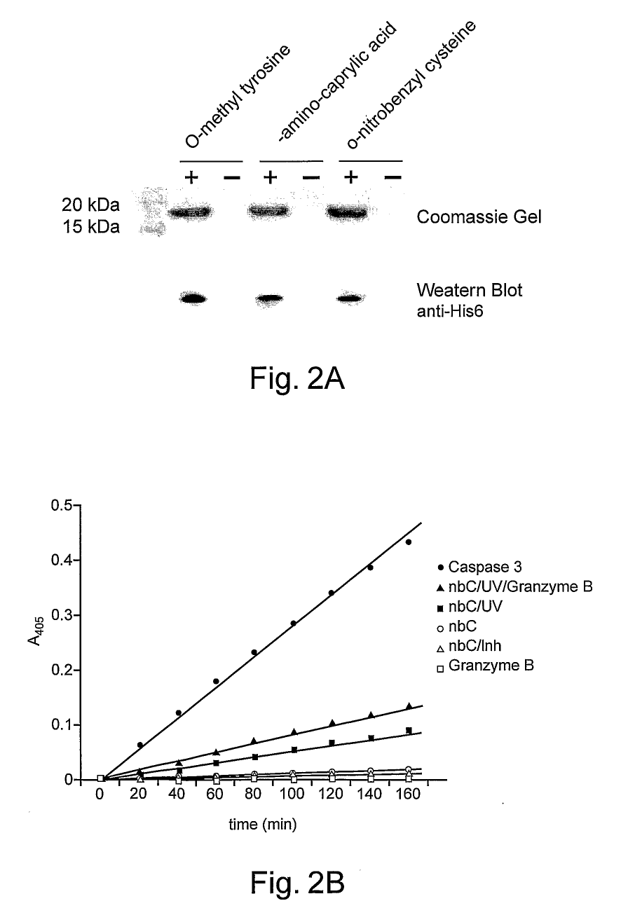

Mutant synthetases specific for each unnatural amino acid were selected based on suppression of the amber codons at position 44 and 110 in the gal4 gene in yeast. Specifically, charging of Leu5_{CUA} by a mutant synthetase with the unnatural amino acid (added to the media at 1 mM concentration) or an endogenous amino acid results in growth either on media lacking histidine (−His) but containing 20 mM 3-aminotriazole (3-AT, a competitive inhibitor of the HIS3 protein), or on media lacking uracil (−ura). In the absence of the unnatural amino acid, cells harboring synthetases that utilize endogenous amino acids show slowed growth on media containing 0.1% 5-fluorootic acid (5-FOA) which is converted into a toxic compound by URA3. See Chin, et al., Chem. Biol. 2003, 10:511-9. Three rounds of alternating positive and negative selections were carried out for each unnatural amino acid. After the final round, clones were chosen for their strict growth dependence on -ura and -his/20 mM 3-AT plates supplemented with 1 mM of the unnatural amino acid. These clones were then tested for suppression of an amber codon at the permissive site 33 in human superoxide dismutase containing a C-terminal hexahistidine tag (hSOD-33TAG-His6) in the presence and absence of the unnatural amino acids in yeast. See Table 2. The best synthetase clones (designated C8RS, OMeYRS, and nbCRS) produced protein only in the presence of their corresponding unnatural amino acid (FIG. 2A and Table 3). FIG. 2A shows expression of hSOD-33TAG-His₆ in the presence (+lanes) and absence (−lanes) of 1 mM unnatural amino acids detected with both Coomassie blue and anti-His6 antibody after Ni-NTA purification. In each case, the yield of the purified protein is comparable to that produced by the wild type orthogonal suppressor tRNA/synthetase pair (˜0.6 mg/L). In addition, electrospray-ionization ion-trap mass spectrometry revealed that the total mass of the intact hSOD protein was consistent with site specific incorporation of the unnatural amino acid in response to the amber codon (Table 3). These results indicate that all three amino acids are incorporated into proteins with high efficiency and very high fidelity.

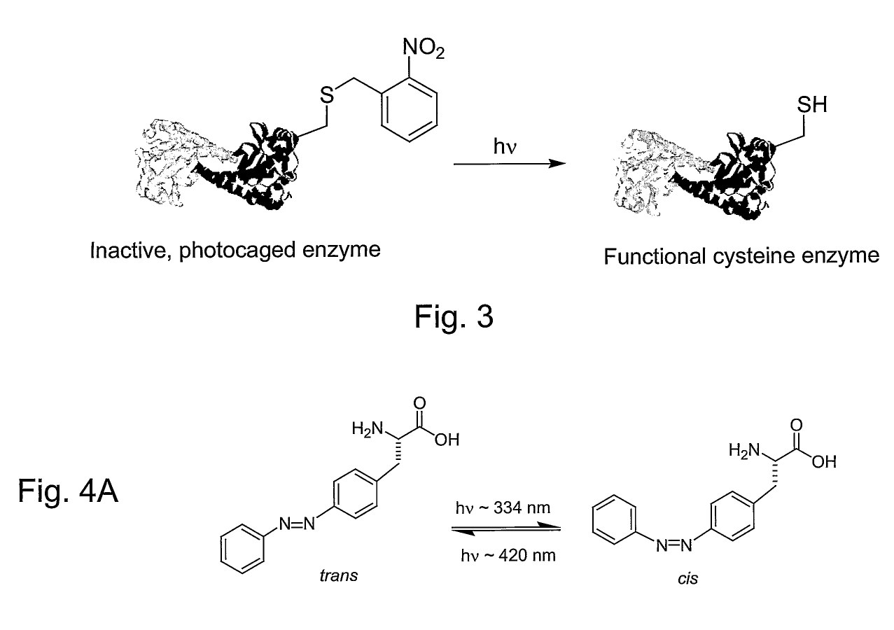

The efficient and selective biosynthetic incorporation of o-nitrobenzyl cysteine into proteins should allow both temporal and spatial photoregulation of proteins with essential cysteine residues in vitro and in vivo. See, e.g., Self, et al., Nat. Med., 1996, 2:817-20; Philipson, et al., Am. J. Physiol. Cell Physiol., 2001, 281:C195-206; and Pollitt, Agnew. Chem. Int. Ed. Engl., 1998, 37:2104-7. UV irradiation of the nbC-33-hSOD mutant with an UV lamp for 20 minutes resulted in the cleavage of the benzylic C—S bond and subsequent release of the free thiol group and o-nitrobenzaldehyde. A 70% conversion to the decaged hSOD was detected by RPLC and full-length mass spectral analysis. The observed mass of 16584.2 Da for hSOD-33Cys is in agreement with the calculated mass of 16585.2 Da. NbC-33-hSOD was the only other observed protein. This is in agreement with previous studies of the phodeprotection of Boc-nbC. See, e.g., Smith, et al., Org. Lett., 2002, 4:4041-4; and, FIG. 3 which schematically illustrates photoregulation of o-nitrobenzyl cysteine.

To further illustrate the utility of o-nitrobenzyl cysteine, the active site cysteine of the human proapoptotic protein caspase 3 was substituted with o-nitrobenzyl cysteine. Like other caspases involved in apoptosis, this enzyme exists in the cytosol as an inactive zymogen. A two-chain mature enzyme is generated after precise cleavage at internal aspartate residues, either by activated caspase 3 or by other proteases such as the serine protease granzyme B. See Trapani, Genome Biol., 2001, 2(12), REVIEWS3014.1-3014.7. To photocage caspase 3 activity, the codon for the catalytic Cys163 residue was mutated to TAG and the protein was expressed under an inducible galactose promoter in the presence of 1 mM o-nitrobenzyl cysteine, nbCRS, and Leu5_{CUA}. Caspase 3 activity was measured in cell lysates after 10 minutes of irradiation with or without further incubation with granzyme B. Only after photolysis was there any detectable protease activity in the cell lysate (FIG. 2B), both in the presence and in the absence of Granzyme B. Under these conditions, approximately 40% of the caged caspase was converted to the active enzyme. Moreover, expression of wildtype caspase 3 is toxic to yeast, whereas expression of the nbC163 caspase mutant had no effect on growth rates. FIG. 2B shows measurement of caspase 3 activity with a 7-amino-4-trifluoromethyl coumarin substrate (absorption at 405 nm) in an untreated cell lysate (nbC), after irradiation (nbC/UV), after irradiation in presence of granzyme B (nbC/UV/granzyme B), and in the presence of a caspase 3 inhibitor (nbC/Inh). Commercial recombinant caspase 3 was used as a positive control and granzyme B as a negative control.

Protocol, Materials and Methods:

### Evolved Synthetases:

TABLE 2
Synthetase clones in this example isolated from
the library for various unnatural amino acids.

| Wild type | Met40 | Leu41 | Tyr499 | Tyr527 | His537 |
|---|---|---|---|---|---|
| α-aminocaprylic acid | | | | | |
| 1D7 | Ala | Ala | Pro | Val | Gly |
| 1G8 | Val | Met | Leu | Leu | Gly |
| 2F2 | His | Pro | Ala | Met | Gly |
| 2F5 | Val | Tyr | Leu | Leu | Gly |
| O-methyl tyrosine | | | | | |
| 3A7 | Leu | Glu | Arg | Ala | Gly |
| 3A2 | Met | Glu | Arg | Phe | Gly |
| 3F11 | Leu | Glu | Arg | Cys | Gly |
| 3E7 | Phe | Glu | Arg | Thr | Gly |
| o-nitrobenzyl cysteine | | | | | |
| 1A3 | Gly | Glu | Arg | Leu | Gly |
| 3A12 | Gly | Trp | Ala | Leu | Gly |
| 3H11 | Trp | Ser | Ile | Ala | Gly |
| 4E1 | Gly | Thr | Trp | Leu | Gly |

TABLE 3
Active site residues of the best synthetases evolved in this example
and mass of intact hSOD produced using these synthetases in the
presence of their respective unnatural amino acids.

| Residue # | 40 | 41 | 499 | 527 | 537 | Calculated Mass | Observed Mass |
|---|---|---|---|---|---|---|---|
| ecLRS | Met | Leu | Tyr | Tyr | His | 16594.3 Da | 16594.2 Da |
| C8RS | Val | Met | Leu | Leu | Gly | 16622.3 Da | 16622.3 Da |
| OMeYRS | Leu | Glu | Arg | Ala | Gly | 16658.3 Da | 16658.2 Da |
| nbCRS | Trp | Ser | Ile | Ala | Gly | 16720.3 Da | 16720.1 Da |

Unless otherwise stated, all polymerase chain reactions (PCR) in this example were performed using the Expand PCR kit from Roche (Nutley, N.J.) according to the manufacturer's instructions. All yeast transformations, including the ecLRS library, were performed using YEASTMAKER Yeast Transformation system 2 from Clontech (Mt. View, Calif.) according to the manufacturer's specifications.

Library construction and selection: An amber suppressor tRNA Leu5_{CUA}, based on tRNA^{Leu}_{UUA} (accession number K00225), see Yamaizumi, et al., 1980, J. Biol. Chem., 255(5):2220-5, was constructed along with flanking sequences by overlap PCR with four primers:

Ect1 -
GATCACCGGTAAGCTTCCCGATAAGGGAGCAGGCCAGTAAAAAGCATTAC
CCCGTGCC
Ect2 -
GGATTTAGAATCCCTTGTGTCTACCGATTCCACCATCCGGGCACGGGGTA
ATGC
Ect3 -
AGGGATTCTAAATCCCTCGGCGTTCGCGCTGTGCGGGTTCAAGTCCCGCT
CCGG
Ect4 -
TTAGGCTAGCGGGAAGTTCAGGGACTTTTGAAAAAAATGGTACCCGGAGC
GGGACThTG

The amplified DNA was digested with restriction enzymes AgeI and NheI and inserted into pA5/tRNACUA (see Chin, et al., Science 2003, 301, 964-7 and Chin, et al., Chem Biol 2003, 10, 511-9) at the corresponding sites to generate plasmid pA5/L5_{CUA}. LeuRS was amplified from E. coli genomic DNA (primers ECF-GCGC GAATTCAGTATGGAAGAGCAATACCGCCCGGAAGAG and ECR-GCGCGCGGCCGCTTAGCCAACGACCAGATTGAGGAG), digested with EcoRI and NotI, and ligated into the corresponding sites of the plasmid pA5/L5_{CUA} to yield plasmid pECLRS/L5_{CUA}. A LeuRS library with 5 randomized residues (Met40, Leu41, Tyr499, Tyr527, and His537) was constructed using enzymatic inverse polymerase chain reaction (EIPCR) (see Stemmer, et al., 1992, Biotechniques, 13(2):214-20) in a similar fashion to the construction of the ecTyrRS library (see Chin above). All constructs and the diversity of the library were confirmed by DNA sequencing.

β-Galactosidase assay: Rapid and sensitive detection of β-galactosidase activity in cell lysates was performed with the Galacto-Star™ chemiluminescent system from Applied Biosystems (Bedford, Mass.) according to the manufacturer's instructions. This assay has a dynamic range of 2 fg to 20 ng of purified enzyme. Cells from a 15 ml culture (OD₆₀₀<2) were lysed by freeze-thaw cycles. Chemiluminescence was measured with Analyst™ AD 96.384 (LJL Biosystems, Sunnyvale, Calif.). Standard deviation was obtained from five parallel measurements. The relative activity between samples was normalized with total protein concentration in the lysate.

Synthesis of o-nitrobenzyl cysteine: This unnatural amino acid was synthesized according to a known procedure (see Smith, et al., Organic Lett. 2002, 4, 4041-44). The other unnatural amino acids where obtained from Sigma-Aldrich (Milwaukee, Wis.).

Expression and analysis of protein containing unnatural amino acids: The hSOD proteins were expressed and purified as described previously (see Chin above). For Western analysis, proteins were detected with anti-His6 (C-term) antibody (R930-25, Invitrogen, Carlsbad, Calif. and sc-2308, Santa Cruz Biotech, Santa Cruz, Calif.) and visualized with ECF (Amersham Biosciences, Piscataway, N.J.)

Mass Spectrometry Measurements: The purity and primary structure of the intact desalted proteins were assessed by electrospray-ionization ion trap mass spectrometry (Bruker 3000). All experimentally determined masses have standard deviation less than 1 amu.

Caspase 3 assay: The wild type human caspase 3 gene was donated by Jennifer Harris. The codon for Cys163 was mutated to TAG with overlap PCR, and the mutant gene was inserted into pYES2 yeast expression vector (Invitrogen) at the BamHI and XhoI sites to yield pYES2-caspase3TAG. This plasmid was cotransformed with pEcpC7/t1 into S. cerevisiae strain InvSc (Invitrogen). For protein expression, a saturated cell culture grown in media SD+raffinose−Trp-ura (Qbiogene, Carlsbad, Calif.) was used to inoculate SD+raffinose+galactose−Trp-ura (Clontech) supplemented with 1 mM nbC to an OD₆₀₀ of 0.4. Cells were pelleted after 7-8 hours induction, washed and resuspended in PBS, and lysed with freeze and thaw cycles. After pelleting the cell debris, lysate was cleared using a 0.22 μm filter. The lysate was then photolysed with a handheld UV lamp (Model ENF-240C, Spectronics Corporation, Lincoln, Nebr.) at long wavelength (>365 nm) for 10 minutes. Some of the treated lysate was then incubated with 100 U of granzyme B (Biomol, SE-238, Plymouth Meeting, Pa.) at 30° C. for 30 minutes. These lysates, together with the untreated samples, were used in caspase 3 assay with CASPASE-3 Cellular Activity Assay Kit PLUS (Biomol, Plymouth Meeting, Pa.) according to the manufacturer's instructions. The reaction was monitored continuously on Spectra Max 190 (Molecular Devices, Sunnyvale, Calif.) at 405 nm.

This example shows that a new orthogonal pair has been generated in yeast and three structurally distinct unnatural amino acids have been efficiently incorporated into proteins using this new orthogonal pair, including a photocaged cysteine. It should be possible to evolve LRS mutants to incorporate additional unnatural amino acids including caged amino acids that can be deprotected at longer wavelengths in vivo (e.g., nitroveratyrl Cys, Tyr, or Ser), as well as fluorophores and spin-labeled amino acids.

### Example 2

### The Incorporation of a Photoisomerizable Amino Acid into Protein in E. coli

This example shows the generation of an orthogonal tRNA-aminoacyl tRNA synthetase pair that allows the selective incorporation of the photoisomerizable amino acid phenylalanine-4′-azobenzene (also termed azobenzyl-Phe) into proteins in E. coli in response to the amber codon TAG. Furthermore, the example shows that azobenzyl-Phe can be used to photomodulate the binding affinity of an E. coli transcription factor to its promoter. It will be appreciated that “azobenzyl-Phe” is used to designate the photoisomerizable amino acid phenylalanine-4′-azobenzene herein (both in this example and throughout the specification). Other equivalent terms, or alternative nomenclatures, which can used herein include, e.g., 4-azobenzene-L-phenylalanine, p-azobenzene-L-phenylalanine, L-phenylalanine-4′-azobenzene, and p-azobenzyl-L-phenylalanine.

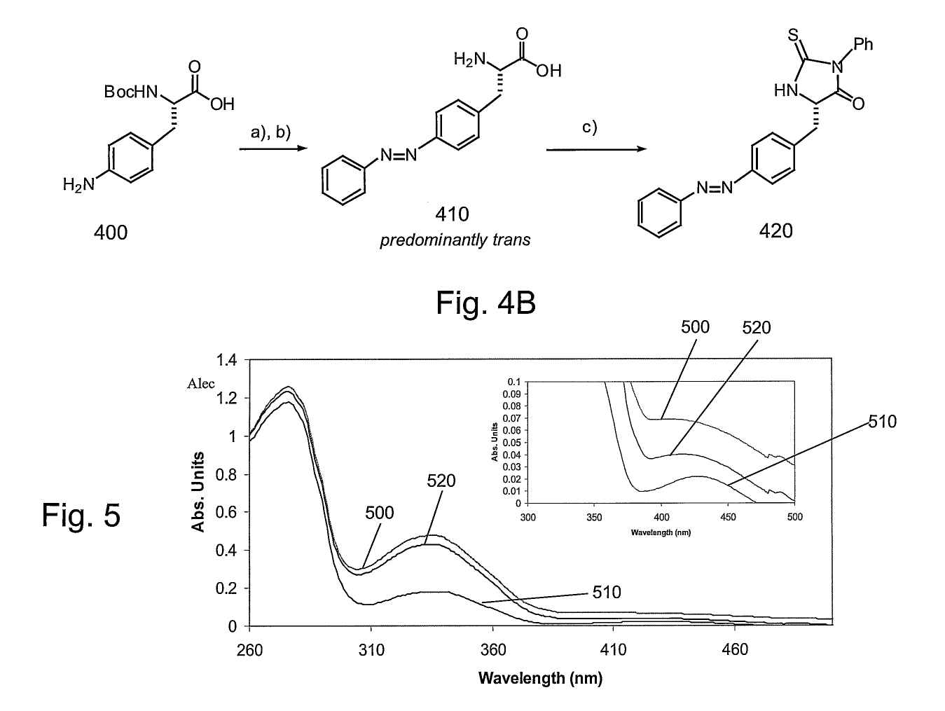

Azobenzene can undergo a reversible cis-trans photochemical isomerization. Irradiation at 320-340 nm converts the thermodynamically more stable trans isomer to the cis isomer; the cis form reverts thermally or upon irradiation at >420 nm. See, e.g., Zimmerman, et al., J. Am. Chem. Soc., 1958, 80:3528-31, and Rau, H. In Photochromism: molecules and systems, Duerr, H. Bouas.-Laurent., H. Eds. Elsevier Science B.V., Amsterdam, Neth: 1990, pp 165-92. See also FIG. 4A. Because the two isomers differ significantly in geometry and absorption maxima, placement of an azobenzene moiety in close proximity to a substrate or ligand binding site in an enzyme, receptor or ion channel allows one to reversibly modulate the binding affinity, and consequently, the activity of a protein. To biosynthetically incorporate the azobenzene moiety into proteins, the unnatural amino acid azobenzyl-Phe was used. See FIG. 4A. Azobenzyl-Phe was synthesized by coupling of nitrobenzene to N-Boc-p-aminophenylalanine followed by Boc deprotection. See FIG. 4B, which shows the synthesis of azobenzyl-Phe (the center structure, 410, also shown in FIG. 4A) and azobenzyl-Phe-PTH (structure, 420). The synthesis steps in FIG. 4B include: PhNO, glacial acetic acid, room temperature for (a); 1, 4N HCl, dioxane, 0° C. for (b); and, PhNCS, pyridine-H₂O, 40° C. followed by 1N HCl at 80° C. for (c). The short distance between the azobenzene moiety and C_{α} carbon of the structure in FIG. 4A minimizes the number of conformational isomers of the side chain, which is thought to result in a greater differential effect of cis-trans isomerization on the active site structure.

To selectively incorporate azobenzyl-Phe at defined sites in proteins in E. coli, an orthogonal tRNA-aminoacyl t-RNA synthetase pair was evolved that uniquely specifies the structure in FIG. 4A in response to the amber TAG codon. Previously, it has been reported that the M. jannaschii tyrosyl-tRNA synthetase (MjTyrRS) and a mutant tyrosyl amber suppressor tRNA (MjtRNA^{Tyr}_{CUA}) function efficiently in protein translation in E. coli, but do not cross react with endogenous tRNAs or synthetases. See Wang, et al., J. Am. Chem. So., 2000, 122:5010-11. To alter the specificity of the MjTyrRS synthetase to selectively recognize azobenzyl-Phe (and not an endogenous amino acid), a library of 109 TyrRS mutants was generated by randomizing six residues (Tyr-32, Leu-65, Phe-108, Gln-109, Asp-158 and Leu-162) in the tyrosine binding site of TyrRS. The x-ray crystal structures (see Kobayashi, et al., Nat. Struct. Biol. 2003, 10:425-32 and Zhang, et al., Protein Sci. 2005, 14:1340-9) of the M. jannaschii TyrRS show that these residues are all close to the aryl ring of bound tyrosine (and include Tyr-32 and Asp-158 which form hydrogen bonds with the hydroxyl group of tyrosine). Both positive and negative selections were then applied to the MjTyrRS library (pBK-lib2). See Santoro, et al., Nature Biotechnology 2002, 20:1044-8 and Wang, et al., Science 2001, 292:498-500. In the positive selection, cell survival was dependent on the suppression of an amber codon introduced at a permissive site in the chloramphenicol acyl transferase gene when cells cotransformed with pBK-lib2 and tRNA^{TYR} were grown in the presence of 1 mM azobenzyl-Phe and chloramphenicol. Synthetase clones from surviving cells were then transformed into cells containing the orthogonal tRNA and a gene encoding the toxic barnase protein with amber mutations introduced at three permissive sites. These cells were grown in the absence of azobenzyl-Phe to remove any clones that utilized endogenous amino acids.

After five rounds of selection (three positive and two negative) 10 synthetase clones were identified that allowed cells to survive up to 120 ug/mL chloramphenicol in the presence of azobenzyl-Phe and up to 20 ug/mL chloramphenicol in the absence of the unnatural amino acid. All ten synthetase clones had the same mutations: Tyr32Gly, Leu65Glu, Phe108Ala, Gln109Glu, Asp158Gly and Leu162His. Based on the wild-type synthetase structure, these mutations were thought to create an enlarged tyrosine binding pocket to accommodate the azobenzene moiety. For example, the bulky amino acid side chains of Tyr32 and Phe108 of the wild-type synthetase were replaced by glycine or alanine residues in azobenzyl-PheRS. In addition, the side chains of both Tyr32 and Asp158, which are involved in hydrogen bonding to the phenolic hydroxyl of the bound tyrosine, were substituted by glycine.

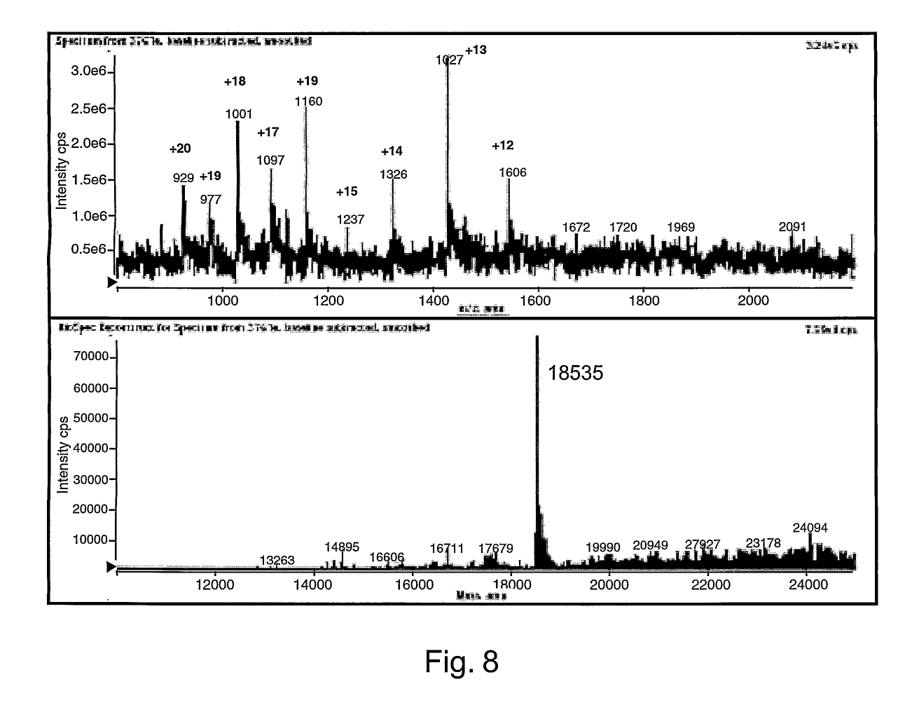

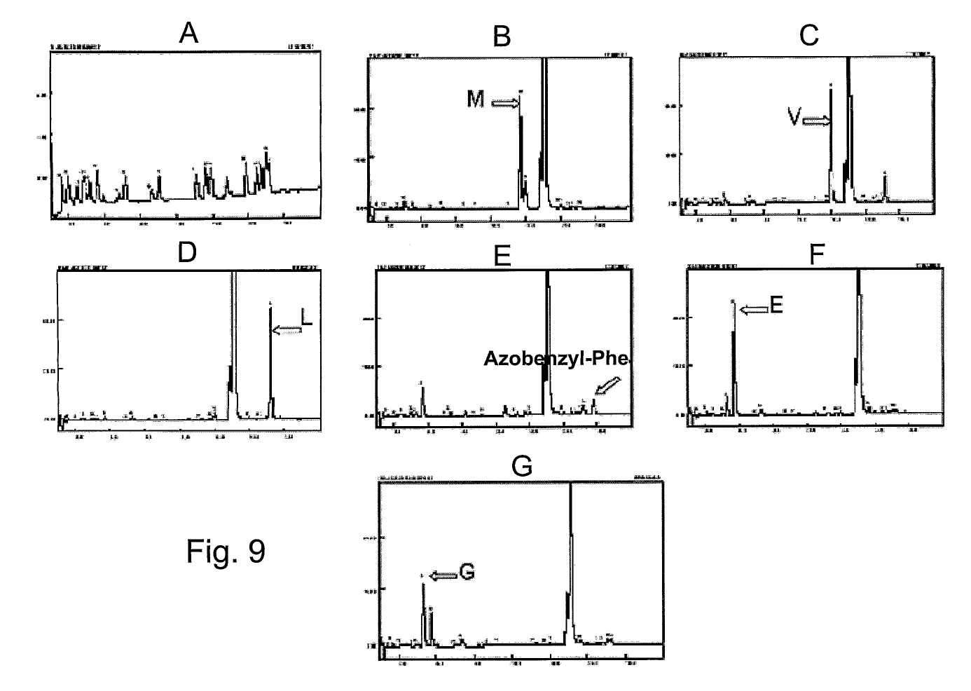

To determine the fidelity and the efficiency of incorporation of azobenzyl-Phe into proteins, an amber codon was substituted for residue 75 in sperm whale myoglobin containing a C-terminal His6 tag. Protein expression was carried out in the presence of the selected synthetase (azobenzyl-PheRS) and MjtRNA^{Tyr}_{CUA} in E. coli grown in minimal media supplemented with 1 mM of azobenzyl-Phe. As a negative control, protein expression was simultaneously carried out in absence of azobenzyl-Phe. Analysis of the purified protein by SDS-PAGE and Western blot showed protein was expressed only in the presence of the structure in FIG. 4A. FIG. 7 shows the characterization of the genetically encoded unnatural amino acid azobenzyl-Phe containing mutant proteins. Expression of mutant myoglobins (Myo4TAG and Myo75TAG, are indicated by arrow 700) and mutant CAP (CAP125TAG, indicated by arrow 710) in the presence (+UAA) and absence (−UAA) of 1 mM unnatural amino acid (UAA=azobenzyl-Phe), detected with both Gelcode Blue® and anti-His6 antibody after Ni-NTA purification. Both ESI (electrospray ionization) and MALDI-TOF (matrix-assisted laser desorption-time of flight) mass spectrometric analyses of the mutant myoglobin gave an observed average mass of 18534 Da, close to the theoretical average mass of 18535.33 Da. No other peaks resulting from incorporation of any common amino acid into myoglobin-75TAG were observed in the mass spectra. See FIG. 8. In addition, mutant myoglobin having azobenzyl-Phe substituted for amino acid 4, was purified and subjected to N-terminal protein sequencing. Edman degradation showed that the fourth position was occupied by only a nonstandard amino acid (and none of the common 20 amino acids), whose elution time matched that of the independently synthesized phenylthiohydantoin derivative of azobenzyl-Phe. See FIG. 9.

To further characterize the photochemical properties of proteins containing azobenzyl-Phe, this amino acid was introduced into the E. coli catabolite activator protein, (CAP). CAP is a homodimeric bacterial transcriptional activator that regulates a number of catabolite sensitive operons in E. coli. See, e.g., Busby, et al., J. Mol. Biol. 1999, 293:199-213; Lawson, et al., Curr. Opin. Struct. Biol., 2004, 1:10-20; and, Schultz, et al., Science 1991, 253:1001-7. Binding of cAMP to CAP results in conformational changes in the protein that increase its binding affinity to its promoter, resulting in enhanced transcription from CAP-dependent promoters. An amber codon was introduced for Val125, a residue in the dimerization interface. A C-terminal His₆ tag was added, and this mutant was expressed in rich media in the presence of 1 mM azobenzyl-Phe, azobenzyl-PheRS and Mj tRNA^{Tyr}_{CUA}. Upon Ni-NTA purification, about 1.5 mg of mutant CAP was obtained per liter of cultured cells, compared to about 3 mg/L for wild-type CAP. A UV-visible spectrum (FIG. 5) of this mutant protein shows a distinct absorbance peak at 334 nm corresponding to the trans-azobenzene chromophore in FIG. 4A. Irradiation of the mutant CAP at 25° C. with 334 nm light led to a decrease in the 334 peak and an increased absorbance at 420 nm, consistent with a trans to cis isomerization to afford a photostationary state of approximately 45% trans, 55% cis azobenzene. FIG. 5. The isomerized mutant was then irradiated with 420 nm light resulting in complete conversion back to the 334 nm band. FIG. 5, line 500, shows absorption spectra of the mutant CAP (CAP125TAG; 50 uM) in 50 mM sodium phosphate, 300 mM NaCl, 250 mM imidazole, pH 8.0 buffer. Line 510, shows absorption after Ni-NTA purification, prior to any photoirradiation. Line 520, shows absorption after photoirradiation at 334 nm, 40 minutes. Reversibly switched back by 420 nm light, 40 minutes. The inset in FIG. 5 is an enlarged view in the range >400 nm. These results show that azobenzyl-Phe can be selectively incorporated into proteins with high fidelity and undergo reversible cis-trans isomerization upon irradiation with light of appropriate wavelengths.

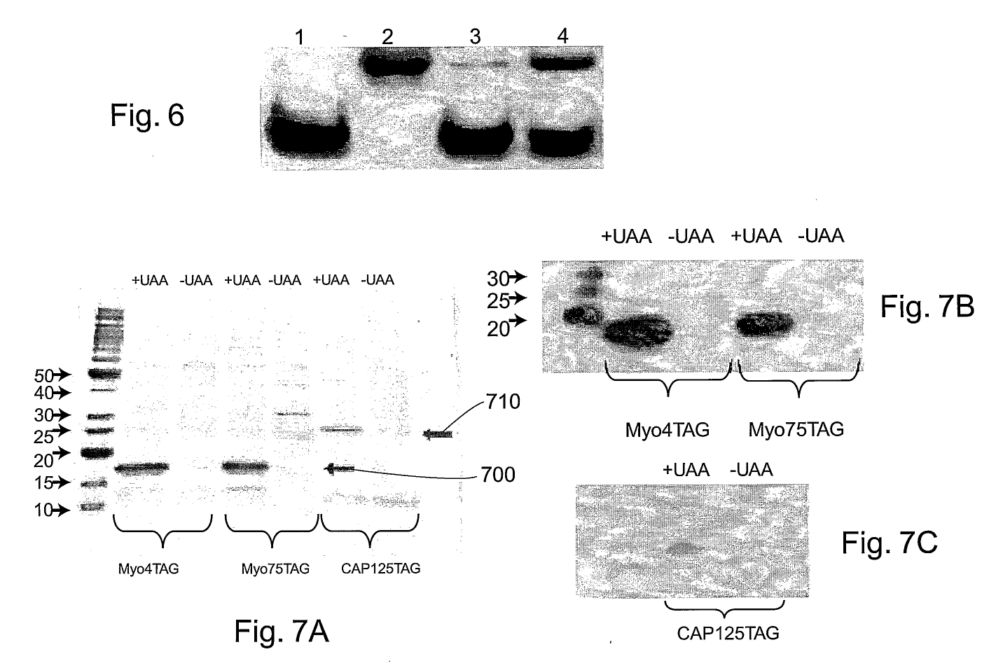

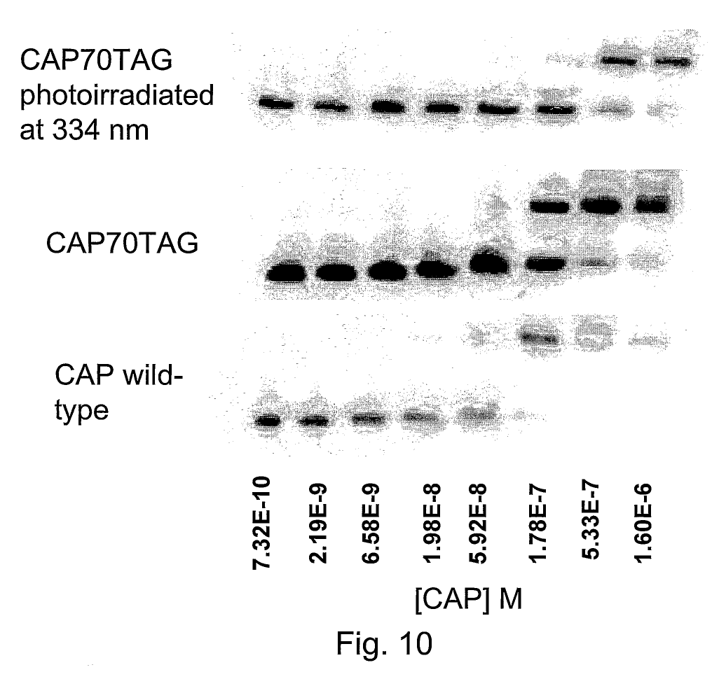

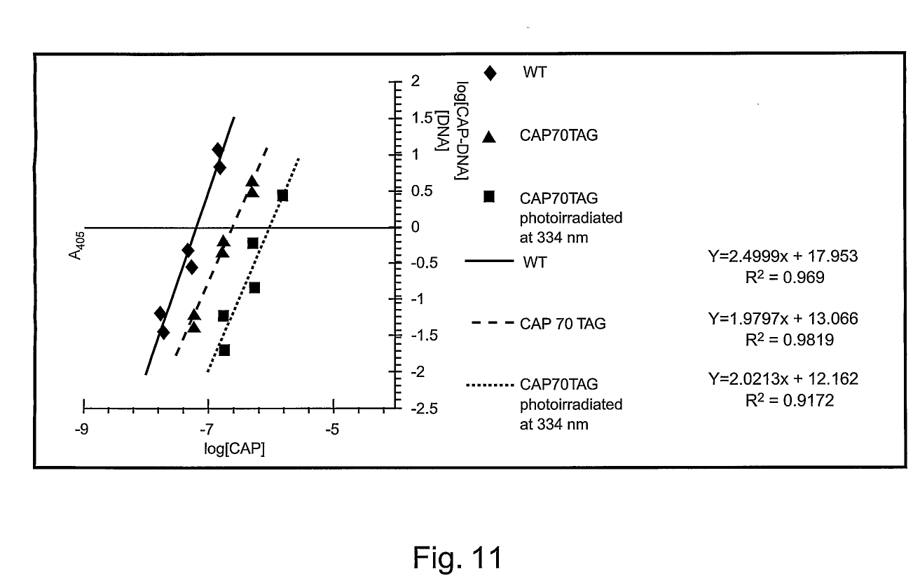

To determine whether azobenzyl-Phe could be used to photoregulate the DNA binding affinity of CAP, azobenzyl-Phe was substituted for Ile70. The crystal structure of the CAP-cAMP-DNA complex (see, e.g., Weber, et al., J. Mol. Biol. 1987, 198:311-26; and, McKay, et al., J. Biol. Chem. 1982, 257:9518-24) shows that Ile70 lies in close proximity to residues Glu72, Arg82, Thr27 and Ser128, which form an intricate network of interactions with cAMP (see Fried, et al., J. Mol. Biol. 1984, 172:241-62). Thus the trans and cis isomers might differentially affect the binding affinity of cAMP to CAP, and as a consequence, the affinity of CAP for its promoter. The mutant protein was expressed and purified on Ni-NTA followed by FPLC purification with a mono-S column with a gradient of 25 mM NaCl to 1 mM NaCl over twenty minutes. FIG. 10. The binding constant (K_{b}) of the mutant CAP protein to a purified lac promoter containing the primary CAP binding site was then determined, both before and after irradiation at 334 nm (FIG. 6). FIG. 6 shows a gel-mobility shift assay to determine CAP (wild-type or mutant CAP70TAG; 160 nM) binding to the lactose promoter fragment (33 nM) in buffer containing 20 uM cAMP. Lane 1 is DNA only. Lane 2 is DNA and CAP wild type, while Lane 3 is DNA and CAP70TAG (photoirradiated at 334 nm). Lane 4 is DNA and CAP70TAG prior to photoirradiation at 334 nm. Using the serial-dilution technique and gel shift assays, a 4-fold lower K_{b} was observed for the trans CAP70azobenzyl-Phe mutant (K_{b}˜40×10⁶M⁻¹) compared to wt CAP (K_{b}˜1.6×10⁷ M⁻¹) in the presence of cAMP (20 μM). Following photoirradiation at 334 nm, the K_{b} of the mutant CAP decreased fourfold to 1.0×10⁶M⁻¹. This is consistent with a photostationary state of 50% cis in which 75% of the homodimers have at least one subunit in the cis form which has a significantly reduced affinity for cAMP. The isomerized CAP70azobenzyl-Phe sample was then switched back to a predominantly trans state using light >420 nm and allowed to incubate at 4° C. Analysis by gel-shift assays of this sample, resulted in complete recovery of the trans form of CAP with a K_{b} of 4.0×10⁶ M⁻¹. FIG. 11 shows the EMSA method of determination of CAP binding affinity for primary lac binding site. For each CAP protein concentration in which the DNA was partially bound, the intensity of both the free DNA and protein-DNA band was measured using a densitometer. Concentrations of free CAP, free DNA and DNA-bound CAP were determined. The log [CAP] was then plotted against log([CAP bound DNA]/[Free DNA]). The binding constant K_{b} was determined using the interpolated concentration of CAP at the x-intercept. These results show that azobenzyl-Phe can be used to photoregulate the binding affinity of a transcription factor to its promoter sequence. Although complete conversion of trans azobenzyl-Phe to the cis form cannot be obtained due to their overlapping absorption spectra, these results strongly suggest that the genetic incorporation of azobenzyl-Phe into proteins should be useful for temporally regulating a variety of biological processes in vitro and in living cells.

Protocols, Materials, and Methods

All chemicals in this example were obtained from commercial sources and used without further purification. ¹H NMR spectra were recorded on a Bruker AMX-400 instrument with chemical shifts reported relative to either residual CHCl₃ (7.25 ppm), residual HOD (4.80 ppm), or residual CH₃OD (3.30 ppm). Mass spectra were acquired at the Scripps Center for Mass Spectrometry (La Jolla, Calif.) and Mass Spectrometry Center, Stanford University (Palo Alto, Calif.).

### Synthesis of N-(tert-Butoxycarbonyl)-L-phenylalanine-4′-azobenzene

L-N-tert-Butoxycarbonyl-p-aminophenylalanine (1 g, 3.6 mmol) was dissolved in glacial acetic acid (200 mL) at room temperature. To this solution nitrobenzene (578 mg, 5.4 mmol) was added in one portion and the mixture was allowed to stir for 8 hours. The reaction mixture was quenched with satd. NaHCO₃ solution (300 mL) and extracted with ethyl acetate (3×100 mL). The organic layers were then combined, dried (anhydrous MgSO₄) and concentrated on a rotary evaporator. The crude material was then purified via silica gel column chromatography (CH₂Cl₂-MeOH. 90:10). Thus, the final product was obtained as an orange solid (900 mg, 68%); ¹H NMR (400 MHz, CD₃OD) δ 1.30 (s, 9H), 2.93 (dd, J=9, 14 Hz, 1H), 3.19 (dd, J=5, 14 Hz, 1H), 4.33 (dd, J=5, 9 Hz, 1H), 7.35 (d, J=8 Hz, 2H), 7.44 (m, 3H), 7.77 (d, J=8 Hz, 2H), 7.82 (d, J=8 Hz, 2H); HRMS (ESI) calcd for C₂₀H₂₃N₃O₄Na (M+Na⁺) 392.1586, obsd. 392.1578.

### Synthesis of L-Phenylalanine-4′-azobenzene

To a solution of N-(tert-Butoxycarbonyl)-L-phenylalanine-4′-azobenzene (azobenzyl-Phe as in FIG. 4A) (546 mg, 1.5 mmol) in dioxane (6 mL) at 0° C. was added 4N HCl (2 mL) and the mixture was allowed to stir for 3 hours. It was then concentrated on a rotary evaporator and thoroughly dried under vacuum (Δ, P₂O₅ sidearm) to provide the final product as an orange solid (380 mg, 94%); ¹H NMR (400 MHz, D₂O) δ 2.46 (dd, J=1, 10 Hz, 1H), 2.83 (dd, J=1, 12 Hz, 1H), 3.23 (dd, J=10, 12 Hz, 1H), 7.00 (m, 5H), 7.32 (d, J=6 Hz, 2H), 7.38 (d, J=6 Hz, 2H); HRMS (ESI) calcd for C₁₅H₁₄N₃O₂ (M-H)⁻ 268.1092, obsd. 268.1083.

### Synthesis of Phenylthiohydantoin-phenylalanine-4′-azobenzene (PTH-azobenzyl-Phe)

The amino acid L-Phenylalanine-4′-azobenzene (azobenzyl-Phe) (200 mg, 0.74 mmol) was dissolved in pyridine-water (1:1; 5 mL) and the pH was adjusted to 8.6 using 2N NaOH. The mixture was stirred at 40° C. and phenylthiocyanate (200 uL, 1.48 mmol) was added dropwise. After 1 hour, the mixture was extracted with toluene (3×5 mL), the aqueous layer was cooled over ice and acidified to pH˜1 via the addition of 2N HCl. The resultant precipitate was filtered and dried (in vacuo) to provide phenylthiocarbamoyl-L-phenylalanine-4′-azobenzene (PTC-azobenzyl-Phe) (239 mg crude). The PTC-azobenzyl-Phe was then dissolved in 1N HCl (2.5 mL) and the solution was heated at 80° C. for 10 minutes and then cooled to ambient temperature. The reaction mixture was then extracted with ethyl acetate (3×5 mL), the organic layers combined and dried (MgSO₄). Flash chromatography (CH₂Cl₂-MeOH. 90:10) of the crude provided the title compound (50 mg, 18%); ¹H NMR (400 MHz, CDCl₃) δ 3.18 (dd, J=7, 14 Hz, 1H), 3.45 (dd, J=4, 14 Hz, 1H), 4.60 (dd, J=4, 7 Hz, 1H), 7.12 (app d, J=6 Hz, 1H), 7.25 (m, 2H), 7.40-7.55 (m, 7H), 7.93 (app d, J=8 Hz, 4H); HRMS (ESI) calcd for C₂₂H₁₈N₄OSNa (M+Na+) 409.1099, obsd. 409.1106.

Plasmids and Cell Lines

The following plasmids and cells were used in Example 2: pBK-lib2, plasmid DNA encoding a library of M. jannaschii tyrosyl t-RNA synthetase (TyrRS) variants randomized at residues Tyr32, Leu65, Phe108, Gln109, Asp 158 and Leu162 and containing a Kn^{r} marker; pREP(2)/YC, plasmid encoding mu tRNA_{CUA}^{Tyr} chloramphenicol acetyltransferase (CAT) gene with an amber TAG at Asp112 and GFP gene under control of the T7 promoter and its cognate amber mutant T7 RNA polymerase, and containing Tet^{r} marker; pLWJ17B3, plasmid expressing mu tRNA_{CUA}^{Tyr} under the control of the lpp promoter and rrnC terminator, and the barnase gene with three amber codons at Gln2, Asp44 and Gly65 under the control of the ara promoter, and containing Amp^{r} marker; pBAD/JYAMB-75TAG-Myo, pBAD/JYAMB-4TAG-Myo, plasmids containing the mutant sperm whale myoglobin gene (presence of amber mutation corresponding to either amino acid position 75 or 4 in the protein) on an arabinose promoter and rrnB terminator, mu tRNA^{tyr}_{CUA} on a lpp promoter and rrnC terminator and a tetracycline resistance marker; pBAD/JYAMB-125TAG-CAP, pBAD/JYAMB-70TAG-CAP, similar plasmids as described previously, except contain the E. coli catabolite activator protein (CAP) gene with amber mutation corresponding to amino acid 125 and 70; pBAD/JYAMB-601TAG-βGal, plasmid similar to the above pBAD/JYAMB constructs, but containing the β galactosidase gene lacZ having an amber mutation corresponding to amino acid Phe 601 of the protein; and, GeneHog®-Fis Cells (Invitrogen), F⁻ mcrA Δ(mrr-hsdRMS-mcrBC) 80lacZΔM15 ΔlacX74 recA1 endA1 araD139 Δ(ara-leu)7697 galU galK λrpsL(Str^{R}) nupG, fis::Tn7 (DHFR).

Genetic Selection of the Mutant Synthetase which Encodes for azobenzyl-Phe.

pBK-lib consisting of about 10⁹ TyrRS independent clones was constructed by an overlapping fragment PCR approach. Pfu Ultra from Stratagene (La Jolla, Calif.) was used for all PCRs using the manufacturer's protocol. E. coli DH10B harboring the pREP(2)/YC plasmid was used as the host strain for positive selection, into which the starting pBK-lib2 was transformed. Transformants were recovered in SOC for 1 hour, then washed twice in the cold with glycerol minimal media with leucine (GMML) before plating on GMML-agar plates supplemented with kanamycin, chloramphenicol and tetracycline at 50 ug/mL, 60 ug/mL and 12 ug/mL respectively. In addition, azobenzyl-Phe was present at 1 mM final concentration. In all, 6 GMML-agar plates with added azobenzyl-Phe and 1 control plate (with no azobenzyl-Phe) were used. The plates were incubated at 37° C. for 60 hours. Surviving cells were pooled from the 6 plates (with azobenzyl-Phe) and plasmid DNA was extracted. The pBK-lib2 DNA representative of the surviving clones was then resolved from the positive selection plasmid pREP(2)/YC by agarose gel electrophoresis. It was then transformed into electrocompetent negative selection strain harboring the pLWJ17B3 plasmid, recovered for 1 hour in 1 mL of LB and then plated on LB-agar plates (six in all) containing arabinose (0.2% wt/vol) and ampicillin (50 ug/mL). The plates were then incubated at 37° C. for 12 hours. Again, the resultant pBK-lib2 DNA of the surviving clones was recovered by the method described as above. This pBK-lib2 DNA was then carried through a subsequent round of positive selection (using 6 GMML-agar plates with azobenzyl-Phe and increasing chloramphenicol to 80 ug/mL) followed by a negative selection (6 LB-agar plates) and finally ending with a round of positive selection (4 GMML-agar plates; chloramphenicol at 100 and 120 ug/mL-2 plates each). At this stage, 96 individual synthetase clones were selected and each was suspended in 100 uL of GMML in a 96-well plate. A volume of 2 uL of suspension for each of the 96 wells was then replica-spotted on two sets of GMML plates. One set of GMML-agar plates was supplemented with tetracycline (12 ug/mL), kanamycin (50 ug/mL) and chloramphenicol at concentrations of 80, 100 and 120 ug/mL. Further, the unnatural amino acid azobenzyl-Phe was present at 1 mM. The other set of plates were identical in tetracycline and kanamycin but contained no azobenzyl-Phe. Additionally, chloramphenicol concentrations used were 0, 10, 20 and 40 ug/mL. After 48 hours of incubation at 37° C., 10 synthetase variants were selected for further analysis. Cells harboring these mutant synthetases showed a chloramphenicol-dependant growth to 120 ug/mL in presence of 1 mM azobenzyl-Phe and to 20 ug/mL in absence of the unnatural amino acid. Standard method of DNA sequence analysis of all the 10 mutant synthetase encoding plasmids revealed convergence to a single mutant synthetase sequence (Gly32, Glu65, Ala108, Glu109, Gly158, His162). This mutant synthetase (termed azobenzyl-PheRS) which specifically charges phenylalanine-4′-azobenzene (azobenzyl-Phe) was selected from the pBK-lib2 as described above.

Expression of Mutant Myoglobin and Catabolite Activator Protein (CAP)

Plasmid pBAD/JYAMB-75TAG-Myo was cotransformed with pBK vector expressing azobenzyl-PheRS (pBK-AzoPheRS) into GeneHog®-Fis E. coli cells. Cells were amplified in LB media (5 mL) supplemented with kanamycin (40 ug/mL) and tetracycline (24 ug/1 mL) followed by washing (twice) with PBS. The starter culture (8 uL) was used to inoculate a 150 mL of liquid GMML supplemented with appropriate antibiotics and azobenzyl-Phe (1 mM). Cells were then grown at 37° C. to an OD₆₀₀ of 0.5 when protein expression was induced by the addition of 0.02% arabinose. After another 8 hours of growth, these cells were harvested by centrifugation. Then the mutant protein (myoglobin with azobenzyl-Phe at position 75) was purified by virtue of a C-terminal hexahistidine tag using Ni-NTA affinity chromatography under non-denaturing conditions. Samples were desalted, eluted in water and subjected to MALDI-TOF mass spectrometric analysis. Expected M_{avg} 18535.172; Observed M_{avg} 18534. ESI analysis gave Observed M_{avg} 18535. A control expression was carried out exactly as above and simultaneously in the absence of azobenzyl-Phe.

Mutant sperm whale myoglobin protein having azobenzyl-Phe at position 4 was similarly expressed as above from the plasmid pBAD/JYAMB-4TAG-Myo. The protein sequence of this mutant myoglobin was then analyzed for the first 6 amino acids from the N terminus on an Applied Biosystems PROCISE 494HT Protein Sequenator at UCSD, Division of Biological Sciences Protein Sequencing Facility. PTH-azobenzyl-Phe (i.e., phenylthiohydantoin-phenylalanine-4′-azobenzene) synthesized above was used as the standard. The HPLC profile of the PTH-amino acids was analyzed at 269 nm. The gradient was run from solvent A (3.5% tetrahydrofuran in water, containing 20 ml of ABI's Premix buffer concentrate, 900 microliter of 1% acetone in water, 75 microliter of TFA, 100 microliter of 1M KH₂PO₄ per 960 ml of the solvent A to solvent B (12% isopropanol in acetonitrile).

Catabolite activator protein (CAP) of E. coli having a Leu 125 azobenzyl-Phe mutation was expressed from pBAD/JYAMB-125TAG-CAP in the same way as the mutant myoglobin proteins, except rich media (2YT) expression in presence of 0.002% arabinose induction was carried out. Yield of this mutant CAP was 1.5-1.75 mg/liter of cell culture. UV-visible absorption spectra of both the dark-adapted (predominantly trans) and the photoirradiated samples of mutant CAP were then taken on a double beam UV-visible spectrophotometer (Uvikon 933 from Kontron Instruments, Bletchley, UK). A high pressure mercury lamp (500 W; from Spectra Physics, Mt. View, Calif.) equipped with a mounted long pass optical filter (320 nm; from Spectra Physics) and interchangeable narrow bandpass interference filters (334 nm and 420 nm; band width±5 nm; from Edmund Optics, Barrington, N.J.) was used for photoirradiation of the azobenzyl-Phe incorporated mutant CAP at 25° C.

Photomodulation of Protein—DNA Interactions In Vitro.

Isolation of E. coli lactose promoter segment containing the primary CAP binding site. See Hudson, et al., J. Bacteriol., 1991, 173:59-66. Plasmid pUC19 propagated in E. coli DH10B, was purified using the QIAfilter Plasmid Mega Kit (Qiagen, Valencia, Calif.). A 214 bp lactose promoter fragment containing both the primary and secondary CAP binding sites was isolated upon Hinf1 restriction digest of pUC19 and preparative polyacrylamide gel electrophoresis. Further endonuclease digestion by HpaII of the 214 bp fragment, followed by separation on a 10% polyacrylamide-TBE gel (40 cm×20 cm−length×width) led to the isolation of the 118 bp lactose promoter fragment containing only the CAP primary binding site. To remove the 5′ phosphate, the 118 bp fragment was then treated with calf intestinal alkaline phosphatase (CIP) and purified using the Qiagen Nucleotide Removal Kit. The 5′ termini of the 118 bp fragment were labeled with ³²P according to the method of Maxam and Gilbert. See Maxam, et al., Proc. Natl. Acad. Sci. USA, 1977, 74:560-4.

Mutant CAP protein expression. A photoswitchable CAP mutant having the Ile 70 azobenzyl-Phe mutation was expressed similarly from plasmid pBAD/JYAMB-70TAG-CAP (that had been cotransformed into GeneHog®-Fis E. coli cells containing plasmid pBK-azobenzyl-PheRS) in the same way as the mutant myoglobin proteins, except rich media (2YT) expression in presence of 0.002% arabinose induction was carried out. The CAP wild-type protein was also isolated using the pBAD/JYAMB-CAPWT plasmid. The purified proteins were exchanged into 50 mM sodium phosphate buffer (plus 300 mM NaCl, pH 8) and the protein concentrations determined spectrophotometrically (ε₂₈₀=3.5×10⁴ M⁻¹ cm⁻¹/CAP dimer). See Anderson, et al., J. Biol. Chem., 1971, 246:5929-37.

Formation of CAP-DNA complexes and Analysis. In general, protein-DNA binding reactions were initiated by the addition of 160 nM of mutant or wild-type CAP to 33 nM of 5′ ³²P-labeled lac promoter fragment containing the primary CAP binding site. The incubation was carried out at 25° C. for 1 hour. The binding reaction buffer was made of 10 mM Tris (pH 7.5), 50 mM NaCl, 500 uM EDTA, 500 uM DTT, 1 mM MgCl₂, 4% glycerol and 20 uM cAMP. After incubation, the binding reaction was mixed with loading buffer and then loaded directly into the wells of a 6% TBE-PAGE gel that had been pre-run (30 minutes, 150 V) with the running buffer (0.089M Tris Base, 0.089M Boric Acid, and 0.002M EDTA, pH 8.3) which additionally contained 20 uM cAMP. Gel electrophoresis of the binding reaction samples was carried out at 150 V for 45 minutes. On completion, the gel was blotted free of excess buffer, wrapped in a plastic wrap and then exposed to a storage phosphor screen for 1 hour. Following this, the screen was imaged on a Storm® Phosphorimager System (Molecular Dynamics, Piscataway, N.J.).

For gel-shift assays with the photoswitchable protein, the samples of the mutant CAP (Ile 70 azobenzyl-Phe) in 50 mM phosphate buffer, pH 8 were photoirradiated at 334 nm at 0° C. for 40 minutes prior to binding with the lactose promoter fragment.

For determination of the binding constant (see Fried, et al., J. Mol. Biol., 1984, 172:241-62) of CAP (wild-type or mutant CAP70azobenzyl-Phe) to its primary binding site on the lac promoter, a typical three-fold serial dilution of the protein was made from a starting concentration of 1.6×10⁻⁶ M. The lac promoter fragment and cAMP were maintained at a constant 33 nM and 20 uM respectively. For each dilution step, the binding reaction of CAP to the DNA fragment was allowed to reach equilibration and analyzed by gel electrophoresis as described previously. From the relative intensities of the radioactive bands in the electromobility shift assay, the fraction of bound DNA (PC/P₀) and free DNA (P/P₀) was determined, where P₀ represents the total DNA, PC represents the DNA bound to CAP, P represents the free DNA upon DNA-CAP equilibration. Further, C₀ and C stand for total protein and unbound protein respectively. A linear regression analysis of the plot of log([PC]/[P]) versus log [C], provides the binding constant of the CAP-DNA interaction (K_{b}) from the interpolation on the x-axis.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A translation system comprising;
an orthogonal tRNA (O-tRNA) or modified variant thereof; and,
an orthogonal aminoacyl tRNA synthetase (O-RS) that preferentially charges the orthogonal tRNA, or modified variant thereof, with one or more amino acid, which amino acid is selected from the group consisting of: α-aminocaprylic acid, o-nitrobenzyl cysteine, and azobenzyl-Phe, or an O-RS or modified variant thereof comprising a sequence of SEQ ID NO: 9-12, that preferentially charges the O-tRNA or modified variant thereof with o-methyl tyrosine.

2. The translation system of claim 1, wherein the translation system comprises a cell.

3. The translation system of claim 2, wherein the cell is a yeast cell or wherein the cell is a eubacterial cell.

4. The translation system of claim 1, wherein the amino acid is an unnatural amino acid.

5. The translation system of claim 1, wherein the O-tRNA is a modified leucyl-O-tRNA.

6. The translation system of claim 1, wherein the O-tRNA is a modified tyrosyl-O-tRNA.

7. The translation system of claim 1, wherein the O-tRNA or modified variant thereof, the O-RS, or both the O-tRNA and the modified variant thereof, are derived from E. coli.

8. The translation system of claim 1, wherein the O-tRNA or modified variant thereof, the O-RS, or both the O-tRNA and the modified variant thereof, are derived from M. jannaschii.

9. The translation system of claim 1, wherein the O-RS is derived from the wild-type E. coli-tRNA synthetase having the amino acid sequence of SEQ ID NO: 3.

10. The translation system of claim 1, wherein the O-RS is derived from the wild-type M. jannaschii tRNA synthetase having the amino acid sequence of SEQ ID NO: 4.

11. The translation system of claim 1, wherein the O-RS is derived from the wild-type E. coli tRNA synthetase having the amino acid sequence of SEQ ID NO: 3, wherein the O-RS has an amino acid sequence comprising:
(a) Ala, Val, His, Leu, Met, Phe, Gly, or Trp at amino acid position 40;
(b) Ala, Met, Pro, Tyr, Glu, Trp, Ser, or Thr at amino acid position 41;
(c) Pro, Leu, Ala, Arg, Ile, or Trp at amino acid position 499;
(d) Val, Leu, Met, Ala, Phe, Cys, or Thr at amino acid position 527; and
(e) Gly at amino acid position 537.

12. The translation system of claim 1, wherein the O-RS is derived from the wild-type M. jannaschii tRNA synthetase having the amino acid sequence of SEQ ID NO: 4, wherein the O-RS has an amino acid sequence comprising:
(a) Gly at amino acid position 32;
(b) Glu at amino acid position 65;
(c) Ala at amino acid position 108;
(d) Glu at amino acid position 109;
(e) Gly at amino acid position 158; and,
(f) His at amino acid position 162.

13. The translation system of claim 1, wherein the O-RS comprises an amino acid sequence selected from SEQ ID NO:5-17, and conservative variants thereof

14. The translation system of claim 1, wherein the system comprises a polynucleotide encoding the O-RS, wherein the O-RS comprises an amino acid sequence selected from SEQ ID NO:5-17, and conservative variants thereof.

15. The translation system of claim 14, wherein the polynucleotide is selected from the nucleotide sequences of SEQ ID NO:20-32.

16. The translation system of claim 1, wherein the O-tRNA comprises, or is encoded by, a polynucleotide sequence set forth in SEQ ID NO: 1-2.

17. The translation system of claim 1, comprising a nucleic acid comprising a first O-RS and at least one selector codon, wherein said selector codon is recognized by a first O-tRNA.

18. The translation system of claim 17, comprising a second O-RS and a second O-tRNA, wherein the second O-RS preferentially aminoacylates the second O-tRNA with a second amino acid that is different from the first amino acid, and wherein the second O-tRNA recognizes a selector codon that is different from the selector codon recognized by the first O-tRNA.

19. The translation system of claim 1, wherein the O-tRNA or modified variant thereof comprises a recognition sequence for an amber codon.

20. The translation system of claim 1, comprising a target nucleic acid comprising an amber codon.

21. The translation system of claim 20, comprising a protein encoded by the target nucleic acid.

22. The translation system of claim 21, wherein the protein comprises a photoregulated amino acid.

23. The translation system of claim 22, wherein the protein comprises azobenzyl-Phe or o-nitrobenzyl cysteine.

24. A protein produced by the translation system of claim 1.

25. The protein of claim 24, wherein the protein comprises an unnatural amino acid.

26. The protein of claim 25, wherein the unnatural amino acid is α-aminocaprylic acid, O-methyl tyrosine, o-nitrobenzyl cysteine, or azobenzyl-Phe.

27. A composition comprising the protein of claim 24.

28. A composition comprising an orthogonal aminoacyl-tRNA synthetase (O-RS), wherein the O-RS preferentially aminoacylates an O-tRNA with α-aminocaprylic acid, o-nitrobenzyl cysteine, or azobenzyl-Phe, or wherein the O-RS comprises the sequence of SEQ ID NO: 9-12, and preferentially aminoacylates an O-tRNA with o-methyl tyrosine.

29. The composition of claim 28, wherein the O-tRNA is a leucyl-O-tRNA.

30. The composition of claim 28, wherein the O-tRNA is a tyrosyl-O-tRNA.

31. The composition of claim 28, wherein the O-RS comprises an amino acid sequence of SEQ ID NO: 5-17 or a conservative variation thereof.

32. The composition of claim 28, wherein the O-RS preferentially aminoacylates the O-tRNA with an efficiency of at least 50% of the efficiency of any one of SEQ ID NO: 5-8 and 13-17.

33. The composition of claim 28, wherein the O-RS is derived from E. coli.

34. The composition of claim 28, wherein the O-RS is derived from M. jannaschii.

35. The composition of claim 28, wherein the O-tRNA recognizes an amber selector codon.

36. The composition of claim 27, comprising a cell, wherein the O-RS is encoded by one or more nucleic acids in the cell, wherein the nucleic acids are chosen from SEQ ID NO: 20-32 or a conservative variation thereof.

37. The composition of claim 36, wherein the cell is a yeast cell.

38. The composition of claim 27, comprising a translation system.

39. The composition of claim 27, comprising a cell, wherein the O-RS is encoded by one or more nucleic acids in the cell, the cell further comprising:
an orthogonal tRNA (O-tRNA); and,
one or more of α-aminocaprylic acid, O-methyl tyrosine, o-nitrobenzyl cysteine, or azobenzyl-Phe;
wherein the O-tRNA recognizes a selector codon, and the O-RS preferentially aminoacylates the O-tRNA with one of α-aminocaprylic acid, O-methyl tyrosine, o-nitrobenzyl cysteine, or azobenzyl-Phe.

40. The composition of claim 39, wherein the cell comprises a target nucleic acid that encodes a polypeptide of interest, wherein the target nucleic acid comprises a selector codon that is recognized by the O-tRNA.

41. A nucleic acid that encodes any one of SEQ ID NO: 5-17, or a conservative variation thereof.

42. The nucleic acid of claim 41, wherein the nucleic acid is chosen from SEQ ID NO: 20-32.

43. A protein comprising one or more of α-aminocaprylic acid, o-nitrobenzyl cysteine, or azobenzyl-Phe.

44. A composition comprising a protein of claim 43.

45. A method for selecting an active orthogonal aminoacyl-tRNA synthetase (O-RS) that charges an α-aminocaprylic acid, o-nitrobenzyl cysteine, or azobenzyl-Phe on an orthogonal tRNA (O-tRNA), the method comprising:
subjecting a population of cells to selection, wherein the cells collectively comprise:
the O-tRNA, wherein the O-tRNA is orthogonal to members of the population of cells that comprise the O-tRNA;
a plurality of O-RS that comprises one or more active O-RS members that load the O-tRNA with an α-aminocaprylic acid, o-nitrobenzyl cysteine, or azobenzyl-Phe in one or more cells of the population;
a polynucleotide that encodes a selectable marker, wherein the polynucleotide comprises at least one selector codon that is recognized by the O-tRNA; and,
α-aminocaprylic acid, o-nitrobenzyl cysteine, or azobenzyl-Phe;
wherein a target cell in the population that comprises the active O-RS is identified by an enhanced suppression efficiency of the selectable marker as compared to a suppression efficiency of a control cell lacking the plurality of RS but comprising the O-tRNA; and, selecting the target cell, thereby selecting the active O-RS.

46. The method of claim 45, wherein the cells are additionally selected to eliminate cells that comprise a non-target O-RS that charges the O-tRNA with an amino acid other than α-aminocaprylic acid, o-nitrobenzyl cysteine, or azobenzyl-Phe.

47. The method of claim 45, wherein the selection comprises a positive selection and the selectable marker comprises a positive selection marker.

48. The method of claim 45, wherein the O-tRNA is leucyl-O-tRNA.

49. The method of claim 45, wherein the O-tRNA is tyrosyl-O-tRNA.

50. An orthogonal aminoacyl-tRNA synthetase identified by the method of claim 45.

51. A method of producing a protein in a cell, which protein comprises one or more α-aminocaprylic acid, o-nitrobenzyl cysteine, azobenzyl-Phe, photoregulated serine, photoregulated serine analogue, fluorophore, spin labeled amino acid, or an amino acid comprising a dansyl side chain at one or more specified position, the method comprising:
growing the cell in an appropriate medium, which cell comprises a nucleic acid that comprises at least one selector codon and that encodes a protein; and,
providing α-aminocaprylic acid, o-nitrobenzyl cysteine, azobenzyl-Phe, photoregulated serine, a photoregulated serine analogue, a fluorophore, a spin labeled amino acid, or an amino acid comprising a dansyl side chain;
which cell further comprises:
an orthogonal tRNA (O-tRNA) that recognizes the selector codon; and,
an orthogonal aminoacyl-tRNA synthetase (O-RS) that preferentially aminoacylates the O-tRNA with the α-aminocaprylic acid, o-nitrobenzyl cysteine, azobenzyl-Phe, photoregulated serine, a photoregulated serine analogue, a fluorophore, a spin labeled amino acid, or an amino acid comprising a dansyl side chain; and,
incorporating the α-aminocaprylic acid, o-nitrobenzyl cysteine, azobenzyl-Phe, photoregulated serine, a photoregulated serine analogue, a fluorophore, a spin labeled amino acid, or an amino acid comprising a dansyl side chain into the specified position in response to the selector codon, thereby producing the protein.

52. The method of claim 51, wherein the O-RS comprises an amino acid sequence corresponding to SEQ ID NO: 5-17, or a conservative variation thereof.

53. A library of polynucleotide members useful for the identification of an orthogonal aminoacyl-tRNA synthetase (O-RS) that functions in a host cell, wherein said polynucleotide members encode variants of an amino acid sequence selected from:
(i) an amino acid sequence set forth in SEQ ID NO: 4, said polynucleotide members comprising randomized nucleotide positions in codons encoding Tyr³², Leu⁶⁵, Phe¹⁰⁸, Gln¹⁰⁹, Asp¹⁵⁸ and Leu¹⁶² in SEQ ID NO: 4; or
(ii) an amino acid sequence of an Archaea aminoacyl-tRNA synthetase other than the amino acid sequence set forth in SEQ ID NO: 4, said polynucleotide members comprising randomized nucleotide positions in codons whose corresponding amino acid positions spatially correspond to Tyr³², Leu⁶⁵, Phe¹⁰⁸, Gln¹⁰⁹, Asp¹⁵⁸ and Leu¹⁶² in SEQ ID NO: 4.

54. The library of claim 53, wherein said polynucleotide members comprise an expression vector.

55. The library of claim 53, wherein said O-RS preferentially aminoacylates an orthogonal tRNA (O-tRNA) with an unnatural amino acid.

56. The library of claim 53, wherein said O-RS comprises one or more conservative amino acid substitutions at positions other than (i) positions 32, 65, 108, 109, 158 and 162 in SEQ ID NO: 4; or (ii) positions that spatially correspond to Tyr³², Leu⁶⁵, Phe¹⁰⁸, Gln¹⁰⁹, Asp¹⁵⁸ and Leu¹⁶² in SEQ ID NO: 4.

57. The library of claim 53, wherein said host cell is an E. coli cell.

58. A plurality of cells comprising a plurality of library polynucleotide members of claim 53.

59. The library of claim 53, wherein said Archaea aminoacyl-tRNA synthetase is a Methanococcus jannaschii aminoacyl-tRNA synthetase.

60. The library of claim 59, wherein said Methanococcus jannaschii aminoacyl-tRNA synthetase is a Methanococcus jannaschii tyrosyl-tRNA synthetase.

61. A method for identifying a desired orthogonal aminoacyl-tRNA synthetase (O-RS), the method comprising:
a) providing
(i) a library of polynucleotide members encoding variants of an amino acid sequence set forth in SEQ ID NO: 4, said polynucleotide members comprising randomized nucleotide positions in codons encoding Tyr32, Leu65, Phe108, Gln109, Asp158 and Leu162 in SEQ ID NO: 4; and
(ii) a host cell; and
b) detecting a polynucleotide member from said library that encodes a polypeptide that preferentially aminoacylates an orthogonal tRNA (O-tRNA) with an unnatural amino acid in said host cell, thereby identifying a desired O-RS.

62. The method of claim 61, wherein said detecting step comprises a positive selection made by expressing a chloramphenicol acetyltransferase protein and detecting cell survival in the presence of chloramphenicol.

63. The method of claim 62, wherein said detecting step comprises a negative selection made by expressing a barnase protein.

64. A library of polynucleotide members useful for the identification of an orthogonal aminoacyl-tRNA synthetase (O-RS) that functions in a host cell, wherein said polynucleotide members encode variants of an amino acid sequence selected from:
(i) an amino acid sequence set forth in SEQ ID NO: 3, said polynucleotide members comprising randomized nucleotide positions in codons encoding Met⁴⁰, Leu⁴¹, Tyr⁴⁹⁹, Tyr⁵²⁷, and His137 in SEQ ID NO: 3; or
(ii) an amino acid sequence of an Eubacterial aminoacyl-tRNA synthetase other than the amino acid sequence set forth in SEQ ID NO: 3, said polynucleotide members comprising randomized nucleotide positions in codons whose corresponding amino acid positions spatially correspond to Met⁴⁰, Leu⁴¹, Tyr⁴⁹⁹, Tyr⁵²⁷, and His⁵³⁷ in SEQ ID NO: 3.

65. The library of claim 64, wherein said polynucleotide members comprise an expression vector.

66. The library of claim 64, wherein said O-RS preferentially aminoacylates an orthogonal tRNA (O-tRNA) with an unnatural amino acid.

67. The library of claim 64, wherein said O-RS comprises one or more conservative amino acid substitutions at positions other than (i) positions 40, 41, 499, 527, and 537 in SEQ ID NO: 3; or (ii) positions that spatially correspond to Met⁴⁰, Leu⁴¹, Tyr⁴⁹⁹, Tyr⁵²⁷, and His⁵³⁷ in SEQ ID NO: 3.

68. The library of claim 64, wherein said host cell is an S. cerevisiae cell.

69. A plurality of cells comprising a plurality of library polynucleotide members of claim 64.

70. The library of claim 64, wherein said Eubacterial aminoacyl-tRNA synthetase is an Escherichia coli aminoacyl-tRNA synthetase.

71. The library of claim 70, wherein said Escherichia coli aminoacyl-tRNA synthetase is a Escherichia coli leucyl-tRNA synthetase.

72. A method for identifying a desired orthogonal aminoacyl-tRNA synthetase (O-RS), the method comprising:
a) providing
(i) a library of polynucleotide members encoding variants of an amino acid sequence set forth in SEQ ID NO: 3, said polynucleotide members comprising randomized nucleotide positions in codons encoding Met⁴⁰, Leu⁴¹, Tyr⁴⁹⁹, Tyr¹²¹, and His⁵³⁷ in SEQ ID NO: 3; and
(ii) a host cell; and
b) detecting a polynucleotide member from said library that encodes a polypeptide that preferentially aminoacylates an orthogonal tRNA (O-tRNA) with an unnatural amino acid in said host cell, thereby identifying a desired O-RS.

73. The method of claim 72, wherein said detecting step comprises a positive selection made by expressing a gal4 protein and detecting cell survival in the absence of uracil or in the absence of histidine, but in the presence of aminotriazole.

74. The method of claim 72, wherein said detecting step comprises a negative selection made by expressing a ura3 protein in the presence of fluorootic acid.

75. A method of modulating an activity of a protein, the method comprising:
a) incorporating an azobenzyl-Phe or o-nitrobenzyl cysteine into the protein via an O-RS and O-tRNA pair that are specific for azobenzyl-Phe or o-nitrobenzyl cysteine;
b) exposing the protein to a wavelength of light energy that photoregulates the azobenzyl-Phe or o-nitrobenzyl cysteine, thereby modulating the activity of the protein comprising the azobenzyl-Phe or o-nitrobenzyl cysteine.

76. A system for modulating an activity of a protein, the system comprising:
a) a protein comprising azobenzyl-Phe or o-nitrobenzyl cysteine;
b) a light source which photoregulates the azobenzyl-Phe or o-nitrobenzyl cysteine of the protein, thereby modulating the activity of the protein.
